(19) United States
(12) Patent Application Publication
(10) Pub. No.: US 2005/0266552 A1
(43) Pub. Date: Dec. 1, 2005

(54) Reagents and methods for identification of RNAi pathway genes and chemical modulators of RNAi

(76) Inventors: John G. Doench, Somerville, MA (US); Derek M. Dykxhoorn, Watertown, MA (US); Carl D. Novina, Newton, MA (US); Phillip A. Sharp, Newton, MA (US)

Correspondence Address: CHOATE, HALL & STEWART LLP, TWO INTERNATIONAL PLACE, BOSTON, MA, 02110, US

(21) Appl. No.: 11/005,127

(22) Filed: Dec. 5, 2004

Related U.S. Application Data
(60) Provisional application No. 60/527,872, filed on Dec. 5, 2003.
(60) Provisional application No. 60/529,644, filed on Dec. 15, 2003.

(51) Int. Cl. C12P 21/06; C12N 5/06; C12N 5/08; C12N 15/85

(52) U.S. Cl. 435/358; 435/455; 435/367

(57) ABSTRACT

The present invention provides reagents such as cells, cell lines, and vectors, that can be used to identify mammalian genes whose expression products (RNA or protein) play a role in RNA interference (RNAi) and/or to identify chemical modulators of RNAi, or for other purposes. The invention further provides a variety of methods for identifying such genes or modulators. In particular, the invention provides a mammalian cell comprising a nucleic acid that encodes a selectable marker and one or more nucleic acid templates for transcription of an RNAi-inducing agent integrated into the genome of the cell, wherein the RNAi-inducing agent reduces expression of the marker and is not naturally found in the cell. Additional cells and cell lines comprising nucleic acids that encode one or more additional markers are also provided. According to certain of the inventive methods cells such as these are mutagenized, transfected or infected with a library of genetic suppressor elements, or contacted with a test compound. Cells in which RNAi is inhibited or activated are identified using an appropriate selective condition or screening method. The identity of the mutated or inhibited gene or the identity of the compound is then determined.

### CROSS-REFERENCE TO RELATED APPLICATION

This application claims priority to U.S. Provisional Patent Application 60/527,872, filed Dec. 5, 2003 and U.S. Provisional Patent Application 60/529,644, filed Dec. 15, 2003. The contents of each of these applications are incorporated herein by reference.

### GOVERNMENT SUPPORT

The United States Government has provided grant support utilized in the development of the present invention. In particular, National Institutes of Health grant numbers R01-AI32486 and F32 AI10523-02 and National Cancer Institute grant number P01-CA42063 have supported development of this invention. The United States Government may have certain rights in the invention.

### BACKGROUND OF THE INVENTION

In 1998, Fire and Mello described a new technology in which injection of double-stranded RNA (dsRNA) induces potent and specific gene silencing in Caenorhabditis elegans through a process referred to as RNA interference (RNAi). Recapitulation of the RNAi reaction in Drosophila melanogaster embryo extracts demonstrated that long dsRNA substrates could be cleaved into short dsRNA species approximately 21-23 nucleotides in length. It was further shown that addition of chemically synthesized ˜21-22 nucleotide RNA duplexes to these extracts resulted in sequence specific degradation of messenger RNA (mRNA) containing a region homologous to the antisense strand of the duplex. Such small RNAs, capable of mediating sequence specific gene silencing, were named short interfering RNAs (siRNAs). Subsequent studies revealed that RNAi operates in a wide variety of species, including in mammalian cells in tissue culture and in both embryonic and non-embryonic mammalian organisms.

Since its initial discovery RNAi has rapidly been adopted as a strategy for gene silencing both in vitro and in vivo in a number of tissue culture systems and organisms. Applications include studies designed to elucidate gene function by “knockdown” of expression of endogenous genes. In addition, a number of potential therapeutic applications have been demonstrated. For example administration of siRNAs targeted to certain viral or cellular genes has been shown to inhibit replication of viruses in mammalian cells, and various oncogenes have also been targeted.

Biochemical characterization of siRNAs generated from longer dsRNA molecules led to the identification of a conserved family of RNase III-like enzymes named Dicer, members of which cleave longer dsRNA molecules into siRNAs. SiRNAs generated intracellularly or introduced into cells are incorporated into a multiprotein RNA-induced silencing complex (RISC), which ultimately results in cleavage of the target mRNA.

Although much has been learned about the mechanisms that are involved in RNAi, a great deal remains poorly understood. For example, many of the molecular components that mediate RNAi remain unidentified, particularly those that operate in mammalian cells. In order to fully exploit the potential of RNAi there exists a need in the art for reagents and methods that can be used to identify such components and/or that can be used to identify reaction conditions that may influence the efficacy of RNAi. In addition, there is a need for molecules that can regulate, control, or modulate, e.g., enhance or inhibit RNAi pathways and for tools that would allow identification of such molecules. There is also a need in the art for improved methods to isolate and/or enrich for functional small RNAs, e.g., small RNAs that are involved in physiologically relevant processes such as transcriptional and/or translational control of gene expression.

### SUMMARY OF THE INVENTION

The present invention addresses the foregoing needs, among others. The invention provides cells, cell lines, vectors, and other reagents that are useful for identification of inhibitors and/or activators of RNAi in eukaryotic cells, preferably mammalian cells, and for various other purposes. The invention further provides reagents and systems that are useful for identification of genes whose expression products (RNA or protein) are involved in mediating RNAi, i.e., genes whose expression products function in one or more RNAi pathways. In addition, the invention provides methods for identifying inhibitors and/or activators of RNAi, e.g., chemical modulators of RNAi such as small molecules. Various methods and reagents described herein may be used to selectively identify genes and/or chemical modulators of siRNA RNAi pathways and/or microRNA (miRNA) translational repression pathways.

In one aspect, the invention provides a mammalian cell comprising: (i) a nucleic acid that encodes a selectable marker; and (ii) one or more nucleic acid templates for transcription of an RNAi-inducing agent integrated into the genome of the cell, wherein the RNAi-inducing agent reduces expression of the marker and is not naturally found in the cell. In certain embodiments of the invention the cell further comprises (i) a nucleic acid that encodes a detectable marker; and (ii) one or more templates for transcription of an RNAi-inducing agent that reduces expression of the detectable marker. In certain embodiments of the invention either of the foregoing cells may further comprise (i) a nucleic acid that encodes a second selectable marker; and (ii) one or more templates for transcription of an RNAi-inducing agent that reduces expression of the second selectable marker. Certain preferred selectable markers include hypoxanthine-guanine phosphoribosyl transferase (HPRT), thymidine kinase (TK), and multidrug resistance (MDR) family proteins. Certain preferred detectable markers include markers that produce a fluorescent, luminescent, or colorimetric signal, e.g., green fluorescent protein (GFP) or an enhanced and/or destabilized version of GFP. In general, the nucleic acids that provide templates for transcription of an RNAi-inducing agent are operably linked to a promoter, e.g., a promoter recognized by RNA polymerase I or RNA polymerase III, such as a U6, H1, or tRNA promoter. The cells synthesize an RNAi-inducing agent that silences a transcript that encodes the selectable or detectable marker. By detecting derepression of silencing, e.g., after mutagenizing or otherwise manipulating the cells, or after contacting them with a test compound, genes and/or compounds that modulate RNAi may be identified.

In another aspect, the invention provides a cell comprising: (i) a nucleic acid that encodes a detectable marker, wherein the detectable marker has a half-life of approximately 2 hours or less; and (ii) a template for transcription of an RNAi-inducing agent that reduces expression of the detectable marker integrated into the genome of the cell, wherein the RNAi-inducing agent reduces expression of the marker and is not naturally found in the cell.

The invention further provides cell lines each comprising a plurality of cells such as those described above. In addition, the invention provides a collection of cell lines wherein cells of each cell line comprise (i) a nucleic acid that encodes a marker, wherein the nucleic acid in cells of each cell line encodes the same marker; and (ii) a template for transcription of an RNAi-inducing agent that reduces expression of the marker, wherein the RNAi-inducing agent reduces expression of the marker to different extents in each of the cell lines.

In another aspect, the invention provides a nucleic acid comprising: (i) a template for transcription of a first RNAi-inducing agent targeted to an mRNA that encodes a first marker, wherein the template is operably linked to a promoter active in a mammalian cell; and (ii) a template for transcription of a second RNAi-inducing agent targeted to an mRNA that encodes a second marker, wherein the template is operably linked to a promoter active in a mammalian cell. The invention further provides a nucleic acid comprising (i) a template for transcription of a first RNAi-inducing agent targeted to a selectable or detectable marker and operably linked to a first promoter; and (ii) a second promoter and a site for insertion of a template for transcription of an RNAi-inducing agent located downstream of the promoter, so that the template will be operably linked to the promoter once inserted. The invention further provides vectors, cells, and cell lines containing any of the foregoing nucleic acids.

In another aspect, the invention provides a method of identifying a cell in which a gene of interest is silenced by RNAi comprising steps of: (i) introducing a nucleic acid comprising (a) a template for transcription of a first RNAi-inducing agent targeted to an mRNA that encodes a first marker, wherein the template is operably linked to a promoter active in a mammalian cell; and (b) a template for transcription of a second RNAi-inducing agent targeted to an mRNA that encodes a second marker, wherein the template is operably linked to a promoter active in a mammalian cell, into a population of mammalian cells, wherein the nucleic acid further comprises a template for transcription of an RNAi-inducing agent targeted to the gene of interest; and (ii) identifying a cell in which RNAi is active by selecting or detecting cells that do not express the selectable or detectable marker, thereby identifying a cell in which the gene of interest is silenced by RNAi.

In additional aspects, the invention provides various methods for identifying genes that are involved in RNAi. For example, the invention provides a method of identifying a gene involved in an RNAi pathway comprising steps of: (a) providing a population of mammalian cells members of which comprise a nucleic acid that encodes a detectable or selectable marker and further comprise one or more templates for transcription of an RNAi-inducing agent that reduces expression of the detectable or selectable marker; (b) mutagenizing the population of cells; and (c) identifying cells that display decreased or increased expression of the detectable or selectable marker relative to the starting population, thereby identifying cells that have a mutation in a gene involved in an RNAi pathway. Another method for identifying cells containing a genetic element that inhibits or activates an RNAi pathway comprises steps of: (a) providing a first population of mammalian cells members of which comprise a nucleic acid that encodes a first detectable or selectable marker and express an RNAi-inducing agent that reduces expression of the detectable or selectable marker; (b) introducing a library into the population of cells, wherein the library comprises a plurality of genetic elements; and (c) identifying cells that display increased or decreased expression of the detectable or selectable marker relative to the starting population, thereby identifying cells that contain a genetic element that inhibits or activates an RNAi pathway, respectively.

In another aspect, the invention provides a method for identifying a compound that inhibits or activates RNA interference comprising steps of: (a) providing a population of mammalian cells members of which comprise a nucleic acid that encodes a detectable or selectable marker and express an RNAi-inducing agent that reduces expression of the detectable or selectable marker by RNA interference; (b) contacting the cells with a compound; and (c) identifying the compound as an inhibitor of RNAi if cells exhibit enhanced expression of the detectable or selectable marker after being contacted with the compound relative to cells not contacted with the compound or identifying the compound as an activator of RNAi if cells exhibit reduced expression of the detectable or selectable marker after being contacted with the compound relative to cells not contacted with the compound. According to certain embodiments of the invention a compound library, e.g., a library of small molecules, is screened to identify active compounds. The invention further provides compounds identified according to the inventive methods, methods for using the compounds, and pharmaceutical compositions including them.

The invention also provides a variety of kits containing one or more of the cell lines, nucleic acids, vectors, and/or compounds of the invention in addition to other components.

This application refers to various patents, journal articles, and other publications, all of which are incorporated herein by reference. In addition, the following standard reference works are incorporated herein by reference: Current Protocols in Molecular Biology, Current Protocols in Immunology, Current Protocols in Protein Science, and Current Protocols in Cell Biology, John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001. In the event of a conflict between the instant specification and an incorporated reference, the specification shall control.

### BRIEF DESCRIPTION OF THE DRAWING

FIG. 1A shows an example of an siRNA molecule. (From Dykxhoorn, D., et al., Molecular Cell Biology, 4:457-467, 2003).

FIG. 1B shows a schematic diagram of the short interfering (si)RNA pathway, one of the RNA interference pathways of interest herein.

FIG. 1C shows a schematic diagram of the micro(mi) RNA pathway, one of the RNA interference pathways of interest herein. (From Dykxhhoorn, D., supra).

FIG. 2 shows schematic diagrams of various RNAi-inducing agents, i.e., miRNA precursors, shRNAs, and siRNAs.

FIG. 3 shows a variety of methods for use in vitro to generate short RNAs that mediate RNAi.

FIG. 4 is a simplified schematic diagram of nucleotide synthesis pathways in mammalian cells highlighting steps that are blocked in the presence of aminopterin and showing key enzymes (DHFR and TK) of the salvage pathways that allow cells to circumvent the blocks in de novo synthesis.

FIG. 5 shows a portion of the de novo pathway of purine synthesis in mammalian cells illustrating the requirement for DHFR and the fact that aminopterin inhibits DHFR.

FIG. 6 shows a portion of the purine salvage pathway in mammalian cells illustrating the conversion of hypoxanthine to IMP and of guanine to GMP by HPRT. The figure also shows that the guanine analog 8-AZ inhibits HPRT, which leads to cell death in the presence of an inhibitor of DHFR.

FIG. 7 shows a DNA vector containing a U6 promoter for expression of an shRNA. The vector contains a template for transcription of an shRNA.

FIG. 8A shows a schematic diagram of a DNA cassette containing a U6 promoter and a template for transcription of an shRNA targeted to the HPRT protein.

FIG. 8B shows a schematic diagram of the predicted structure of a stem-loop RNA transcribed from a cassette such as that of FIG. 8A. It is noted that minor variations, e.g., in the number of bp in the stem, are generally not material and that the correspondence between FIGS. 8A and 8B in terms of number of base pairs of various elements may not be exact.

FIG. 9A shows a schematic diagram of a DNA cassette containing a U6 promoter and a template for transcription of an shRNA targeted to the GFP protein.

FIG. 9B shows the predicted structure of a stem-loop RNA transcribed from a cassette such as that of FIG. 9A. It is noted that minor variations, e.g., in the number of base pairs in the stem, are generally not material and that the correspondence between FIGS. 9A and 9B may not be exact.

FIG. 9C shows a schematic outline of a method for inserting a template for transcription of an shRNA targeted to GFP into a vector containing a U6 promoter.

FIG. 10 shows a schematic diagram of a dual DNA cassette containing two U6 promoters, one of which drives transcription of an shRNA targeted to the GFP protein and the other of which drives transcription of an shRNA targeted to the HPRT protein.

FIG. 11 shows a schematic diagram of a GFP-based screen using DNA transfection of a GSE library to identify genetic suppressor elements that inhibit RNAi.

FIG. 12 is a diagram of the retroviral vector pLXSfi-puro.

FIG. 13 is a schematic diagram of a screen using a library of genetic suppressor elements to identify genes involved in RNAi. Suppression is achieved with antisense RNA molecules.

FIG. 14 is a schematic diagram of a screen using a library of genetic suppressor elements to identify genes involved in RNAi. Screening is performed using detection of GFP fluorescence.

FIG. 15 is a schematic diagram of a screen using a library of genetic suppressor elements to identify genes involved in RNAi. Selection is performed by culturing cells in HAT medium.

FIG. 16 is a schematic diagram of a screen using a library of genetic suppressor elements to identify genes involved in RNAi. The library is delivered to cells using retroviral infection, and selection is performed by culturing cells in HAT medium.

FIG. 17 is a schematic diagram of a screen using a library of genetic suppressor elements to identify genes involved in RNAi. The library is delivered to cells using retroviral infection, and screening is performed using detection of GFP fluorescence.

FIG. 18 is a schematic diagram of a screen using a library of genetic suppressor elements to identify genes involved in RNAi. The library is delivered to cells using retroviral infection. A first round of selection is performed by culturing cells in HAT medium. Screening is then performed using detection of GFP fluorescence. A second round of selection and screening is performed on positive GSEs.

FIG. 19 is a schematic overview of a high throughput screen to identify chemical activators or inhibitors of RNAi pathways.

FIG. 20A shows flow cytometry results illustrating GFP fluorescence in a parental cell line, CHOk1-GFP that expresses GFP and different degrees of silencing of GFP in 12 clonal CHOk1-GFP-shGFP cell lines derived from the parental cell line and expressing a shRNA targeted to the GFP protein.

FIG. 20B shows quantitative flow cytometry results for the parental cell line CHOk1-GFP that expresses GFP and two clonal cell lines derived from the parental cell line and expressing a shRNA targeted to GFP.

FIG. 21 shows flow cytometry results demonstrating that shRNAs targeted to the Dicer enzyme reduce silencing of a detectable marker (GFP) by the RNAi pathway.

FIG. 22A is a bar graph showing a growth rate comparison of HeLa cells grown in DMEM (black bars) or in DMEM containing 8-AZ (lighter bars, on the right in each set of two adjacent bars). HeLa cells were mock transfected (1) or transfected with the following: siRNA targeted to HPRT (2), the antisense strand of the HPRT-targeted siRNA (3), the sense strand of the HPRT-targeted siRNA (4), or a non-specific siRNA as a control (5). Growth rate is expressed in terms of doublings per day.

FIGS. 22B-22D show photomicrographs of HeLa cells grown in DMEM (22B), DMEM containing 8-AZ (22C) or transfected with siRNA targeted to HPRT and grown in DMEM-containing 8-AZ (22D).

FIG. 22E shows results of a reverse transcription (RT)-polymerase chain reaction (PCR) of HPRT and β-actin (control) mRNAs derived from untransfected HeLa cells grown in DMEM (1) or grown in DMEM-containing 8-AZ (2) or from HeLa cells transfected with siRNA targeted to the HPRT gene and grown in 8-AZ (3). The (−) denotes the no RT control.

FIG. 23A is a bar graph showing a comparison of cellular growth rates of three CHO cell lines expressing different amounts of HPRT in F-12 media (black bars) or F-12 media containing 6-TG (lighter bars, on the right in each set of two adjacent bars). The two bars on the left (1) represent cell line CHOk1 (wild type HPRT expression). The two middle bars (2) represent cell line 5A9 (low HPRT expression). The two bars on the right (3) represent cell line A563 (no detectable HPRT expression). Growth rate is expressed in terms of doublings per hour. Divisions on the y-axis are 0.005, 0.01, 0.015, 0.02, 0.025, 0.03. FIG. 23B is a graph showing a time course of cell number for three CHO cell lines with different levels of HPRT expression grown in F-12 medium containing 6-TG as a function of time. The graph also shows the growth of a CHO cell line (CHO-shHPRT) that stably expresses an shRNA targeted to HPRT. Diamonds represent wild type CHOk1 cells. Squares represent 5A9 cells, a CHO cell line with a low level of HPRT expression. Triangles represent A563 cells, a CHO cell line with undetectable HPRT expression. X represents a CHOk1 cell line that stably expresses an shRNA targeted to HPRT and therefore silences HPRT expression. The x-axis represents time. The y-axis represents cell count per well, for identical wells in a 6-well multiwell plate.

FIG. 24A is a bar graph showing a comparison of cellular growth rates of three CHO cell lines expressing different amounts of HPRT in DMEM (black bars) or in DMEM containing HAT (red or lighter bars, i.e., right bar in each set of two adjacent bars). The two bars on the left (1) represent cell line CHOk1 (wild type HPRT expression). The two middle bars (2) represent cell line 5A9 (low HPRT expression). The two bars on the right (3) represent cell line A563 (no detectable HPRT expression). Growth rate is expressed in terms of doublings/hour.

FIG. 24B is a graph showing a time course of cell number in three CHO cell lines with different levels of HPRT expression grown in F-12 medium containing HAT as a function of time. The graph also shows the growth of a CHO cell line (CHOk1-shHPRT) that stably expresses an shRNA targeted to HPRT. Diamonds represent wild type CHOk1 cells. Squares represent 5A9 cells, a CHO cell line with a low level of HPRT expression. Triangles represent A563 cells, a CHO cell line with undetectable HPRT expression. X represents a CHOk1 cell line that stably expresses a shRNA targeted to HPRT and therefore silences HPRT expression. The x-axis represents time. The y-axis represents cell count per well, for identical wells in a 6-well multiwell plate.

FIG. 25 is a bar graph showing a comparison of cellular growth rates of wild type CHO cells expressing HPRT (black bars) and wild type CHO cells that stably express an shRNA targeted to the HPRT gene and lack HPRT expression (red or lighter bars, on the right in each set of two adjacent bars), under various selection conditions. Both wild type cells and cells expressing the HPRT hairpin RNA grow in F-12 medium (left bars). Cells that silence HPRT display a growth advantage in F-12 medium containing 6-TG relative to cells that express HPRT (middle bars). Cells that express HPRT live in medium containing HAT while cells that silence HPRT die in HAT-containing medium (right bars). Growth rate is expressed in terms of doublings/hour.

FIG. 26 (lower panels) shows growth of CHOk1-shHPRT cells expressing an shRNA targeted to HPRT in F-12 medium plus HAT in the presence of increasing concentrations of the putative inhibitor of RNAi, 5′AMPS. The preliminary data shown in these images suggests that increasing concentrations of the compound allow cells to grow in the presence of HAT. The upper panels show growth of wild type CHOk1 cells in F-12 medium in the absence (left) or presence (right) of 5′AMPS. Numbers represent concentration of 5′AMPS in the medium in μg/ml. Photos were taken after 4 days of growth in the presence of varying concentrations of 5′AMPS.

FIG. 27A shows the structure of adenosine 5′ O-thiomonophosphate (5′AMPS).

FIG. 27B shows the structure of a compound that was found to inhibit RNAi in a preliminary screen.

FIG. 28 illustrates a potential mechanism by which false positives may occur in a selection for compounds that inhibit RNAi and thus allow expression of HRPT. AMP can be converted to IMP. Compounds such as adenine and others that can be converted to AMP by adenine phosphoribosyltransferase (APRT) may thus allow cells expressing an shRNA targeted to HPRT to grow in HAT medium even if HPRT expression remains inhibited.

FIG. 29 shows the structure of AICA riboside.

FIGS. 30A and 30B show the structure and sequence of two precursors of endogenous human microRNAs, miR-30 (FIG. 30A) and miR-21 (FIG. 30B). Sequences of the mature miRNAs are underlined. Note the presence of several mismatches and/or bulges in the precursor structures. From Zeng, Y. and Cullen, B. R., RNA, 9:112-123, 2003.

FIG. 31A shows the structure of a nucleic acid encoding a detectable marker that was used to generate a cell line that can be used for identification of genes and/or chemical agents that affect the miRNA translational repression pathway. The construct encodes firefly luciferase upstream of 6 binding sites for the miR-21 miRNA. The figure represents the partial structure of a DNA construct or an mRNA transcribed therefrom.

FIG. 31B shows the duplex structure formed by binding of the miR-21 miRNA to its binding site in an mRNA transcript. Note the bulge typical of duplex structures formed by hybridization of an miRNA to its target.

FIG. 32 is a time course showing luciferase activity in a cell line containing the reporter construct shown in FIG. 31. The graph shows that inhibition of Drosha, a gene involved in the miRNA translational repression pathway that encodes an RNase III-like enzyme critical for production of miRNAs, in a cell line expressing the reporter construct shown in FIG. 31 results in increased expression of luciferase, thus demonstrating that the miRNA translational repression pathway is involved in silencing expression of the reporter.

FIG. 33A is a concentration titration in which 2′O-Me-modified RNA complementary to miR-21 was transfected into cells containing the luciferase reporter construct depicted in FIG. 31. The graph shows that miR-21 is specifically involved in silencing of the luciferase reporter construct.

FIG. 33B is a time course in which 2′O-Me-modified RNA complementary to miR-21 was transfected into cells containing the luciferase reporter construct depicted in FIG. 31. The graph shows that miR-21 is specifically involved in silencing of the luciferase reporter.

### DEFINITIONS

Antibody: In general, the term “antibody” refers to an immunoglobulin, which may be natural or wholly or partially synthetically produced in various embodiments of the invention. An antibody may be derived from natural sources (e.g., purified from a rodent, rabbit, chicken (or egg) from an animal that has been immunized with an antigen or a construct that encodes the antigen) partly or wholly synthetically produced. An antibody may be a member of any immunoglobulin class, including any of the human classes: IgG, IgM, IgA, IgD, and IgE. The antibody may be a fragment of an antibody such as an Fab′, F(ab′)₂, scFv (single-chain variable) or other fragment that retains an antigen binding site, or a recombinantly produced scFv fragment, including recombinantly produced fragments. See, e.g., Allen, T., Nature Reviews Cancer, Vol. 2, 750-765, 2002, and references therein. Preferred antibodies, antibody fragments, and/or protein domains comprising an antigen binding site may be generated and/or selected in vitro, e.g., using techniques such as phage display (Winter, G. et al. 1994. Annu. Rev. Immunol. 12:433-455, 1994), ribosome display (Hanes, J., and Pluckthun, A. Proc. Natl. Acad. Sci. USA. 94:4937-4942, 1997), etc. In various embodiments of the invention the antibody is a “humanized” antibody in which for example, a variable domain of rodent origin is fused to a constant domain of human origin, thus retaining the specificity of the rodent antibody. It is noted that the domain of human origin need not originate directly from a human in the sense that it is first synthesized in a human being. Instead, “human” domains may be generated in rodents whose genome incorporates human immunoglobulin genes. See, e.g., Vaughan, et al., Nature Biotechnology, 16: 535-539, 1998. An antibody may be polyclonal or monoclonal, though for purposes of the present invention monoclonal antibodies are generally preferred.

Approximately: As used herein, the term approximately in reference to a number is generally taken to include numbers that fall within a range of 5% in either direction of (i.e., greater than or less than) the number unless otherwise stated or otherwise evident from the context (except where such number would exceed 100% of a possible value). Where ranges are stated, the endpoints are included within the range unless otherwise stated or otherwise evident from the context. Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Complementary: The term complementary is used herein in accordance with its art-accepted meaning to refer to the capacity for precise pairing via hydrogen bonds (e.g., Watson-Crick base pairing or Hoogsteen base pairing) between two nucleosides, nucleotides or nucleic acids, etc. The hydrogen bonds exist between the purine and pyrimidine base moieties that form parts of the nucleotides. For example, if a nucleotide at a certain position of a first nucleic acid is capable of stably hydrogen bonding with a nucleotide located opposite to that nucleotide in a second nucleic acid, when the nucleic acids are aligned in opposite 5′ to 3′ orientation (i.e., in anti-parallel orientation), then the nucleic acids are considered to be complementary at that position (where position may be defined relative to either end of either nucleic acid, generally with respect to a 5′ end). The nucleotides located opposite one another may be referred to as a “base pair”. A complementary base pair contains two complementary nucleotides, e.g., A and U, A and T, G and C, etc., while a noncomplementary base pair contains two noncomplementary nucleotides (also referred to as a mismatch). Two nucleic acids are said to be complementary to each other when a sufficient number of corresponding positions in each molecule are occupied by nucleotides that hydrogen bond with each other, i.e., a sufficient number of base pairs are complementary.

Concurrent administration: As used herein with respect to two or more agents, e.g., therapeutic agents, concurrent administration is administration performed using doses and time intervals such that the administered agents are present together within the body, or at a site of action in the body such as within the eye) over a time interval in less than de minimis quantities. The time interval can be minutes, hours, days, weeks, etc. Accordingly, the agents may, but need not be, administered together as part of a single composition. In addition, the agents may, but need not be, administered simultaneously (e.g., within less than 5 minutes, or within less than 1 minute) or within a short time of one another (e.g., less than 1 hour, less than 30 minutes, less than 10 minutes, approximately 5 minutes apart). According to various embodiments of the invention agents administered within such time intervals may be considered to be administered at substantially the same time. One of ordinary skill in the art will be able to readily determine appropriate doses and time interval between administration of the agents so that they will each be present at more than de minimis levels within the body or, preferably, at effective concentrations within the body. When administered concurrently, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

Effective amount: An effective amount of an active agent refers to the amount of the active agent sufficient to elicit a desired biological response. As will be appreciated by those of ordinary skill in the art, the absolute amount of a particular agent that is effective may vary depending on such factors as the desired biological endpoint, the agent to be delivered, the target cell or tissue, etc. Those of ordinary skill in the art will further understand that an effective amount may be administered in a single dose, or may be achieved by administration of multiple doses.

Endogenous: An entity such as a gene or an expression product thereof, is considered endogenous to a cell if it is naturally present within the cell in the absence of modification of the cell, or an ancestor of the cell, by the hand of man. It will be appreciated that the amount of an endogenous RNA (and thus of a protein encoded by the RNA) present within a cell can be increased above its naturally occurring level by introducing a template for transcription of the RNA, operably linked to appropriate regulatory elements, into the cell. As applied to genes, RNAs, proteins, etc., the term endogenous is generally understood to refer to genes, RNAs, proteins, etc., as they naturally exist within a cell, unless otherwise indicated.

Gene: For the purposes of the present invention, the term “gene” has its meaning as understood in the art. In general, a gene is taken to include gene regulatory sequences (e.g., promoters, enhancers, etc.) and/or intron sequences, in addition to coding sequences (open reading frames). It will further be appreciated that definitions of “gene” include references to nucleic acids that do not encode proteins but rather encode functional RNA molecules, or precursors thereof, such as microRNA or siRNA precursors, tRNAs, etc. For the purpose of clarity it is noted that, as used in the present application, in most cases the term “gene” refers to a nucleic acid that includes a protein-coding portion; the term may optionally encompass regulatory sequences. However, this definition also encompasses application of the term “gene” to non-protein coding expression units

Gene product or expression product: A “gene product” or “expression product” is, in general, an RNA transcribed from the gene (e.g., either pre- or post-processing) or a polypeptide encoded by an RNA transcribed from the gene (e.g., either pre- or post-modification).

Hybridize: The term hybridize, as used herein, refers to the interaction between two complementary nucleic acid sequences in which the two sequences remain associated with one another under appropriate conditions. The phrase hybridizes under high stringency conditions describes an interaction that is sufficiently stable that it is maintained under art-recognized high stringency conditions. Guidance for performing hybridization reactions can be found, for example, in Current Protocols in Molecular Biology, John Wiley & Sons, N.Y., 6.3.1-6.3.6, 1989, and more recent updated editions, all of which are incorporated by reference. See also Sambrook, Russell, and Sambrook, Molecular Cloning: A Laboratory Manual, 3^{rd} ed., Cold Spring Harbor Laboratory Press, Cold Spring Harbor, 2001. Aqueous and nonaqueous methods are described in that reference and either can be used. Typically, for nucleic acid sequences over approximately 50-100 nucleotides in length, various levels of stringency are defined, such as low stringency (e.g., 6×sodium chloride/sodium citrate (SSC) at about 45° C., followed by two washes in 0.2×SSC, 0.1% SDS at least at 50° C. (the temperature of the washes can be increased to 55° C. for medium-low stringency conditions)); medium stringency (e.g., 6×SSC at about 45° C., followed by one or more washes in 0.2×SSC, 0.1% SDS at 60° C.); high stringency (e.g., 6×SSC at about 45° C., followed by one or more washes in 0.2×SSC, 0.1% SDS at 65° C.); and very high stringency (e.g., 0.5M sodium phosphate, 0.1% SDS at 65° C., followed by one or more washes at 0.2×SSC, 1% SDS at 65° C.) Hybridization under high stringency conditions only occurs between sequences with a very high degree of complementarity. One of ordinary skill in the art will recognize that the parameters for different degrees of stringency will generally differ based upon various factors such as the length of the hybridizing sequences, whether they contain RNA or DNA, etc. For example, appropriate temperatures for high, medium, or low stringency hybridization will generally be lower for shorter sequences such as oligonucleotides than for longer sequences.

Isolated: As used herein, isolated means 1) separated from at least some of the components with which it is usually associated in nature; 2) prepared or purified by a process that involves the hand of man; 3) not occurring in nature; and/or 4) not present as an integral part of an organism.

Ligand: As used herein, a ligand is a molecule that specifically binds to a second molecule, typically a polypeptide or portion thereof, such as a carbohydrate moiety, through a mechanism other than an antigen-antibody interaction. The term encompasses, for example, polypeptides, peptides, and small molecules, either naturally occurring or synthesized, including molecules whose structure has been invented by man. Although the term is frequently used in the context of receptors and molecules with which they interact and that typically modulate their activity (e.g., agonists or antagonists), the term as used herein applies more generally.

MicroRNA: A microRNA (miRNA) is a naturally occurring single-stranded RNA molecule that is naturally derived by processing of an endogenous precursor RNA containing a hairpin structure. MicroRNA precursors are typically transcribed from RNA Pol II promoters and, in some cases, are processed from introns present within Pol II-dependent genes. The miRNA forms a hybrid with a target site in a target transcript and reduces expression of the target transcript by translational repression, i.e., it blocks or prevents translation. In most cases, multiple binding sites (e.g., 3-6 sites) for a particular miRNA are present in the target transcript, usually in the 3′ UTR.

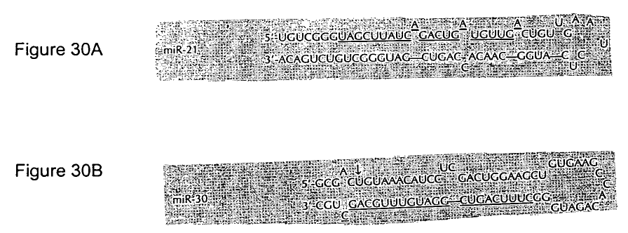

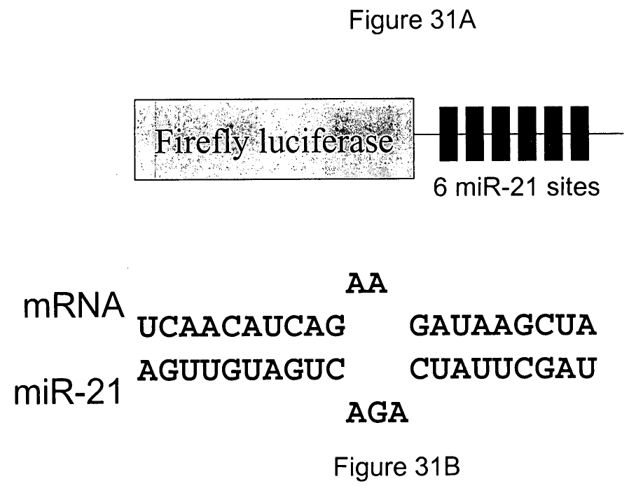

The hybrid formed between the miRNA and the target transcript is usually imperfect and typically contains one or more bulges. For purposes of the present invention, a “bulge” in a nucleic acid duplex structure is a region located between two complementary portions of the structure, in which either (i) at least two consecutive noncomplementary base pairs exist; or (ii) one of the strands includes one or more “extra” unpaired nucleotide(s) located between two regions of perfect base pair complementarity (i.e., unpaired regions of the two strands differ in the number of nucleotides they contain). In the latter case, one or more of the nucleotide(s) located 5′ to the extra nucleotide(s) and/or one or more of the nucleotide(s) located 3′ to the extra nucleotide(s) may be complementary or noncomplementary to the base pairs located opposite in the other strand. Preferably the bulge is located near the center of the duplex formed between the miRNA and its target transcript. It is generally preferred that any nucleotides 2-8 of the miRNA are perfectly complementary to the target. FIGS. 30A and 30B show the structure and sequence of precursors of two endogenous human miRNAs. Note the presence of several mismatches and/or bulges in the precursor structures. A large number of endogenous human miRNAs have been identified (Lagos-Quintana, M., et al, RNA, 9(2):175-9, 2003). See Bartel, D., supra, for structures of a number of endogenous miRNA precursors from various organisms. FIG. 31B shows the duplex structure formed by binding of the miR-21 miRNA to its binding site in an miRNA. Note that the duplex contains, i.e., is interrupted by, a bulge.

While the term “miRNA” is usually used to refer to endogenous RNAs that are naturally expressed, similar molecules or precursors thereof that either mimic the sequence of naturally occurring miRNAs or are specifically designed to hybridize to a target transcript so as to result in a duplex structure containing one or more bulges can be introduced into, and expressed within, cells and can cause translational repression. Thus either double-stranded duplex molecules structurally similar or identical to siRNAs, or hairpin precursors that can be processed intracellularly in a similar manner to naturally occurring miRNA precursors, can be introduced into cells and can mediate RNAi via translational repression (See, e.g., Doench, J., et al., Genes and Dev., 17:438-442, 2003). An RNAi-inducing entity that mediates RNAi by repressing translation of a target transcript, and that consists of or comprises a strand that binds to a target transcript to form a duplex containing one or more bulges, is said herein to act via an miRNA translational repression pathway, and the strand that binds to the target may be referred to as an miRNA-like molecule. A binding site with which a small, single-stranded RNA can hybridize to form a duplex structure containing a bulge, such that the transcript containing the binding site (or multiple copies thereof), is subject to RNAi via translational repression, is referred to herein as an miRNA binding site. Endogenous miRNAs can also mediate cleavage of RNA targets (i.e., they can act in an siRNA-like manner) if they have sufficient complementarity to the target. Further description of miRNAs and the mechanism by which they are believed to mediate silencing is found in Bartel, D., Cell, 116:281-297, 2004.

Nucleic acid, polynucleotide or oligonucleotide: These terms are generally used herein in their art-accepted manners to refer to a polymer of nucleotides. As used herein, an oligonucleotide is typically less than 100 nucleotides in length. A polynucleotide or oligonucleotide may also be referred to as a nucleic acid. Naturally occurring nucleic acids include DNA and RNA. Typically, a polynucleotide comprises at least three nucleotides. A nucleotide comprises a nitrogenous base, a sugar molecule, and a phosphate group. A nucleoside comprises a nitrogenous base linked to a sugar molecule. In a polynucleotide or oligonucleotide, phosphate groups covalently link adjacent nucleosides to form a polymer. The polymer may include natural nucleosides (e.g., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), and/or nucleosides comprising chemically or biologically modified bases, (e.g., methylated bases), intercalated bases, and/or modified sugars (e.g., 2′-fluororibose, ribose, 2′-deoxyribose, arabinose, and hexose). The phosphate groups in a polynucleotide or oligonucleotide are typically considered to form the internucleoside backbone of the polymer. In naturally occurring nucleic acids (DNA or RNA), the backbone linkage is via a 3′ to 5′ phosphodiester bond. However, polynucleotides and oligonucletides containing modified backbones or non-naturally occurring internucleoside linkages can also be used in the present invention. Such modified backbones include ones that have a phosphorus atom in the backbone and others that do not have a phosphorus atom in the backbone. Examples of modified linkages include, but are not limited to, phosphorothioate and 5′-N-phosphoramidite linkages. See U.S. Patent Application No. 20040092470 and references therein for further discussion of various nucleotides, nucleosides, and backbone structures that can be used in the polynucleotides or oligonucleotides described herein, and methods for producing them. Further information is also found elsewhere herein.

Polynucleotides and oligonucleotides need not be uniformly modified along the entire length of the molecule. For example, different nucleotide modifications, different backbone structures, etc., may exist at various positions in the polynucleotide or oligonucleotide. Any of the polynucleotides described herein may utilize these modifications.

Operably linked: As used herein, this term refers to a relationship between two nucleic acid sequences wherein the expression of one of the nucleic acid sequences is controlled by, regulated by, modulated by, etc., the other nucleic acid sequence. For example, the transcription of a nucleic acid sequence is directed by an operably linked promoter sequence; post-transcriptional processing of a nucleic acid is directed by an operably linked processing sequence; the translation of a nucleic acid sequence is directed by an operably linked translational regulatory sequence; the transport or localization of a nucleic acid or polypeptide is directed by an operably linked transport or localization sequence; and the post-translational processing of a polypeptide is directed by an operably linked processing sequence. Preferably a nucleic acid sequence that is operably linked to a second nucleic acid sequence is covalently linked, either directly or indirectly, to such a sequence, although any effective three-dimensional association is acceptable.

Purified: As used herein, purified means separated from one or more other compounds or entities. A compound or entity may be partially purified, substantially purified, or pure, where it is pure when it is removed from substantially all other compounds or entities (e.g., other compounds or entities in which it is found in nature), i.e., is preferably at least about 90%, more preferably at least about 91%, 92%, 93%, 94%, 95%, 96%, 97%, 98%, 99%, or greater than 99% pure.

Regulatory sequence: The term regulatory sequence is used herein to describe a region of nucleic acid sequence that directs, increases, or inhibits the expression (particularly transcription, but in some cases other events such as splicing or other processing) of sequence(s) with which it is operatively linked. The term includes promoters, enhancers and other transcriptional control elements. In some embodiments of the invention, regulatory sequences may direct constitutive expression of a nucleotide sequence; in other embodiments, regulatory sequences may direct tissue-specific and/or inducible expression. For instance, non-limiting examples of tissue-specific promoters appropriate for use in mammalian cells include lymphoid-specific promoters (see, for example, Calame et al., Adv. Immunol. 43:235, 1988) such as promoters of T cell receptors (see, e.g., Winoto et al., EMBO J. 8:729, 1989) and immunoglobulins (see, for example, Banerji et al., Cell 33:729, 1983; Queen et al., Cell 33:741, 1983), and neuron-specific promoters (e.g., the neurofilament promoter; Byrne et al., Proc. Natl. Acad. Sci. USA 86:5473, 1989). Developmentally-regulated promoters are also encompassed, including, for example, the murine hox promoters (Kessel et al., Science 249:374, 1990) and the α-fetoprotein promoter (Campes et al., Genes Dev. 3:537, 1989). In some embodiments of the invention regulatory sequences may direct expression of a nucleotide sequence only in cells that have been infected with an infectious agent. For example, the regulatory sequence may comprise a promoter and/or enhancer such as a virus-specific promoter or enhancer that is recognized by a viral protein needed for expresssion, e.g., a viral polymerase, transcription factor, etc.

RNAi-inducing agent: As used herein, in various embodiments of the invention the term RNAi-inducing agent refers to an RNA molecule or a vector (other than naturally occurring molecules not modified by the hand of man) whose presence within a cell results in RNAi and leads to reduced expression of a transcript to which the RNAi-inducing agent is targeted. In various embodiments of the invention an RNAi-inducing agent is an siRNA or shRNA. In other embodiments of the invention, rather than being an interfering RNA, such as an siRNA or shRNA, the RNAi-inducing agent is, or provides a template for transcription of, an interfering RNA such as an siRNA or shRNA, or a template for transcription of a precursor of an siRNA or shRNA, which precursor is processed within the cell to produce an siRNA or shRNA. The template may form part of a larger nucleic acid molecule. In certain embodiments of the invention the RNAi-inducing agent is a microRNA (miRNA) (either a naturally occurring or designed miRNA-like RNA), an siRNA that acts via a miRNA pathway, a precursor of a microRNA, a precursor of an siRNA that acts via an miRNA translational repression pathway, a template for transcription of a precursor of a microRNA or miRNA-like RNA, or a template for transcription of an siRNA that acts via an miRNA translational repression pathway. In certain embodiments of the invention the RNAi-inducing agent is an RNAi-inducing vector. The use of the term “induce” is not intended to indicate that the agent activates or upregulates RNAi in general but rather to indicate that presence of the agent within a cell results in RNAi-mediated reduction in expression of a transcript to which the agent is targeted although the agent may also activate or upregulate RNAi in general.

RNAi-inducing vector: An RNAi-inducing vector is a vector whose presence within a cell results in synthesis of an RNAi-inducing agent, e.g., results in transcription of one or more RNAs that self-hybridize or hybridize to each other to form an interfering RNA such as an shRNA, siRNA, or precursor of an miRNA or miRNA-like RNA. In various embodiments of the invention this term encompasses plasmids, e.g., DNA vectors (whose sequence may comprise sequence elements derived from a virus), or viruses, (other than naturally occurring viruses or plasmids that have not been modified by the hand of man), whose presence within a cell results in production of one or more RNAs that self-hybridize or hybridize to each other to form an interfering RNA such as an shRNA or siRNA or a precursor of a miRNA or miRNA-like RNA. In general, the vector comprises a nucleic acid operably linked to expression signal(s) so that one or more RNA molecules that hybridize or self-hybridize to form an interfering RNA such as an siRNA or shRNA or a precursor of a miRNA or miRNA-like RNA are transcribed when the vector is present within a cell. Thus the vector provides a template for intracellular synthesis of the RNA or RNAs or precursors thereof. It is noted that the template can be provided in RNA form, e.g., by a retrovirus, and converted into DNA form within the cell. For purposes of inducing RNAi, presence of a viral genome in a cell (e.g., following fusion of the viral envelope with the cell membrane) is considered sufficient to constitute presence of the virus within the cell. In addition, for purposes of inducing RNAi, a vector is considered to be present within a cell if it is introduced into the cell, enters the cell, or is inherited from a parental cell, regardless of whether it is subsequently modified or processed within the cell. An RNAi-inducing vector is considered to be targeted to a transcript if presence of the vector within a cell results in production of one or more RNAs that hybridize to each other or self-hybridize to form an interfering RNA such as an siRNA or shRNA or an miRNA or miRNA-like RNA that is targeted to the transcript, i.e., if presence of the vector within a cell results in production of one or more siRNAs or shRNAs or miRNAs or miRNA-like RNAs targeted to the transcript.

Short interfering RNA (siRNA): An siRNA comprises an RNA duplex (double-stranded region) and optionally further comprises one or two single-stranded overhangs, e.g., 3′ overhangs. Preferably the duplex is approximately 19 basepairs long although lengths between 17 and 29 nucleotides can be used. An siRNA may be formed from two RNA molecules that hybridize together or may alternatively be generated from a single RNA molecule that includes a self-hybridizing portion. It is generally preferred that free 5′ ends of siRNA molecules have phosphate groups and free 3′ ends have hydroxyl groups. The duplex portion of an siRNA may, but typically does not, include one or more bulges containing one or more unpaired and/or mismatched nucleotides in one or both strands of the duplex or may contain one or more noncomplementary nucleotide pairs. One strand of an siRNA (referred to as the antisense strand) includes a portion that hybridizes with a target transcript. In certain preferred embodiments of the invention, one strand of the siRNA (the antisense strand) is precisely complementary with a region of the target transcript over at least about 17 nucleotides, preferably 19 nucleotides, meaning that the siRNA antisense strand hybridizes to the target transcript without a single mismatch (i.e., without a single noncomplementary base pair) over that length. In other embodiments of the invention one or more mismatches between the siRNA antisense strand and the targeted portion of the target transcript may exist. In most embodiments of the invention in which perfect complementarity is not achieved, it is generally preferred that any mismatches between the siRNA antisense strand and the target transcript be located at or near 3′ end of the siRNA antisense strand. For example, in certain embodiments of the invention nucleotides 1-9, 2-9, 2-10, and/or 1-10 of the antisense strand are perfectly complementary to the target.

Short hairpin RNA (shRNA): The term short hairpin RNA refers to an RNA molecule comprising at least two complementary portions hybridized or capable of hybridizing to form a double-stranded (duplex) structure sufficiently long to mediate RNAi (generally between approximately 17 and 29 nucleotides in length, typically at least 19 base pairs in length), and at least one single-stranded portion, typically between approximately 1 and 10 nucleotides in length that forms a loop connecting the two nucleotides that form the base pair at one end of the duplex portion. The duplex portion may, but typically does not, contain one or more bulges consisting of one or more unpaired nucleotides. As described further below, shRNAs are thought to be processed into siRNAs by the conserved cellular RNAi machinery. Thus shRNAs are precursors of siRNAs and are, in general, similarly capable of inhibiting expression of a target transcript. As mentioned above, similar hairpin structures, which may be referred to as miRNA precursors, can be processed to yield endogenous miRNAs or RNAs that behave like endogenous miRNAs in that they translationally repress a target transcript. The latter are referred to herein as miRNA-like RNAs.

Small molecule: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds.

Specific binding: As used herein, the term specific binding refers to an interaction between a target polypeptide (or, more generally, a target molecule) and a binding molecule such as an antibody, ligand, agonist, or antagonist. The interaction is typically dependent upon the presence of a particular structural feature of the target polypeptide such as an antigenic determinant or epitope recognized by the binding molecule. For example, if an antibody is specific for epitope A, the presence of a polypeptide containing epitope A or the presence of free unlabeled A in a reaction containing both free labeled A and the antibody thereto, will reduce the amount of labeled A that binds to the antibody. It is to be understood that specificity need not be absolute but generally refers to the context in which the binding is performed. For example, it is well known in the art that numerous antibodies cross-react with other epitopes in addition to those present in the target molecule. Such cross-reactivity may be acceptable depending upon the application for which the antibody is to be used. One of ordinary skill in the art will be able to select antibodies having a sufficient degree of specificity to perform appropriately in any given application (e.g., for detection of a target molecule, for therapeutic purposes, etc). It is also to be understood that specificity may be evaluated in the context of additional factors such as the affinity of the binding molecule for the target polypeptide versus the affinity of the binding molecule for other targets, e.g., competitors. If a binding molecule exhibits a high affinity for a target molecule that it is desired to detect and low affinity for nontarget molecules, the antibody will likely be an acceptable reagent for immunodiagnostic purposes. Once the specificity of a binding molecule is established in one or more contexts, it may be employed in other, preferably similar, contexts without necessarily re-evaluating its specificity.

Subject: As used herein, subject refers to an individual to whom an agent is to be delivered, e.g., for experimental, diagnostic, and/or therapeutic purposes. Preferred subjects are mammals, particularly domesticated mammals (e.g., dogs, cats, etc.), primates, or humans.

Targeted: An RNAi-inducing agent such as an siRNA or shRNA is considered to be targeted to a target transcript for the purposes described herein if 1) the stability of the target transcript is reduced in the presence of the siRNA or shRNA as compared with its absence; and/or 2) the siRNA or shRNA antisense strand shows at least about 90%, more preferably at least about 91%, 92%, 93%, 94%, 95%, 96%, 97%, 98%, 99%, or 100% precise sequence complementarity with the target transcript for a stretch of at least about 15, more preferably at least about 17, yet more preferably at least about 18 or 19 to about-21-23 nucleotides; and/or 3) one strand of the siRNA (the antisense strand) or one of the self-complementary portions of the shRNA hybridizes to the target transcript under stringent conditions for hybridization of small (<50 nucleotide) RNA molecules in vitro and/or under conditions typically found within the cytoplasm or nucleus of mammalian cells, e.g., at physiological pH, salt concentration, and temperature. Percent sequence complementarity over a nucleotide stretch may be determined by dividing the number of complementary base pairs by the total number of base pairs (complementary+noncomplementary).

A miRNA or miRNA precursor is considered to be targeted to a target transcript for the purposes described herein if 1) the translation of the target transcript is reduced in the presence of the miRNA as compared with its absence; and/or 2) the miRNA (or antisense strand of an miRNA precursor) shows at least about 80% sequence complementarity with the target transcript for a stretch of at least about 15, more preferably at least about 17, yet more preferably at least about 18 or 19 to about 21-23 nucleotides, except that the region of precise sequence complementarity is interrupted by a bulge; and/or 3) the miRNA or one of the self-complementary portions of the miRNA precursor hybridizes to the target transcript under stringent conditions for hybridization of small (<50 nucleotide) RNA molecules in vitro and/or under conditions typically found within the cytoplasm or nucleus of mammalian cells, e.g., at physiological pH, salt concentration, and temperature.

An RNA-inducing vector whose presence within a cell results in production of an RNAi-inducing agent such as an siRNA, shRNA, or miRNA that is targeted to a transcript is also considered to be targeted to the target transcript. Since the effect of targeting a transcript is to reduce or inhibit expression of the gene that directs synthesis of the transcript, an RNAi-inducing entity such as an siRNA, shRNA, or miRNA, etc., targeted to a transcript is also considered to target the gene that directs synthesis of the transcript even though the gene itself (i.e., genomic DNA) is not thought to interact with the RNAi-inducing agent or components of the cellular silencing machinery. Thus as used herein, an RNAi-inducing agent that targets a transcript is understood to target the gene that provides a template for synthesis of the transcript.

Treating: As used herein, treating can generally include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur.

Vector: In general, the term vector refers to a nucleic acid molecule capable of mediating entry of, e.g., transferring, transporting, etc., a second nucleic acid molecule into a cell. The transferred nucleic acid is generally linked to, e.g., inserted into, the vector nucleic acid molecule. A vector may include sequences that direct autonomous replication, or may include sequences sufficient to allow integration into host cell DNA. Useful vectors include, for example, plasmids (typically DNA molecules, although RNA plasmids are also known), cosmids, and viral vectors. As is well known in the art, the term viral vector may refer either to a nucleic acid molecule (e.g., a plasmid) that includes virus-derived nucleic acid elements that typically facilitate transfer or integration of the nucleic acid molecule (examples include retroviral or lentiviral vectors) or to a virus or viral particle that mediates nucleic acid transfer (examples include retroviruses or lentiviruses). As will be evident to one of ordinary skill in the art, viral vectors may include various viral components in addition to nucleic acid(s).

Expression vectors are vectors that include regulatory sequence(s), e.g., expression control sequences such as a promoter, sufficient to direct transcription of an operably linked nucleic acid. An expression vector comprises sufficient cis-acting elements for expression; other elements for expression can be supplied by the host cell or in vitro expression system. Such vectors typically include one or more appropriately positioned sites for restriction enzymes, to facilitate introduction of the nucleic acid to be expressed into the vector.

### DETAILED DESCRIPTION OF CERTAIN EMBODIMENTS OF THE INVENTION

### I. Introduction and Overview

The regulation of gene expression by small RNAs is a field of increasing biological importance. The discovery of post-transcriptional gene silencing (PTGS) in plants and the related process of RNA interference (RNAi) in animals suggests an endogenous pathway of gene regulation modulated by small RNAs in which gene expression is silenced post-transcriptionally by target mRNA degradation (reviewed in Sharp, P. A. 2001. RNA interference—2001. Genes Dev 15:485-490; and in Hutvagner, G., and P. D. Zamore. 2002. RNAi: nature abhors a double-strand. Curr Opin Genet Dev 12:225-232) or by translational repression of a target transcript. By understanding and using RNAi in mammalian cells (Novina, C. D., M. F. Murray, D. M. Dykxhoorn, P. J. Beresford, J. Riess, S. K. Lee, R. G. Collman, J. Lieberman, P. Shankar, and P. A. Sharp. 2002. siRNA-directed inhibition of HIV-1 infection. Nat Med 8:681-686), knockdown of the expression of any gene is likely to be possible, providing a way to determine its function (Paddison, P. J., and G. J. Hannon. 2002. RNA interference: the new somatic cell genetics? Cancer Cell 2:17-23).

RNAi offers the unprecedented opportunity to identify new drug targets and to define the function of mammalian genes, e.g., human genes, in both biological and disease processes. In plants and worms, PTGS and RNAi may perform the general function of silencing expression of mobile genetic elements. Infection of a plant with a recombinant plant virus carrying a copy of a plant gene produces small RNAs complementary to that gene (Voinnet, O., C. Lederer, and D. C. Baulcombe. 2000. A viral movement protein prevents spread of the gene silencing signal in Nicotiana benthamiana. Cell 103:157-167) resulting in the silencing of both the virally encoded and genomic copies of that gene (Ruiz, M. T., O. Voinnet, and D. C. Baulcombe. 1998. Initiation and maintenance of virus-induced gene silencing. Plant Cell 10:937-946). Furthermore, the small RNAs are capable of spreading to other parts of the plant and silencing specific gene expression in regions of the plant not infected with the virus (Voinnet, O., C. Lederer, and D. C. Baulcombe. 2000. A viral movement protein prevents spread of the gene silencing signal in Nicotiana benthamiana. Cell 103:157-167). Thus, small RNAs may constitute a primitive immune function in plants (Ratcliff, F. G., Harrison, B. D., Baulcombe, D. C. 1997. A similarity between viral defense and gene silencing in plants. Science 276:1558-1560; Ratcliff, F. G., S. A. MacFarlane, and D. C. Baulcombe. 1999. Gene silencing without DNA. RNA-mediated cross-protection between viruses. Plant Cell 11: 1207-1216; Covey. 1997. Plants combat infection by gene silencing. Nature 385:781-782). In worms, generation of siRNAs is implicated in transposon silencing (Ketting, R. F., T. H. Haverkamp, H. G. van Luenen, and R. H. Plasterk. 1999. Mut-7 of C. elegans, required for transposon silencing and RNA interference, is a homolog of Werner syndrome helicase and RNaseD. Cell 99:133-141; Tabara, H., M. Sarkissian, W. G. Kelly, J. Fleenor, A. Grishok, L. Timmons, A. Fire, and C. C. Mello. 1999. The rde-1 gene, RNA interference, and transposon silencing in C. elegans. Cell 99:123-132) and derepression of transposon movement in worms mutated in genes involve in RNAi pathways suggests a role for RNAi in genome surveillance.

While numerous genes have been implicated in RNAi in other eukaryotes, few genes have been implicated in mammalian RNAi. As discussed further below, an RNase III-like ribonuclease named Dicer has been implicated in the production of certain RNAi-inducing agents, e.g., siRNAs, in organisms capable of PTGS and RNAi (Hutvagner, G., J. McLachlan, A. E. Pasquinelli, E. Balint, T. Tuschl, and P. D. Zamore. 2001. A cellular function for the RNA-interference enzyme Dicer in the maturation of the let-7 small temporal RNA. Science 293:834-838; Bernstein, E., A. A. Caudy, S. M. Hammond, and G. J. Hannon. 2001. Role for a bidentate ribonuclease in the initiation step of RNA interference. Nature 409:363-366). In addition, the PAZ-PIWI-Domain (PPD) family of proteins contains members required for RNAi in plants, worms and flies (Schwarz, D. S., and P. D. Zamore. 2002. Why do miRNAs live in the miRNP? Genes Dev 16:1025-1031). However, it appears likely that not all PPD proteins are required for RNAi. For example, in C. elegans the PPD gene rde-1 is necessary for RNAi but its paralogs, alg-1 and alg-2, are apparently not required (Grishok, A., A. E. Pasquinelli, D. Conte, N. Li, S. Parrish, I. Ha, D. L. Baillie, A. Fire, G. Ruvkun, and C. C. Mello. 2001. Genes and mechanisms related to RNA interference regulate expression of the small temporal RNAs that control C. elegans developmental timing. Cell 106:23-34). Mammals have 8 PPD proteins, only 2 of which, eIF2C 1 and eIF2C2, have thus far been found to copurify with the RNA-induced silencing complex (RISC) in which cleavage of target mRNAs occurs (Martinez, J., A. Patkaniowska, H. Urlaub, R. Luhrmann, and T. Tuschl. 2002. Single-Stranded Antisense siRNAs Guide Target RNA Cleavage in RNAi. Cell 110:563). It is not clear what roles (if any) the other homologs play in RNAi. It is also likely that genes from other families are required for RNAi itself or for its regulation.

The inventors have recognized that modulating one or more genes involved in mediating RNAi (e.g., increasing or decreasing their expression or increasing or decreasing their functional activity) may be used to increase or decrease the efficacy of RNAi in a cell or organism, e.g., to control the extent to which an RNAi-inducing agent present in the cell or organism is able to inhibit expression of a target mRNA transcript. The inventors have further recognized that modulation of one or more genes involved in mediating RNAi may be accomplished through chemical means, e.g., by contacting a cell or organism with a compound that inhibits or activates a gene involved in RNAi.

In addition to its great interest from a scientific standpoint, the identification of genes involved in RNAi and the ability to control the efficacy of RNAi would have numerous applications. For example, while it is generally possible to identify one or more RNAi-inducing agents that reduce the expression of any given target, in some cases this requires testing of multiple different inhibitory RNA sequences since the ability of various RNAi-inducing agents targeted to different portions of a single transcript to mediate cleavage of the transcript has been shown to vary. In addition, the efficacy of RNAi has been shown to vary in different cell types. This may be due to differences in the RNAi pathway(s) in such cell types and/or differences in the ability of exogenously delivered RNAi-inducing agents to enter the cells. The ability to increase the overall efficacy of RNAi would facilitate the effective targeting of a wider range of genes and cell types and would reduce the trial and error that is sometimes required to identify an RNAi-inducing agent having the desired degree of efficacy. Increasing the efficacy of RNAi would greatly facilitate therapeutic applications of RNAi, particularly in contexts in which it may be difficult to deliver high levels of RNA to target cells or tissues in a subject. Thus compounds that activate RNAi may be administed concurrently with therapeutic RNAi-inducing agents (i.e., RNAi-inducing agents that treat or prevent a disease or clinical condition). Increasing the overall efficacy of RNAi may also allow smaller amounts of RNAi-inducing agents to be used, thereby reducing the likelihood of nonspecific side effects.

In addition, in certain instances it is desirable to use RNAi to reduce expression of a first transcript whose sequence is very similar to that of a second transcript, while not reducing expression of the second transcript. For example, it may be desired to reduce expression of one allele of a gene (e.g., a dominant allele conferring an undesired phenotype) while not affecting expression of the other allele. A number of diseases and conditions, e.g., cancer, can be caused by mutations such as single nucleotide substitutions in an otherwise normal cellular gene. It may be desired to inhibit expression of the mutated copy of the gene while leaving expression of the non-mutated copy essentially intact (Brummelkamp, T. R., Bernards, R., and Agami, R. 2002. Stable suppression of tumorigenicity by virus-mediated RNA interference. Cancer Cell 2(3):243-7). In cases such as these, in order to achieve specificity, the sequence of the RNAi-inducing agent must in general encompass the mutated nucleotide and display greater complementarity to the mutated allele than to the normal copy. Thus the sequence of the RNAi-inducing agent is constrained by the necessity to target the particular portion of the transcript containing the mutation, which may not be a portion of the transcript that can be most effectively targeted by RNAi. The ability to enhance the activity of the RNAi pathway would facilitate the use of RNAi to target specific portions of transcripts, wherein the portions would otherwise be refractory to RNAi.

The ability to reduce the activity of an RNAi pathway would also be very useful. For example, while likely roles for RNAi in plants and worms have been identified, the biological function(s) of RNAi in mammalian cells and organisms remains unclear. However, as with other mechanisms of gene regulation, it seems likely that alterations in RNAi will be found to play a part in human disease. By reducing the activity of the RNAi pathway, e.g., using compounds identified according to the inventive methods described herein, it will be possible to determine the role(s) of RNAi both in normal cellular processes and in development of disease. In addition, the ability to reduce the activity of the RNAi pathway would be useful in the context of therapeutic uses of RNAi, which are anticipated based, for example, on studies showing effective inhibition of viral replication and effective inhibition of tumor development in animal models. For example, it may be desirable to reduce the activity of the RNAi pathway in certain cells that would otherwise experience deleterious effects from the delivery of an RNAi-inducing agent to a subject. Thus compounds that inhibit RNAi may be administed concurrently with therapeutic RNAi-inducing agents (i.e., RNAi-inducing agents that treat or prevent a disease or clinical condition).

In order to develop a system for identification of genetic components of RNAi pathways and for identification of compounds that activate or inhibit RNAi in mammalian cells, the inventors have developed and tested a variety of mammalian cells and cell lines. In general, the cells of the invention comprise a nucleic acid that encodes a marker such as a selectable or detectable marker and further comprise one or more templates for transcription of an RNAi-inducing agent that reduces expression of the marker, i.e., is targeted to an mRNA that encodes the marker. In accordance with the invention, a manipulation that increases or decreases the efficacy of RNAi will decrease or increase the expression of the marker, respectively. Thus a manipulation that increases the efficacy of RNAi will increase the ability of the RNAi-inducing agent to reduce expression of the marker, e.g., by enhancing cleavage of the mRNA. Conversely, a manipulation that decreases the efficacy of RNAi will decrease the ability of the RNAi-inducing agent to reduce expression of the marker, e.g., by reducing cleavage of the mRNA. The manipulation may be, for example, mutating a gene an expression product of which is involved in RNAi, expressing a genetic suppressor element that inhibits a nucleic acid or polypeptide involved in RNAi within a cell, contacting a cell with a compound that increases or decreases RNAi, e.g., by activating or inhibiting a gene whose expression product is involved in RNAi, etc. According to certain of the inventive methods, cells in which RNAi is decreased or increased following a manipulation are identified, e.g. by selecting for cells in which expression of a selectable marker is increased by the manipulation, by selecting for cells in which expression of a selectable marker is decreased by the manipulation, by screening for cells in which expression of a detectable marker is increased by the manipulation, or by screening for cells in which expression of a selectable marker is decreased by the manipulation.

To facilitate a better understanding of the invention the following section describes various RNAi pathways and molecules that mediate RNAi.

### II. RNAi Pathways and Molecules

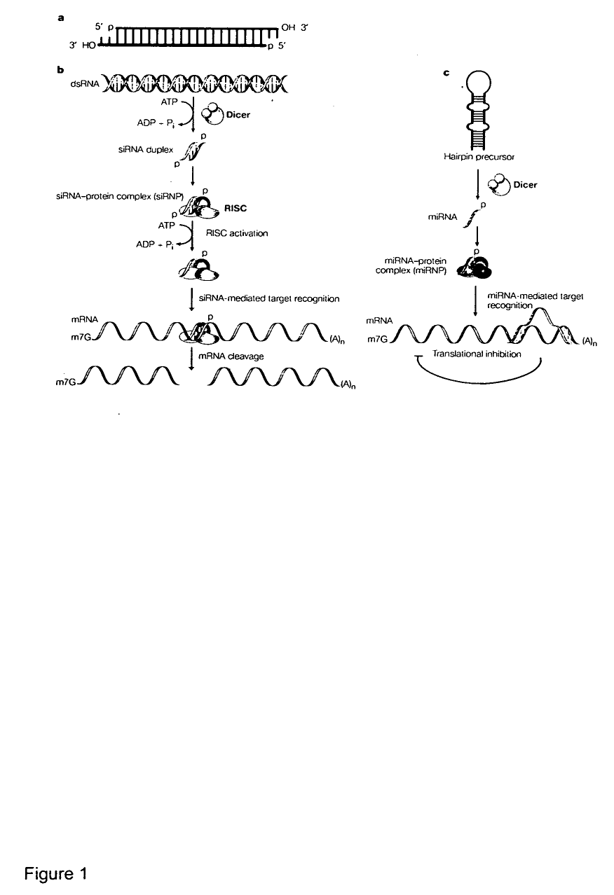

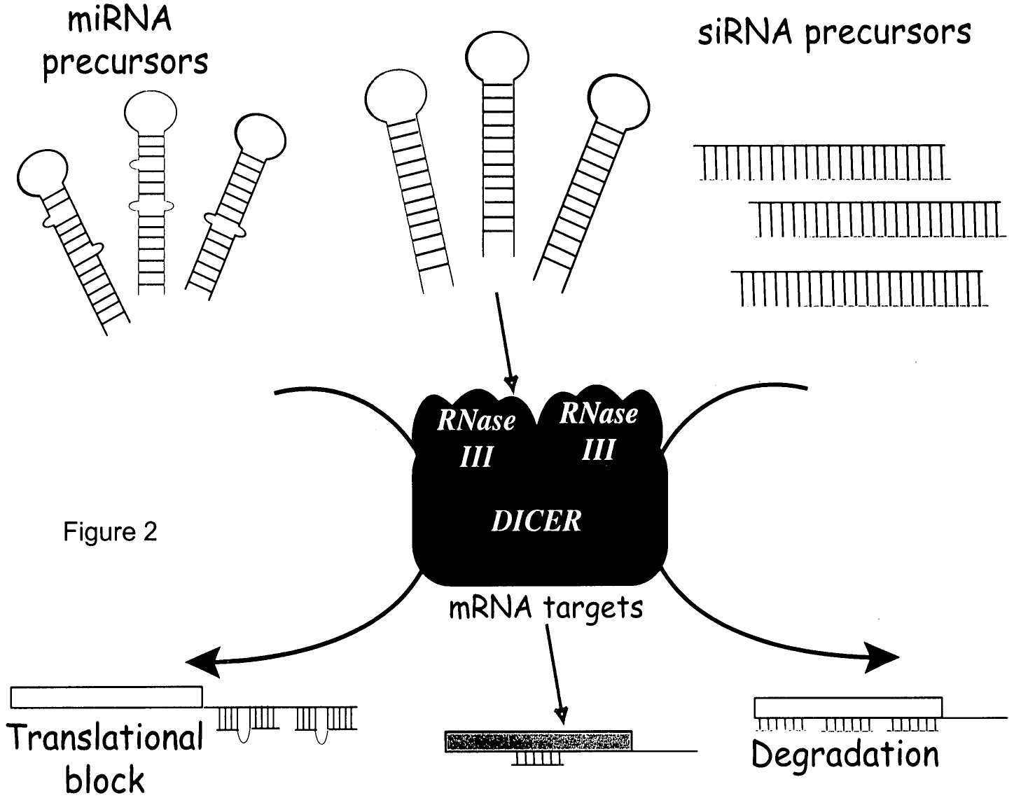

As mentioned above, short interfering RNAs (siRNAs) were discovered in studies of the phenomenon of RNA interference (RNAi) in Drosophila, as described in WO 01/75164 and WO02/44321. In particular, it was found that, in Drosophila, long double-stranded RNAs are processed by the RNase III-like enzyme Dicer (Bernstein et al., Nature 409:363, 2001) into smaller dsRNAs comprised of two 21-23 nt strands, each of which has a 5′ phosphate group and a 3′ hydroxyl, and includes a 19-21 nt region precisely complementary with the other strand, so that there is a 19-21 nt duplex region flanked by 2 nt-3′ overhangs. FIG. 1A presents a schematic diagram of such a short dsRNA (referred to as a short interfering RNA (siRNA).

FIG. 1B is a schematic diagram of the RNAi pathway by which dsRNAs, siRNAs, and precursors thereof such as shRNAs (see below), silence gene expression. The pathway begins with cleavage of a dsRNA molecule by Dicer in an ATP-dependent reaction that generates one or more siRNA molecules. As shown schematically in FIG. 1B, siRNAs act to silence expression of any gene that includes a region complementary to one of the siRNA strands, presumably because a helicase activity unwinds the duplex in the siRNA, allowing an alternative duplex to form between one strand of the siRNA and the target transcript. Thus one strand of the siRNA molecule (the antisense strand) is complementary to a target mRNA transcript transcribed from the gene. The antisense strand of the siRNA guides an endonuclease-containing complex known as the RNA induced silencing complex (RISC), to the portion of the target RNA that is complementary to the antisense strand. RISC then cleaves (“slices”) the target transcript at a single location, producing unprotected RNA ends that are promptly degraded by cellular machinery.

The finding of Dicer homologs in diverse species ranging from C. elegans to humans (Sharp, Genes Dev. 15;485, 2001; Zamore, Nat. Struct. Biol. 8:746, 2001) raised the possibility that an RNAi-like mechanism might be able to silence gene expression in a variety of different cell types including mammalian cells, e.g., human, cells. However, long dsRNAs (e.g., dsRNAs having a double-stranded region longer than about 30-50 nucleotides) are known to activate the interferon response in mammalian cells. Thus, rather than achieving the specific gene silencing observed with the Drosophila RNAi mechanism, the presence of long dsRNAs in mammalian cells would be expected to lead to interferon-mediated non-specific suppression of translation, potentially resulting in cell death. Long dsRNAs are therefore not thought to be useful for inhibiting expression of particular genes in mammalian cells.

However, siRNA molecules such as that depicted in FIG. 1A are able to effectively silence expression of target genes in mammalian cells without triggering an interferon response. Such molecules can be delivered exogenously to cells, thereby bypassing the cleavage step catalyzed by Dicer, e.g., by using standard methods useful for introducing DNA into mammalian cells such as cationic lipid-mediated transfection, electroporation, etc., or can be expressed intracellularly as discussed further below. While it is generally preferred that the antisense strand of the siRNA is perfectly complementary to the target transcript in order to obtain maximum silencing, siRNA molecules in which one or more mismatches exist between the antisense siRNA strand and the target transcript may also be effective, though generally less so than when perfect complementarity exists. In addition, while preferred siRNA molecules generally comprise a duplex portion of approximately 19-21 nucleotides in length, siRNAs having shorter or longer duplex portions may also be effective, though preferably such duplex portions are shorter than ˜30 nt in order to avoid triggering the interferon response. Preferred siRNA molecules typically include a 2 nt 3′ overhang on one or both strands though shorter or longer overhangs are also suitable. Considerations for design of effective siRNA molecules are discussed in McManus, M. and Sharp, P., Nature Reviews Genetics, 3: 737-747, and in Dykxhoorn, D. M., et al., Nature Reviews Molecular Cell Biology, 4: 457-467, 2003. Such considerations include the base composition of the siRNA, the position of the portion of the target transcript that is complementary to the antisense strand of the siRNA relative to the 5′ and 3′ ends of the transcript, etc. A variety of computer programs are also available to assist with selection of siRNA sequences, e.g., from Ambion (web site having URL www.ambion.com), at web site having URL www.sinc.sunysb.edu/Stu/shilin/rnai.html, etc. Additional design considerations that may also be employed are described in Semizarov, D., et al., Proc. Natl. Acad. Sci., Vol. 100, No. 11, pp. 6347-6352.

In addition to siRNAs having a structure such as that depicted in FIG. 1A, various other double-stranded RNA molecules can induce RNAi and thus inhibit gene expression in mammalian cells. In particular, RNA molecules having a hairpin (stem-loop) structure can be processed intracellularly by Dicer to yield an siRNA structure such as that depicted in FIG. 1A. These RNA molecules, referred to as short hairpin RNAs (shRNAs), contain two complementary regions that hybridize to one another (self-hybridize) to form a double-stranded (duplex) region referred to as a stem, a single-stranded loop connecting the nucleotides that form the base pair at one end of the duplex, and optionally an overhang, e.g., a 3′ overhang. Preferably, the stem is approximately 19-21 bp long, though shorter and longer stems (e.g., up to approximately 29 nt) may also be used. Preferably the loop is approximately 1-20, more preferably approximately 4-10, and most preferably approximately 6-9 nt long. Preferably the overhang, if present, is approximately 1-20, and more preferably approximately 2-10 nt long. One of ordinary skill in the art will appreciate that loops of 4 nucleotides or greater are less likely subject to steric constraints than are shorter loops and therefore may be preferred. The loop may be located at either the 5′ or 3′ end of the region that is complementary to the target transcript whose inhibition is desired (i.e., the antisense portion of the shRNA). In some embodiments, the shRNA includes a 5′ phosphate and a 3′ hydroxyl. shRNAs can be delivered exogenously or synthesized intracellularly as described further below.

Although shRNAs contain a single RNA molecule that self-hybridizes, it will be appreciated that the resulting duplex structure may be considered to comprise sense and antisense strands or portions relative to the target mRNA and may thus be considered to be double-stranded. It will therefore be convenient herein to refer to sense and antisense strands, or sense and antisense portions, of an shRNA, where the antisense strand or portion is that segment of the molecule that forms or is capable of forming a duplex with and is complementary to the targeted portion of the target transcript, and the sense strand or portion is that segment of the molecule that forms or is capable of forming a duplex with the antisense strand or portion and is substantially identical in sequence to the targeted portion of the target transcript. In general, considerations for selection of the sequence of the antisense strand of an shRNA molecule are similar to those for selection of the sequence of the antisense strand of an siRNA molecule that targets the same transcript.

Additional mechanisms of silencing mediated by short RNA species (microRNAs) are also known (see, e.g., Ruvkun, G., Science, 294, 797-799, 2001; Zeng, Y., et al., Molecular Cell, 9, 1-20, 2002). MicroRNAs (miRNAs) are single-stranded RNA molecules that are incorporated into an miRNA-protein complex which then recognizes a portion of a target transcript, typically having partial sequence complementarity to the miRNA. While endogenous siRNAs have not been found in mammals, miRNAs have been cloned from a variety of different mammalian cell types (Mourelatos, Z., J. Dostie, S. Paushkin, A. Sharma, B. Charroux, L. Abel, J. Rappsilber, M. Mann, and G. Dreyfuss. 2002. miRNPs: a novel class of ribonucleoproteins containing numerous microRNAs. Genes Dev 16:720-728). Generally the portion of a transcript complementary to an miRNA is found within the 3′ UTR of a gene. Rather than leading to cleavage of the target transcript, miRNA-mediated gene silencing often occurs as a result of translational repression and inhibition of protein synthesis. FIG. 1C shows a schematic diagram of the miRNA translational repression pathway. MicroRNAs can be produced intracellularly by cleavage of larger ˜70 nt hairpin precursors such as that shown at the top of FIG. 1C. The stem portion of such molecules typically contains at least one area of noncomplementarity such as a nucleotide bulge or inner loop in one or both strands. MicroRNAs produced in vivo by cleavage of artificial miRNA precursors can be used to mediate RNAi (McManus, M. T., C. P. Petersen, B. B. Haines, J. Chen, and P. A. Sharp. 2002. Gene silencing using micro-RNA designed hairpins. RNA 8:842-850). In addition, siRNAs that contain an antisense strand that exhibits less than perfect complementarity to a target transcript, e.g., in which one or more bulges exist when the antisense strand is paired with the target, can silence expression via an miRNA-like mechanism, i.e, a mechanism involving translational repression. While a number of the genes involved in the miRNA translational repression pathway are also involved in silencing by the siRNA RNAi pathway (transcript cleavage), it is clear that the overlap is incomplete and that certain of the genes that are required for, or involved in, processes such as miRNA precursor processing and translational repression mediated by miRNAs are distinct from those involved in processing of siRNA precursors or transcript cleavage mediated by siRNAs. Without not wishing to be bound by any theory, it may be of particular interest to identify genes involved in miRNA translational repression pathways and/or chemical modulators of miRNA pathways since such pathways are endogenous and may have a role in normal developmental processes and in the occurrence of human disease. Therefore, the ability to manipulate these pathways would be of considerable medical relevance.

In summary, double-stranded RNA molecules having a variety of different structures can cause RNA interference in mammalian cells. For purposes of the present invention, a genetic or biochemical pathway in which presence of a double-stranded RNA molecule within a cell leads to sequence-specific inhibition of expression of a target transcript is referred to as an RNA interference pathway, where “double-stranded” refers to (i) a duplex structure that consists of two individual nucleic acids hybridized to one another or (ii) a single nucleic acid containing complementary regions that hybridize to form a duplex structure. In other words, in RNAi the inhibitory RNA itself is double-stranded prior to processing and/or interaction with the target transcript, as described herein. Thus RNAi is distinct from so-called “antisense” mechanisms that typically involve inhibition of a target transcript by a single-stranded oligonucleotide. See, e.g., Crooke, S. (ed.) “Antisense Drug Technology: Principles, Strategies, and Applications” (1^{st} ed), Marcel Dekker; ISBN: 0824705661; 1st edition (2001).

A gene whose expression product or products plays a role in the biochemical processes of RNAi (e.g., cleavage or other processing of a dsRNA molecule to form an siRNA, miRNA, or miRNA-like molecule, unwinding of an siRNA or hairpin duplex, recognition or cleavage of a target transcript, translational repression of a target transcript, etc.) is considered to be directly involved in an RNAi pathway. Expression products of such genes may, but need not, be found in a RISC or in an miRNA-protein complex and/or may, but need not, be found in physical association with Dicer. In addition, a gene whose expression product acts a transcription factor for transcription of such a gene or whose expression product is involved in processing an expression product of such a gene is considered to be indirectly involved in an RNAi pathway. A gene whose expression product acts as a transcription factor for synthesis of one or more strands of a dsRNA molecule is also considered to be indirectly involved in an RNAi pathway. The present invention provides reagents and methods for identification of genes that are involved in one or more RNAi pathways and for identification of compounds that modulate (e.g., increase or decrease) their level of expression or functional activity. It is noted that the discussion of mechanisms and the figures depicting them are not intended to suggest any limitations on the present invention. An RNAi pathway in which silencing occurs by a process that involves cleavage of a target transcript mediated by a short RNA that binds to a target transcript to form a duplex structure is referred to as an siRNA RNAi pathway. An RNAi pathway in which silencing occurs by a process that involves translational repression mediated by a short RNA that binds to a target transcript to form a duplex structure that contains one or more bulges (i.e., the double-strandedness is interrupted by a bulge) is referred to as an miRNA translational repression pathway.

The present invention makes use of a variety of different methods for generateing short RNAs that silence gene expression. Those of ordinary skill in the art will readily appreciate that RNAi-inducing agents may be prepared according to any available technique including, but not limited to chemical synthesis, enzymatic or chemical cleavage in vivo or in vitro, template transcription in vivo or in vitro, or combinations of the foregoing. As noted above, RNA-inducing agents may be delivered as a single RNA molecule including self-complementary portions (e.g., an shRNA that can be processed intracellularly to yield an siRNA), or as two strands hybridized to one another. For instance, two separate 21 nt RNA strands may be generated, each of which contains a 19 nt region complementary to the other, and the individual strands may be hybridized together to generate a structure such as that depicted in FIG. 1A.

Alternatively, each strand may be generated by transcription from a promoter, either in vitro or in vivo. For instance, a construct may be provided containing two separate transcribable regions, each of which generates a 21 nt transcript containing a 19 nt region complementary with the other. Alternatively, a single construct may be utilized that contains opposing promoters and terminators positioned so that two different transcripts, each of which is at least partly complementary to the other, are generated. Alternatively, an RNA-inducing agent may be generated as a single transcript, for example by transcription of a single transcription unit encoding self complementary regions. A template is employed that includes first and second complementary regions, and optionally includes a loop region connecting the portions. Such a template may be utilized for in vitro transcription or in vivo transcription (by which is meant transcription in a cell), with appropriate selection of promoter and, optionally, other regulatory elements, e.g., a terminator.

In vitro transcription may be performed using a variety of available systems including the T7, SP6, and T3 promoter/polymerase systems (e.g., those available commercially from Promega, Clontech, New England Biolabs, etc.). As will be appreciated by one of ordinary skill in the art, use of the T7 or T3 promoters typically requires an siRNA sequence having two G residues at the 5′ end while use of the SP6 promoter typically requires an siRNA sequence having a GA sequence at its 5′ end. Vectors including the T7, SP6, or T3 promoter are well known in the art and can readily be modified to direct transcription of siRNAs. When siRNAs are synthesized in vitro the strands may be allowed to hybridize before transfection or delivery to a subject. Those of ordinary skill in the art will appreciate that, where RNAi-inducing agents are to be generated in vivo, it is generally preferable that they be produced via transcription of one or more transcription units. The primary transcript may optionally be processed (e.g., by one or more cellular enzymes) in order to generate the final agent that accomplishes gene inhibition.

It will further be appreciated that appropriate promoter and/or regulatory elements can readily be selected to allow expression of the relevant transcription units in mammalian cells. It is noted that the term “expression” as used herein in reference to synthesis (transcription) of an RNAi-inducing agent does not imply translation of the transcribed RNA. In certain embodiments of the invention the promoter utilized to direct intracellular expression of one or more transcription units that provide template(s) for transcription of an RNAi-inducing agent is a promoter for RNA polymerase III (Pol III). Pol III directs synthesis of small transcripts that terminate upon encountering a stretch of 4-5 T residues in the template. Certain Pol III promoters such as the U6 or H1 promoters do not require cis-acting regulatory elements (other than the first transcribed nucleotide) within the transcribed region and thus are preferred according to certain embodiments of the invention since they readily permit the selection of desired siRNA sequences. In the case of naturally occurring U6 promoters the first transcribed nucleotide is guanosine, while in the case of naturally occurring H1 promoters the first transcribed nucleotide is adenine. (See, e.g., Yu, J., et al., Proc. Natl. Acad. Sci., 99(9), 6047-6052 (2002); Sui, G., et al., Proc. Natl. Acad. Sci., 99(8), 5515-5520 (2002); Paddison, P., et al., Genes and Dev., 16, 948-958 (2002); Brummelkamp, T., et al., Science, 296, 550-553 (2002); Miyagashi, M. and Taira, K., Nat. Biotech., 20, 497-500 (2002); Paul, C., et al., Nat. Biotech., 20, 505-508 (2002); Tuschl, T., et al., Nat. Biotech., 20, 446-448 (2002). Thus in certain embodiments of the invention, e.g., where transcription is driven by a U6 promoter, the 5-nucleotide of preferred siRNA sequences is G. In certain other embodiments of the invention, e.g., where transcription is driven by an H1 promoter, the 5′ nucleotide may be A. According to other embodiments of the invention a promoter for RNA polymerase I, e.g., a tRNA promoter, is used (McCown, M., et al. Virology, 313(2):514-24, 2003; Kawasaki, H., and Taira, K., Nucleic Acids Res., 31 (2):700-7, 2003

According to certain embodiments of the invention promoters for RNA polymerase II (Pol II) may be used as described, for example, in Xia, H., et al., Nat. Biotechnol., 20, pp. 1006-1010, 2002. As described therein, constructs in which a hairpin sequence is juxtaposed within close proximity to a transcription start site and followed by a polyA cassette, resulting in minimal to no overhangs in the transcribed hairpin, may be employed. In addition, miRNA precursors are typically transcribed from Pol II promoters. In certain embodiments of the invention tissue specific, cell type specific, or regulatable (e.g., inducible or repressible) Pol II promoters may be used, provided the foregoing requirements are met. In other embodiments of the invention a constitutive promoter is used.

Intracellular expression of constructs that provide templates for synthesis of RNAi-inducing agents such as siRNAs, shRNAs, miRNA precursors, etc., can desirably be accomplished by introducing the constructs into a vector, such as, for example, a DNA plasmid (which may be a DNA vector comprising viral sequences) or a virus vector, and introducing the vector into mammalian cells. (In general, when reference is made to introducing a vector or construct into a cell, it is to be understood that the vector or construct may have been introduced into an ancestor of the cell rather than into the cell itself.) Any of a variety of vectors may be selected. The present invention includes vectors containing transcription units for transcription of one or more RNAi-inducing agents such as siRNAs or shRNAs, as well as cells containing such vectors or otherwise engineered to contain transcription units for transcription of one or more RNAi-inducing agents.

Preferred viral vectors for use in the compositions to provide intracellular expression of RNAi-inducing agents such as siRNAs and shRNAs include, for example, retroviral vectors and lentiviral vectors (which are considered a subset of retroviral vectors). See, e.g., Lois, C., et al., Science, 295: 868-872, 2002, describing the FUGW lentiviral vector; Somia, N., et al. J. Virol. 74(9): 4420-4424, 2000; Miyoshi, H., et al., Science 283: 682-686, 1999; U.S. Pat. No. 6,013,516; Rubinson, D., et al, Nature Genetics, Vol. 33, pp. 401-406, 2003; Stewart, S. A., et al., RNA, 9(4):493-501, 2003; Devroe, E., and Silver, P. A. BMC Biotechnol, 2(1):15, 2002; Barton, G. M., and Medzhitov, R. Proc Natl Acad Sci USA 99:14943-14945,2002). It will be appreciated that where the retroviral vector is a virus rather than a virus-based DNA vector, the viral genome must undergo reverse transcription and second strand synthesis to produce DNA capable of directing RNA transcription. In addition, where reference is made herein to elements such as promoters, regulatory elements, etc., it is to be understood that the sequences of these elements are present in RNA form in the retrovirus. Furthermore, where a template for synthesis of an RNA is “provided by” RNA present in a retrovirus, it is understood that the RNA must undergo reverse transcription and second strand synthesis to produce DNA that can serve as a template for synthesis of RNA. Vectors that provide templates for synthesis of an RNAi-inducing agent such as an siRNA or shRNA are considered to provide the RNAi-inducing agent when introduced into cells in which such synthesis occurs.

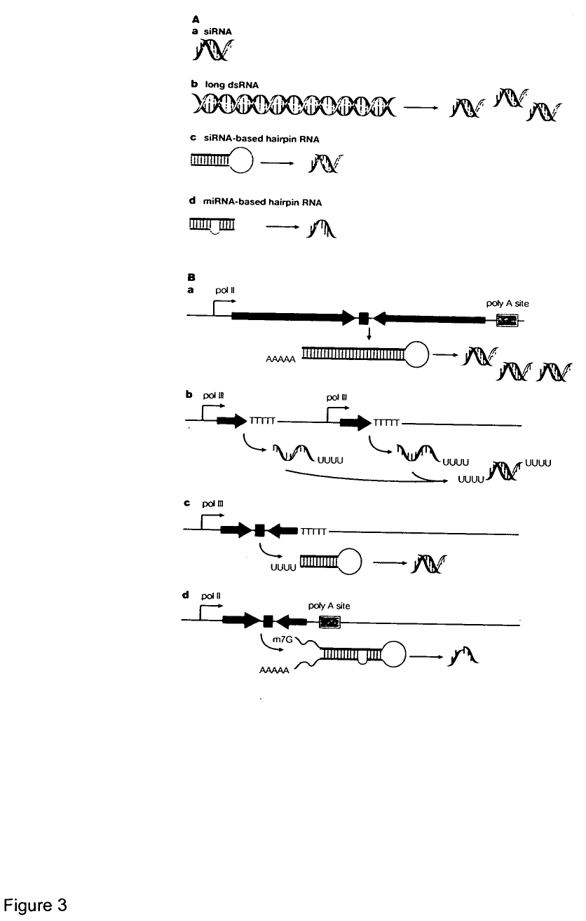

FIG. 3 summarizes a number of methods that can be used to generate RNAi-inducing agents such as those of the invention. FIG. 3A-a schematically depicts an siRNA, e.g., a chemically synthesized siRNA that can be introduced into cells (thereby bypassing the step of processing by Dicer) and incorporated into RISC for targeted messenger degradation. FIG. 3A-b shows a long dsRNA that can be introduced into cells and processed by Dicer into siRNAs that silence gene expression. This method will typically not be employed in mammalian cells or organisms with an intact interferon response. FIG. 3A-c shows a perfect duplex hairpin that can be cleaved by Dicer to yield siRNAs. Generally such hairpins contain duplex portions at least ˜19 bp in length. FIG. 3A-d shows an imperfect duplex hairpin RNA, designed based on naturally occurring pre-microRNA structures, that can be cleaved by Dicer to ultimately form miRNAs or siRNAs that act in a miRNA-like manner and direct gene silencing by a mechanism involving translational repression rather than mRNA cleavage. FIG. 3B-a shows a long hairpin RNA expressed from an RNA polymerase II promoter, which can be cleaved by Dicer to yield a population of siRNAs with different sequence specificities. This method will typically not be employed in mammalian cells or organisms with an intact interferon response. FIG. 3B-b shows production of a single siRNA using tandem RNA polymerase III promoters that express individual sense and antisense strands that associate in trans, e.g., within a cell. FIG. 3B-c shows production of a single siRNA using a single RNA polymerase III promoter that expresses a short hairpin RNA containing sense and antisense strands that associate in cis. FIG. 3B-d shows incorporation of an imperfect duplex hairpin structure designed based on naturally occurring pre-microRNA structures, which can be expressed from an RNA polymerase II promoter and processed by Dicer into a mature miRNA. A number of additional methods and variations on the above may also be used, some of which are described above. In general, the transcription cassettes depicted in FIGS. 3B-a through 3B-d may be inserted into vectors such as those mentioned above that can be introduced into cells for intracellular synthesis of RNAi-inducing agents. Such cassettes can be inserted into the genome of a cell, resulting in stable synthesis of an RNAi-inducing agent within the cell and its progeny.

As mentioned above, certain embodiments of the inventive methods may make use of synthetic RNAi-inducing agents, e.g., RNAi-inducing agents synthesized in vitro. In addition, certain embodiments of the invention such as the kits described below may include one or more synthetic RNAi-inducing agents such as siRNAs. It will be appreciated by those of ordinary skill in the art that such agents may be comprised entirely of natural RNA nucleotides, or may instead include one or more nucleotide analogs. A wide variety of such analogs is known in the art; the most commonly employed in studies of therapeutic nucleic acids being the phosphorothioate (for some discussion of considerations involved when utilizing phosphorothioates, see, for example, Agarwal, Biochim. Biophys. Acta 1489:53, 1999). In particular, it may be desirable to stabilize the RNA structure, for example by including nucleotide analogs at one or more free strand ends in order to reduce digestion, e.g., by exonucleases. The inclusion of deoxynucleotides, e.g., pyrimidines such as deoxythymidines, at one or more free ends may serve this purpose. Alternatively or additionally, it may be desirable to include one or more nucleotide analogs in order to increase or reduce stability of the stem, in particular as compared with any hybrid that will be formed by interaction of the antisense strand of an siRNA with a target transcript.

Various nucleotide modifications may be used selectively in either the sense or antisense strand of an siRNA or shRNA. For example, it may be preferable to utilize unmodified ribonucleotides in the antisense strand while employing modified ribonucleotides and/or modified or unmodified deoxyribonucleotides at some or all positions in the sense strand. Numerous nucleotide analogs and nucleotide modifications are known in the art, and their effect on properties such as hybridization and nuclease resistance has been explored. For example, various modifications to the base, sugar and internucleoside linkage have been introduced into oligonucleotides at selected positions, and the resultant effect relative to the unmodified oligonucleotide compared.

A number of modifications have been shown to alter one or more aspects of the oligonucleotide such as its ability to hybridize to a complementary nucleic acid, its stability, etc. For example, useful 2′-modifications include halo, alkoxy and allyloxy groups. U.S. Pat. Nos. 6,403,779; 6,399,754; 6,225,460; 6,127,533; 6,031,086; 6,005,087; 5,977,089, and references therein disclose a wide variety of nucleotide analogs and modifications that may be of use in the practice of the present invention. See also Crooke, S. (ed.) “Antisense Drug Technology: Principles, Strategies, and Applications” (1^{st} ed), Marcel Dekker; ISBN: 0824705661; 1st edition (2001) and references therein. As will be appreciated by one of ordinary skill in the art, analogs and modifications may be tested using, e.g., the assays described herein or other appropriate assays, in order to select those that effectively reduce expression of a target transcript. Certain desirable analog or modifications may result in an RNAi-inducing agent with increased entry into cells, increased absorbability (e.g., absorbability across a mucus layer, increased oral absorption, etc.), increased stability in the blood stream or within cells, increased ability to cross cell membranes, etc. As will be appreciated by one of ordinary skill in the art, analogs or modifications may result in altered Tm, which may result in increased tolerance of mismatches between the antisense sequence and the target while still resulting in effective suppression or may result in increased or decreased specificity for desired target transcripts.

In general, the ability of a candidate RNAi-inducing agent to reduce the level of the target transcript may be assessed directly by measuring the amount of the target transcript using, for example, Northern blots, nuclease protection assays, reverse transcription (RT)-PCR, real-time RT-PCR, microarray analysis, etc. The ability of a candidate siRNA to inhibit production of a polypeptide encoded by the target transcript (either at the transcriptional or post-transcriptional level) may be measured directly using a variety of affinity-based approaches (e.g., using a ligand or antibody that specifically binds to the polypeptide) including, but not limited to, Western blots, immunoassays, ELISA, flow cytometry, protein microarrays, etc. In general, any method of measuring the amount of either the target transcript or a polypeptide encoded by the target transcript may be used. For certain transcripts (e.g., transcripts that encode a detectable marker), the ability of a candidate RNAi-inducing agent to reduce the level of a target transcript may also be assessed indirectly, e.g., by measuring a functional activity of the polypeptide encoded by the transcript or by measuring a signal produced by the polypeptide encoded by the transcript.

A variety of methods can be used to assess the ability of an RNAi-inducing agent to reduce the level of a translation product of a target transcript. For example, if the translation product is a detectable marker it can be detected as appropriate for the particular marker, as described elsewhere herein. If the translation product is a selectable marker, it can be detected by determining the ability of cells to grow under either positive or negative selection, depending on the marker. Translation products may also be measured using a variety of methods for protein detection known in the art including, but not limited, to, immunologically based methods such as immunoblots, Elisa assays, etc. It will frequently be desirable to also measure transcript levels in order to confirm that an alteration in the level of a translation product is not due to increased or decreased transcript level or, if there is an increase or decrease in transcript level, that such increase or decrease is insufficient to account for the observed increase or decrease in the level of the translation product. It may also be desirable to confirm that an increase or decrease in the level of a translation product is due to an affect on an miRNA translational repression pathway rather than an affect on some component of the translational machinery that is not involved in miRNA-mediated translational repression. Comparisons with control cell lines may be made to confirm that a mutation, compound, etc., is actually affecting an RNAi pathway of interest.

### III. Cells, Cell Lines, and Vectors

Cells and Cell Lines

The present invention provides a variety of mammalian cells and cell lines that may be used to identify genes involved in an RNAi pathway and/or to identify compounds that modulate RNAi, e.g., that increase or decrease the effectiveness of an RNAi-inducing agent in inhibiting expression of a target transcript. In particular, the invention provides a mammalian cell comprising: (i) a nucleic acid that encodes a marker; and (ii) one or more nucleic acid templates for transcription of an RNAi-inducing agent integrated into the genome of the cell, wherein the RNAi-inducing agent reduces expression of the marker and is not naturally found in the cell. The cell may further comprise (i) a nucleic acid that encodes a detectable marker; and (ii) one or more templates for transcription of an RNAi-inducing agent that reduces expression of the detectable marker. Any of the foregoing cells may further comprise (i) a nucleic acid that encodes a selectable marker; and

(ii) one or more templates for transcription of an RNAi-inducing agent that reduces expression of the selectable marker. In general, the RNAi-inducing agent can be any of the agents discussed above suitable for expression in mammalian cells, e.g., an siRNA, shRNA, miRNA, or miRNA precursor. In the case of any of the cells and cell lines described herein, the nucleic acid that encodes a marker, e.g., a selectable or detectable marker, can be stably integrated into the genome of the cell, present within an episome (i.e., a genetic element that replicates in the cell independently of the genomic DNA), or can be present transiently in the cell, e.g., as a result of transient transfection. In various embodiments of the invention nucleic acids that provide templates for transcription of RNAi-inducing agents such as siRNAs, shRNAs, miRNAs, or miRNA-like RNAs can be stably integrated into the genome of the cell, present within an episome, or present transiently in the cell.

The inventive cells and cell lines can be used to identify genes involved in RNAi and/or compounds that modulate RNAi. In accordance with the invention, a loss of function mutation in a gene involved in the RNAi pathway by which the RNAi-inducing agent reduces expression of the marker will reduce or prevent RNAi-induced silencing of the marker so that the marker is expressed. Similarly, a compound that inhibits the RNAi pathway by which the RNAi-inducing agent reduces expression of the marker will reduce or prevent RNAi-induced silencing of the marker so that the marker is expressed. In certain embodiments of the invention cells in which the marker is expressed are identified by subjecting them to selection (in the case of a selectable marker whose expression confers a growth advantage on cells under selective conditions) or screening (e.g., by detecting a signal resulting from expression of the marker). Where the detectable marker is a bidirectional marker (e.g., where expression of the marker confers a growth advantage under a first set of selective conditions and a growth disadvantage under a second set of conditions), inactivity of the RNAi pathway can be confirmed by subjecting the cells to the second set of selective conditions. If RNAi is inactive, cells should grow under the second set of selective conditions.

According to the invention a gain of function mutation in a gene involved in the RNAi pathway by which the RNAi-inducing agent reduces expression of the marker will increase RNAi-induced silencing of the marker so that expression of the marker is decreased or absent. Similarly, a compound that activates or potentiates the RNAi pathway by which the RNAi-inducing agent reduces expression of the marker will enhance RNAi-induced silencing of the marker so that expression of the marker is reduced or inhibited entirely. In certain embodiments of the invention cells in which the marker is expressed are identified by subjecting them to selection (in the case of a selectable marker whose expression confers a growth disadvantage on cells under selective conditions) or screening, (e.g., by detecting lack of a signal resulting from expression of the marker, which can easily be done using FACS where the signal is fluorescence). Where the detectable marker is a bidirectional marker (e.g., where expression of the marker confers a growth advantage under a first set of selective conditions and a growth disadvantage under a second set of conditions), increased activity of the RNAi pathway can be confirmed by subjecting the cells to the second set of selective conditions. If RNAi activity is enhanced, cells should fail to grow or should grow less well under the second set of selective conditions than cells having wild type RNAi activity.

In certain embodiments of the invention the cells comprise a nucleic acid that encodes a selectable marker and a nucleic acid that encodes a detectable marker and also express one or more RNAi-inducing agents targeted to each marker. Thus cells in which RNAi is inhibited or activated can be identified using either selection or screening for expression of one of the markers and may then be retested using the other marker. Cells may also comprise nucleic acids encoding a plurality of selectable markers and express RNAi-inducing agents targeted to each of these markers. Double selection methods (e.g., imposing conditions that are selective for each of the markers) may be applied to cells. Cells may also comprise nucleic acids encoding a plurality of detectable markers and express RNAi-inducing agents targeted to each of these markers.

The invention further provides a mammalian cell comprising (i) a nucleic acid that is integrated into the genome of the cell and provides a template for transcription of an mRNA transcript that encodes a detectable or selectable marker, wherein the transcript comprises one or more binding sites for an endogenous miRNA or an miRNA-like RNA; and (ii) an endogenous miRNA, or an miRNA-like RNA, that is expressed by the cell and represses translation of the mRNA that encodes a detectable or selectable marker. The miRNA-like RNA may be an RNA that is designed to mimic a naturally occurring miRNA. In the latter case, a nucleic acid that provides a template for transcription of the miRNA-like RNA is introduced into the cell. The miRNA binding sites present in the mRNA transcript may either be naturally occurring binding sites for the endogenous mRNA or may be designed to mimic such sites. It will be appreciated that the binding site and the miRNA or miRNA-like RNA are selected in conjunction with one another, i.e., they are selected so that the miRNA or miRNA-like RNA will bind to the binding site to form a duplex structure that is interrupted by one or more bulges, as is the case for naturally occurring miRNA/binding site interactions. The ability of an miRNA or miRNA-like RNA to bind to a binding site and mediate translational repression of a transcript containing the site(s) may be readily tested as described in the Examples and known in the art. In certain embodiments of the invention the cell also contains an siRNA or siRNA precursor (e.g., an shRNA) targeted to the transcript. The antisense strand of the siRNA or shRNA is typically complementary to a different site to the miRNA binding site. Thus the transcript may be repressed by both siRNA RNAi pathway(s) and miRNA translational repression pathways. The invention also provides control cells and cell lines in which binding sites for the miRNA are not present in the mRNA transcript that encodes the marker, and thus the transcript is not translationally repressed (or, if any binding sites for the miRNA are present, they are not sufficient to result in translational repression by the miRNA). As described further below, the control cells may be used to distinguish mutations, compounds, or genetic elements that affect an miRNA translational repression pathway from mutations, compounds, or genetic elements that affect an siRNA RNAi pathway.

In addition, the invention provides a mammalian cell comprising (a) a nucleic acid that is integrated into the genome of the cell and provides a template for transcription of an mRNA transcript that encodes a first detectable or selectable marker; and (b) (i) a nucleic acid that is integrated into the genome of the cell and provides a template for transcription of an mRNA transcript that encodes a second detectable or selectable marker, wherein the transcript comprises one or more binding sites for an endogenous miRNA or an miRNA-like RNA; and (ii) an endogenous miRNA, or an miRNA-like RNA, that is expressed by the cell and represses translation of the mRNA that encodes a detectable or selectable marker. Preferably the transcript that encodes the first detectable or selectable marker does not contain binding sites for the endogenous miRNA or miRNA-like RNA for which the transcript that encodes the second marker has binding sites for, though it may contain binding sites for a different miRNA or miRNA-like RNA. The first and second detectable or selectable markers are generally different although they may be the same. In a preferred embodiment the first and second markers are distinguishable detectable markers, e.g., firefly and Renilla luciferase. The cell can be used, for example, to determine whether a compound, mutation, etc., that affect silencing affects siRNA pathways, miRNA translational repression pathways, or both.

Cells of the invention can be mutagenized or treated in a variety of ways, some of which are further described below, to cause loss of function or gain of function alterations in genes. The affected genes can then be identified and cloned. Populations of cells of the invention can be contacted with a plurality of compounds, e.g., compounds in a compound library, and compounds that affect the efficacy of RNAi can be identified.

In certain embodiments of the invention the cell is a human cell while in other embodiments of the invention the cell is a non-human cell, e.g., a rodent cell such as a mouse cell, hamster cell, etc. Suitable cells include, but are not limited to, HeLa cells, CHO cells, HEK-293, BHK, NIH/3T3, HT1080, COS, 293T, WI-38, and CV-1. For an extensive list of mammalian cell lines, those of ordinary skill in the art may refer to the American Type Culture Collection catalog. In general, the cells may be of any cell type, e.g., fibroblast, myoblast, T or B cell, macrophage, epithelial cells, etc.

In certain embodiments of the invention the cell is a hypodiploid cell, which means that the cell is structurally or functionally hemizygous at one or more loci, preferably at least 10% of loci, at least 25% of loci, at least 50% of loci, at least 75% of loci, or more. For example, CHO cells are known to be hypodiploid at many different loci (Gupta, R. S., et al., Cell 14:1007-1013, 1978; Gupta, R. S., et al., Cell. Physiol. 97:461-467, 1978. A number of hypodiploid human cell lines also exist, and monosomic cell lines (e.g., cell lines lacking one or more chromosomes) can be generated using methods known in the art (Clarke, D. J., Proc. Natl. Acad. Sci., 95(1): 167-171, 1998). Use of hypodiploid cells for purposes such as identifying genes involved in an RNAi pathway may be preferred because for hypodiploid loci it is only necessary to disable a single copy of the gene rather than two copies in order to identify recessive loss of function mutations in such cells.

The cell lines of the invention in general comprise a plurality of any of the cells of the invention or descendents thereof that can be maintained continously in culture over an extended period of time, typically months or years. Thus cells of any particular cell line are generally of the same cell type, same state of ploidy, will typically comprise the same nucleic acid that encodes a detectable marker and the same template(s) for transcription of an RNAi-inducing agent that reduces expression of the marker. In certain embodiments of the invention a cell line is derived from a single cell, e.g., by a single step cloning procedure, resulting in a clonal cell line. While cell lines that contain a heterogenous population of cells not derived from a single cell are not excluded, cell lines derived from single cells are generally preferred since, for example, they will typically display more uniform expression of the selectable and/or detectable marker(s) and/or more uniform degrees of RNAi.

As discussed in further detail in the Examples, clonal cell lines derived from the same parental cell line may display different degrees of RNAi-mediated gene silencing for reasons that remain unclear. Such cell lines are useful for different purposes. For example, it may be preferable to use a cell line in which RNAi-mediated gene silencing is highly active to identify a loss of function mutation in an RNAi pathway or a chemical activator of RNAi, while it may be preferable to use a cell line in which RNAi-mediated gene silencing is less active to identify a gain of function mutation in an RNAi pathway or a chemical inhibitor of RNAi. Accordingly, the invention provides a collection of cell lines wherein cells of each cell line comprise: (i) a nucleic acid that encodes a marker, wherein the nucleic acid in cells of each cell line encodes the same marker; and (ii) a template for transcription of an RNAi-inducing agent that reduces expression of the marker, wherein the RNAi-inducing agent reduces expression of the marker to different extents in cells of each of the cell lines.

Markers

In general, any polypeptide whose presence within the cell results in a detectable or otherwise identifiable phenotypic change in the cell can serve as a marker. Although the term “marker” will generally refer herein to a polypeptide encoded by an RNA, it will be appreciated that those of ordinary skill in the art also use the term to refer to a nucleic acid or gene that provides a template for transcription of a marker polypeptide and that frequently the distinction is not material. Where significant, whether the term “marker” is being used to refer to a polypeptide, a nucleic acid, or a gene will be clear from the context.

In general, a suitable marker for use in the invention is a detectable marker or a selectable marker. The term “selectable marker” is used herein as it is generally understood in the art and refers to a marker whose presence within a cell confers a significant growth or survival advantage or disadvantage on the cell under certain defined culture conditions (selective conditions). For example, the conditions may be the presence or absence of a particular compound or environmental condition such as increased temperature, increased radiation, etc. The presence or absence of such compound(s) or environmental condition(s) is referred to as a “selective condition” or “selective conditions”. By “growth advantage” is meant either enhanced viability (e.g., cells with the growth advantage have an increased life span, on average, relative to otherwise identical cells), increased rate of cell proliferation (also referred to herein as “growth rate”) relative to otherwise identical cells, or both. In general, a population of cells having a growth advantage will exhibit fewer dead or nonviable cells and/or a greater rate of cell proliferation that a population of otherwise identical cells lacking the growth advantage. Although typically a selectable marker will confer a growth advantage on a cell, certain selectable markers confer a growth disadvantage on a cell, e.g., they make the cell more susceptible to the deleterious effects of certain compounds or environmental conditions than otherwise identical cells not expressing the marker.

Antibiotic resistance markers are a non-limiting example of a class of selectable marker that can be used to select cells that express the marker. In the presence of an appropriate concentration of antibiotic (selective conditions), such a marker confers a growth advantage on a cell that expresses the marker. Thus cells that express the antibiotic resistance marker are able to survive and/or proliferate in the presence of the antibiotic while cells that do not express the antibiotic resistance marker are not able to survive and/or are unable to proliferate in the presence of the antibiotic. For example, a selectable marker of this type that is commonly used in mammalian cells is the neomycin resistance gene (an aminoglycoside 3′-phosphotransferase, 3′ APH II). Expression of this selectable marker renders cells resistant to various antibiotics such as G418. Additional selectable markers of this type include enzymes conferring resistance to zeocin™, hygromycin, puromycin, etc. These enzymes and the genes encoding them are well known in the art

A second non-limiting class of selectable markers is nutritional markers. Such markers are generally enzymes that function in a biosynthetic pathway to produce a compound that is needed for cell growth or survival. In general, under nonselective conditions the required compound is present in the environment or is produced by an alternative pathway in the cell. Under selective conditions, functioning of the biosynthetic pathway in which the marker is involved is needed to produce the compound.

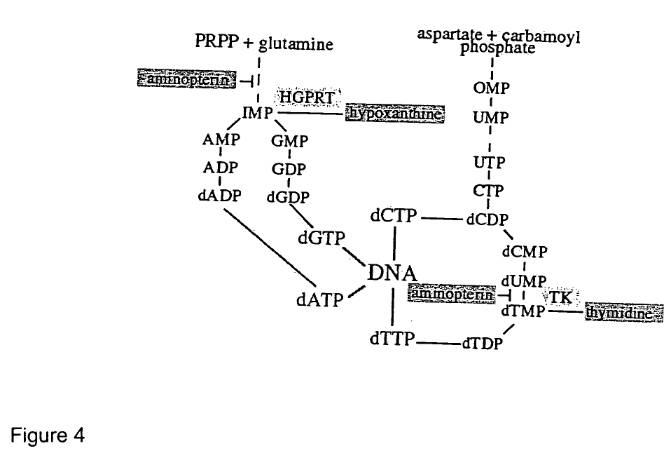

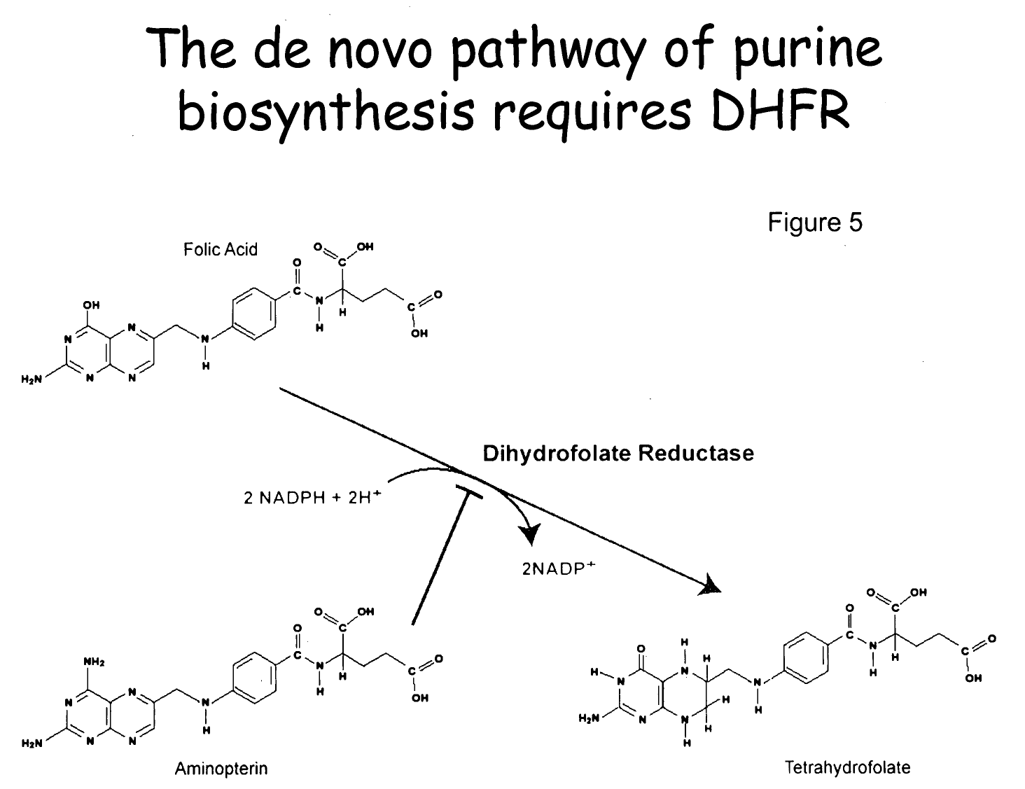

Two examples of such markers that are suitable for use in the invention are hypoxanthine phosphoribosyl transferase (HPRT), an enzyme that catalyzes certain reactions in which purine-type compounds are synthesized and/or interconverted, and thymidine kinase (TK), which catalyzes certain reactions in which pyrimidine-type compounds are synthesized and/or interconverted. Under typical culture conditions DNA synthesis in mammalian cells proceeds through a main (de novo) pathway in which glutamine and aspartate, respectively, are used as initial substrates for a series of reactions leading to synthesis of purine-type (e.g., dATP and dGTP) and pyrimidine-type (e.g., dCTP and dTTP) nucleotides. FIG. 4 presents an overview of these pathways. Another reaction required for function of the de novo nucleotide synthesis pathways is catalyzed by DHFR, is shown on FIG. 5. Inhibition of DHFR results in blockage of several of the reactions in the de novo pathway, as shown on FIG. 4. DHFR can be inhibited by a variety of compounds including aminopterin and methotrexate.

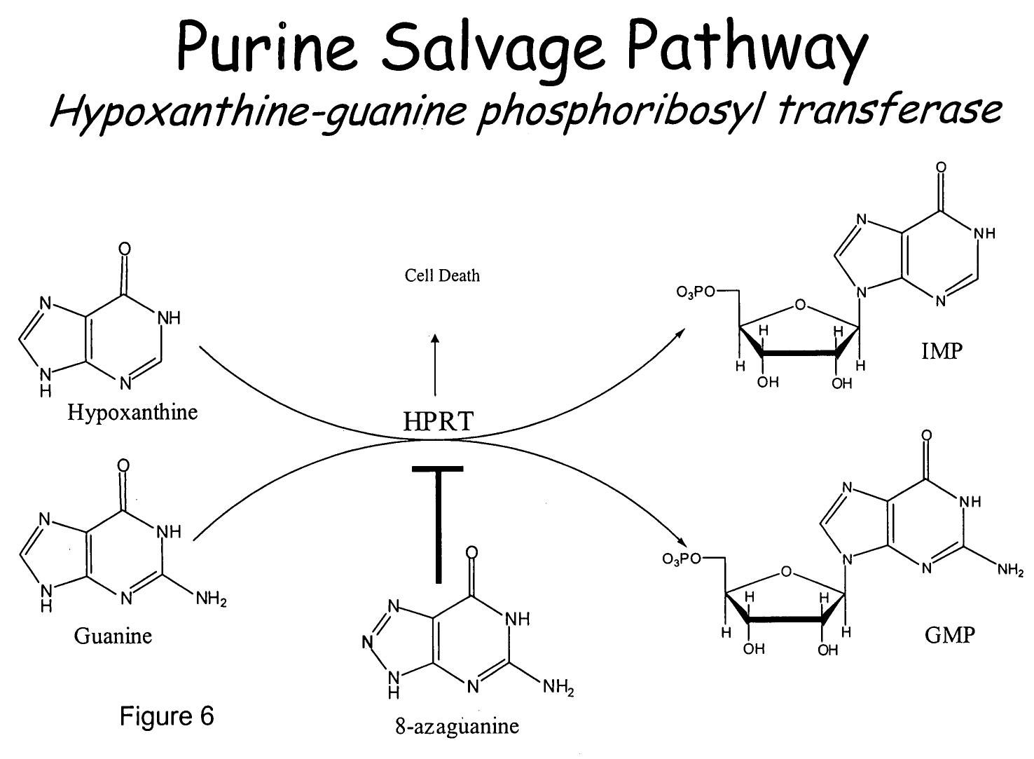

When the de novo pathway is blocked, e.g., due to inhibition of DHFR, mammalian cells must utilize alternative pathways to synthesize the needed nucleotides. The first of these pathways, known as the purine salvage pathway, converts hypoxanthine to IMP, a reaction which is catalyzed by HPRT, allowing synthesis of needed purine-type compounds (FIG. 6). The second pathway converts thymidine to dTMP, a reaction catalyzed by TK. Thus cells lacking HPRT expression (e.g., cells lacking a functional copy of the HPRT gene) or lacking TK expression (e.g., cells lacking a functional copy of the TK gene) can grow in standard culture medium but die in HAT medium, which contains aminopterin, hypoxanthine, and thymidine). In cells lacking HPRT expression, HPRT is a selectable marker whose presence may be selected for in HAT medium. Similarly, in cells lacking TK expression, TK is a selectable marker whose presence may be selected for in HAT medium.

In addition to the ability to select for cells that express HPRT or TK, it is also possible to select for cells that lack functional HPRT or TK, e.g., cells that do not express one or both of these enzymes. HPRT converts certain otherwise non-toxic compounds including a variety of purine analogs such as 8-azaguanine (8-AZ) and 6-thioguanine (6-TG) into cytotoxic compounds (FIG. 6). TK converts a variety of purine analogs such as 5-bromodeoxyuridine and trifluoro-methyl-thymidine into cytotoxic compounds. The cytotoxic compounds may have deleterious effects on cells through a variety of different mechanisms, e.g., they may inhibit enzymes involved in nucleic acid synthesis and/or become incorporated into DNA, leading to mismatches and mutations. Thus in culture medium containing 8-AZ, 6-TG, etc., cells that express HPRT will be at a growth disadvantage relative to cells that do not express HPRT or express it at lower levels. It is therefore possible to use these selective conditions to select for cells that lack HPRT activity. Similarly, in medium containing bromodeoxyuridine or trifluoro-methyl-thymidine cells that express TK will be at a growth disadvantage relative to cells that lack TK expression or express a lower level of TK. It is therefore possible to use these selective conditions to select for cells that lack TK activity. Thus in the case of certain selectable markers such as HPRT and TK, it is possible to select either for cells that express the marker or to select for cells that do not express the marker. Such selectable markers may be particularly preferred for use in the present invention.

A variety of additional selectable markers exist for which it is possible to select cells that do not express the marker. In general, selective conditions for this type of marker are deleterious for the cell in the presence of the marker but not in its absence. For example, selective conditions may result in synthesis of a cytotoxic compound or entry of a cytotoxic compound into a cell, etc., by a process that involves the selectable marker. Alternatively, expression of the marker may result in inhibition of a required biosynthetic pathway in the presence of the selective conditions It will be appreciated that markers of this type are preferably non-essential since otherwise it will generally not be possible to select cells that lack expression of the marker.

Additional examples of selectable markers that can be used to select cells that express the marker include proteins such as P-glycoprotein (MDR1) and related proteins known as multidrug resistance (MDR) proteins. These proteins act as pumps through which various compounds (e.g., chemotherapeutic agents such as vinblastine, anthracyclines, etc., which are used to treat cancer) are expelled from cells. (See Ambudkar S V, et al., Oncogene, 22(47):7468-85, 2003 for a review of these proteins). In the presence of a cytotoxic compound such as a chemotherapeutic agent, cells that express members of the MDR family are at a growth advantage relative to cells that do not express such proteins since the cells expressing an MDR family member pump out the cytotoxic compound and are therefore able to survive in its presence.

Examples of selectable markers that can be used to select cells that do not express the marker include proteins that act as channels or otherwise increase permeability of a cell to a cytotoxic agent or enhance activity of a cytotoxic agent.

Table 1 lists certain selectable markers and corresponding selective conditions that may be used in the context of the present invention either to select cells that express the marker (or that express it at a higher level than otherwise identical cells) or to select cells that do not express the marker (or that express it at a lower level than otherwise identical cells). Such markers are said to allow bidirectional selection. In cells that contain a template for transcription of an RNAi-inducing agent targeted to the marker, these markers allow for selection of cells in which RNAi is active or enhanced by selecting cells lacking expression of the marker and also allow for selection of cells in which RNAi is decreased or absent by selecting cells having expression of the marker. It will be appreciated that a variety of other selective conditions could be used for certain of the markers listed in Table 1 and that other markers are known to those of ordinary skill in the art. For example, selectable markers comprising fusion proteins such as TKneo, HyTK (hygromycin-TK), TKBSD, and puΔTK have been described, which can allow for bidirectional selection (Chen, Y., and Bradley, A., Genesis, 28:31-35, 2000).

TABLE 1
Selectable Markers Allowing Bidirectional Selection

| | Selective Condition to | Selective Condition |
|---|---|---|
| Selectable | Select Cells that | to Select Cells that |
| Marker | Do Not Express Marker | Express Marker |
| Hypoxanthine | 6-thioguanine; | HAT (hypoxanthine, |
| guanine | 8-azaguanine; | aminopterin, |
| phosphoribosyl- | 8-azahypoxanthine; | thymidine); HAS |
| transferase | 6-mercaptopurine | |
| (HPRT, HGPRT) | | |
| Thymidine | 5-bromodeoxyuridine; | HAT |
| kinase (TK) | ³H-thymidine; | |
| | trifluoro-methyl-thymidine; | |
| | gancyclovir; | |
| | 1-(-2 deoxy-2-fluoro-1-β-D- | |
| | arabinofuranosyl)-5-iodouracil | |
| Adenine | 2,6-diaminopurine; | AAT, |
| phosphoribosyl- | 8-azaadenine; | AAS, |
| transferase | 2-fluoroadenine | Alanosine + |
| (APRT) | | Adenine |
| Adenine kinase | 6-mercaptopurine riboside; | Adenosine + |
| | high adenosine | Methotrexate + |
| | | TdR + coformycin |
| Deoxycytidine | 5-bromodeoxycytidine | HA + CdR |
| deaminase | | |
| Deoxycytidine | cytosine arabinoside | High TdR + Cdr |
| kinase | | |

In summary, in certain embodiments of the invention the selectable marker confers a growth advantage on cells expressing the marker under a selective condition while in other embodiments of the invention the selectable markers confers a growth disadvantage on cells expressing the marker under the selective condition. In certain embodiments of the invention the selectable marker confers a growth advantage on cells expressing the marker under a first selective condition while in other embodiments of the invention the selectable markers confers a growth disadvantage on cells expressing the marker under a second selective condition. It will be appreciated that expression of certain oncogenes (e.g., genes that, when mutated or overexpressed result in cell transformation and/or tumor formation) or lack of expression of certain tumor suppressor genes (also referred to as recessive oncogenes) may confer a growth and/or survival advantage on cells. With respect to the cells, cell lines, and vectors of the invention selectable markers do not include proteins encoded by tumor suppressor genes (e.g., p53) and also do not include proteins encoded by oncogenes (e.g., Ras, Myc, Brc-Abl, etc.) However, such proteins may be used as selectable markers in the practice of the inventive methods.

In general, a detectable marker is a marker whose presence within a cell can be detected through means other than subjecting the cell to a selective condition or directly measuring the level of the marker itself. Thus in general, the expression of a detectable marker within a cell results in the production of a signal that can be detected and/or measured. The process of detection or measurement may involve the use of additional reagents and may involve processing of the cell. For example, where the detectable marker is an enzyme, detection or measurement of the marker will typically involve providing a substrate for the enzyme. Preferably the signal is a readily detectable signal such as light, fluorescence, luminescence, bioluminescence, chemiluminescence, enzymatic reaction products, or color. Thus preferred detectable markers for use in the present invention include fluorescent proteins such as green fluorescent protein (GFP) and variants thereof. A number of enhanced versions of GFP (eGFP) have been derived by making alterations such as conservative substitutions in the GFP coding sequence. Certain of these enhanced versions of GFP display increased fluorescence intensity or expression relative to wild type GFP and may be preferred. Certain other variants display decreased stability relative to wild type GFP. Such variants are referred to as destabilized. A particularly preferred detectable marker is a destabilized version of an enhanced GFP in which eGFP is fused to amino acid residues 422-461 of the mouse ornithine decarboxylase (MODC) protein. This C-terminal region of MODC contains a PEST amino acid sequence that targets the protein for degradation and results in rapid protein turnover.

In general, PEST sequences, also referred to as PEST domains, are protein regions rich in proline (P), glutamic/aspartic acid (E), serine (S), and threonine (T), that mark proteins containing them for intracellular proteolysis (Rogers, S., et al., Science 234, 364-368, 1986). These PEST regions are generally, but not always, flanked by clusters containing several positively charged amino acids. Addition of a PEST domain to a protein, e.g., by creating an N-terminal or C-terminal fusion or inserting a PEST domain elsewhere in the protein can lead to more rapid degradation of the protein. Thus modifying a protein (e.g., by modifying a nucleic acid that encodes the protein) so that the protein comprises a PEST domain will frequently result in a protein with decreased half-life. The invention encompasses the use of detectable markers such as destabilized eGFP that comprise a PEST domain in cis with the detectable marker sequence. The detectable marker may be a fusion protein having one or more PEST domains at either the N-terminus or the C-terminus. Alternately, the PEST domain may be present elsewherein the protein. Either a naturally occurring PEST domain (i.e., PEST domain having a sequence found in nature) or a synthetic PEST domain can be used. The invention also encompasses the use of detectable markers modified to include other sequences that result in increased degradation, e.g., a KFERQ sequence (Dice, J. F., Trends Biochem. Sci. 15, 305-309, 1990) or a cyclin destruction box (Glotzer, M., Murray, A. W., and Kirschner, M. W., Nature 349, 132-138, 1991). The ability of any particular protein domain to reduce the half-life of a protein can readily be tested using methods well known in the art. Preferably addition of the domain does not substantially reduce the maximum magnitude of the signal produced by the detectable marker.

Certain preferred detectable markers have a half-life of approximately 2 hours or less. Thus the invention provides a cell comprising: (i) a nucleic acid that encodes a detectable marker, wherein the detectable marker has a half-life of approximately 2 hours or less; and (ii) a template for transcription of an RNAi-inducing agent that reduces expression of the detectable marker integrated into the genome of the cell, wherein the RNAi-inducing agent reduces expression of the marker and is not naturally found in the cell. In certain embodiments of the invention the detectable marker has a half-life of approximately 1 hour or less. The invention further provides a cell comprising: (i) a nucleic acid that encodes a detectable marker, wherein the detectable marker comprises a domain that results in increased intracellular proteolysis of the marker relative to an otherwise identical marker lacking the domain; and (ii) a template for transcription of an RNAi-inducing agent that reduces expression of the detectable marker integrated into the genome of the cell, wherein the RNAi-inducing agent reduces expression of the marker and is not naturally found in the cell. In certain embodiments of the invention the detectable marker has a half-life of approximately 1 hour or less. In certain embodiments of the invention the domain is a PEST domain.

Other detectable markers that produce a fluorescent signal include red, blue, yellow, cyan, and sapphire fluorescent proteins, reef coral fluorescent protein, etc. A wide variety of such markers is available commercially, e.g., from BD Biosciences (Clontech). Additional detectable markers preferred in certain embodiments of the invention include luciferase derived from the firefly (Photinus pyralis) or the sea pansy (Renilla reniformis), as mentioned above. In addition, a detectable signal can be a detectable alteration in a biological pathway or response to an agent, e.g., a chemical agent. In certain embodiments of the invention a version of a marker whose coding sequence has been altered to optimize the use of codons for expression in mammalian cells is used.

Vectors

The invention provides a number of vectors that can be used in the construction of the inventive cells or for other purposes. In general, any suitable mammalian expression vector can be used to introduce a nucleic acid that encodes a selectable or detectable marker into cells, and any of a variety of art-recognized methods for introducing such vectors into cells can be used (e.g., DNA transfection using calcium phosphate, DEAE-dextran, cationic lipids, etc., viral infection in the case of virus vectors, etc.). Numerous vectors containing promoters functional in mammalian cells, such as the cytomegalovirus (CMV) promoter, herpes simplex virus (HSV) promoter, SV40 promoters, retroviral LTR, etc. are available. In the case of certain markers such as HPRT or TK, cells may already express the marker.

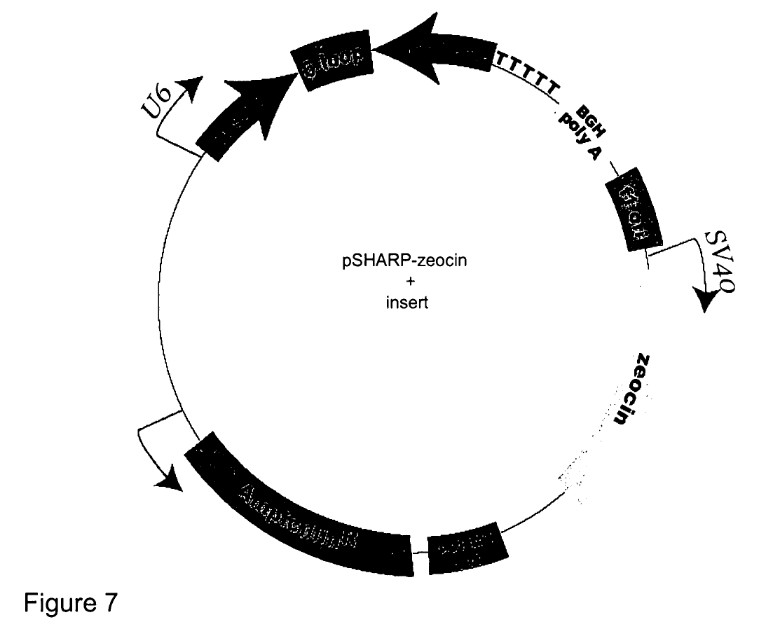

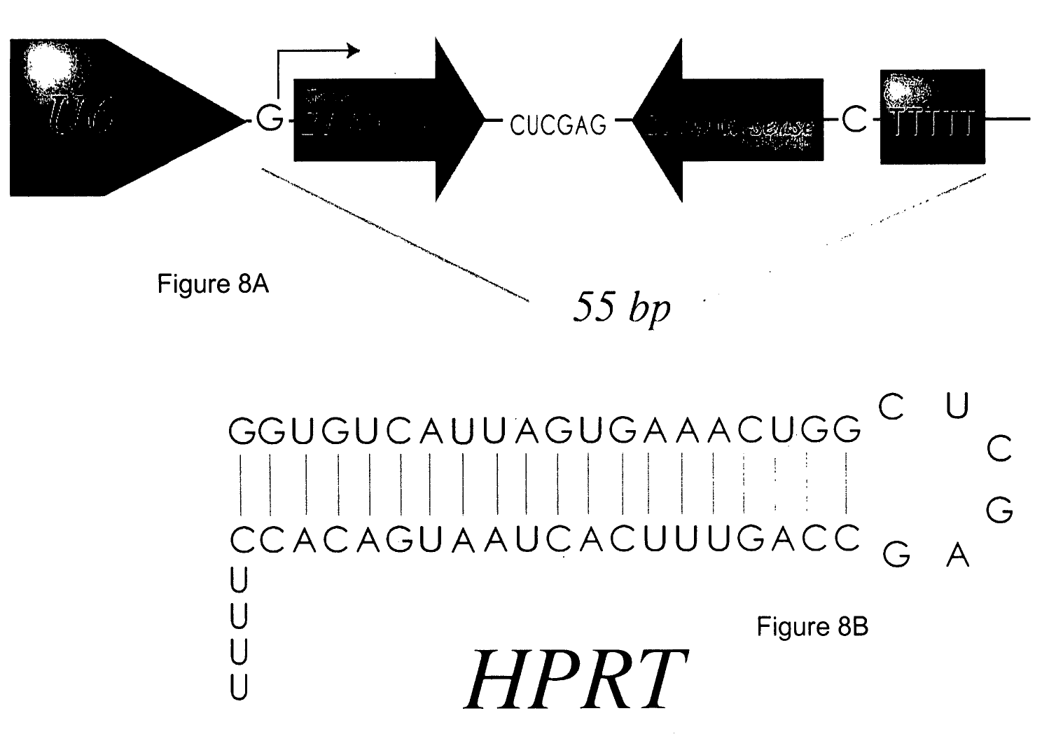

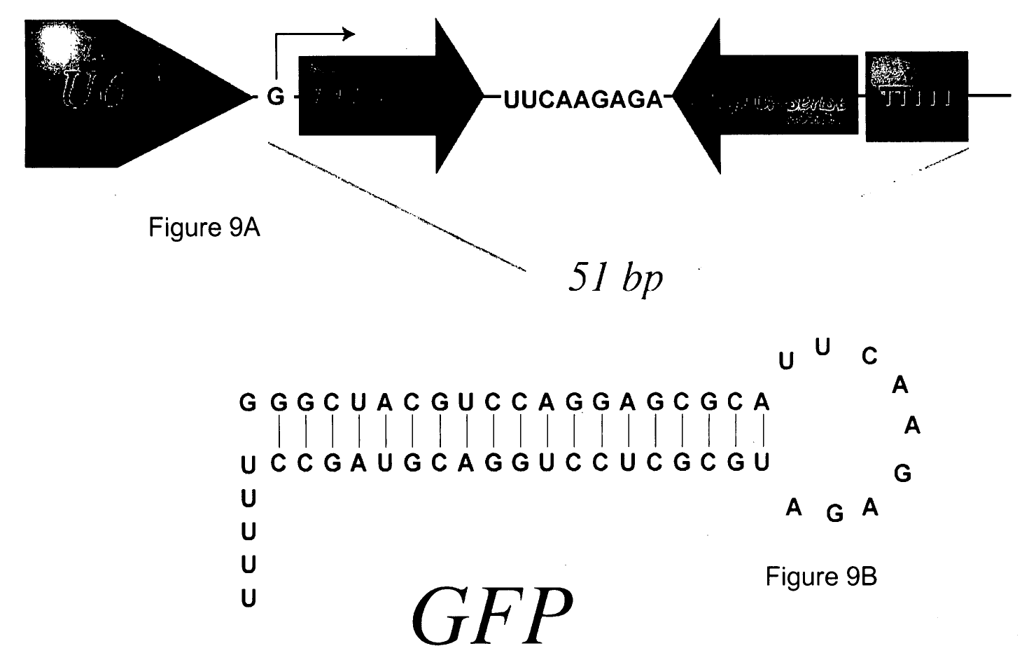

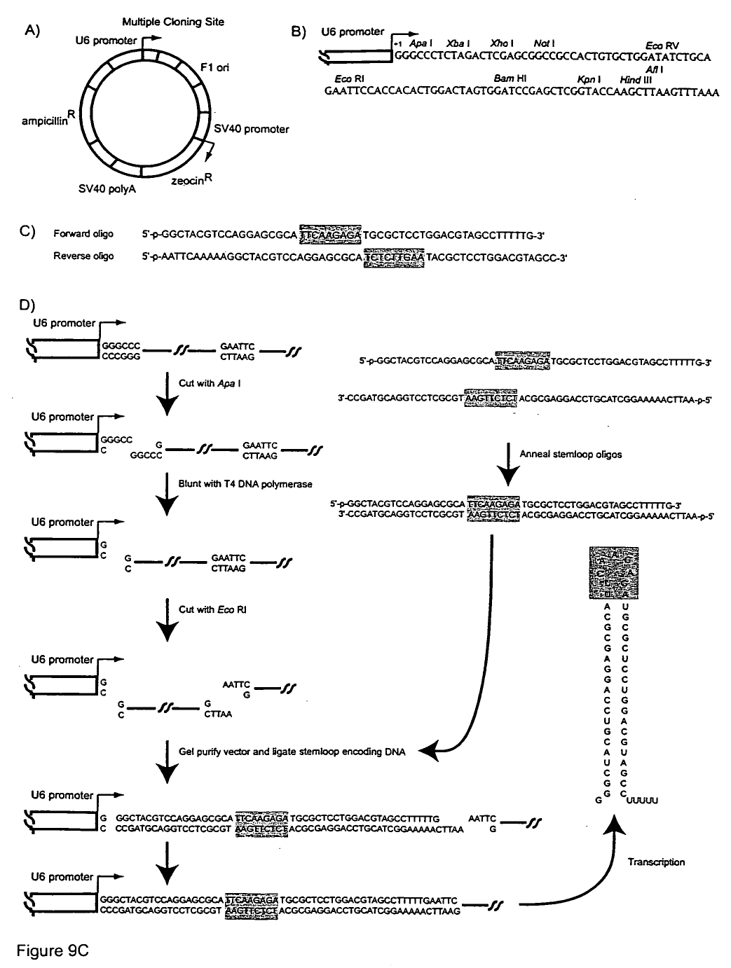

The general characteristics of vectors that can be used to provide expression of RNAi-inducing agents such as shRNAs or siRNAs were described above. In particular embodiments of the invention a vector such as that depicted in FIG. 7 is employed. This vector includes a U6 promoter that drives transcription of an operably linked nucleic acid that serves as a template for transcription of a shRNA. The shRNA contains 21 bp sense and antisense strands connected by a 6 nt loop. An overhang comprising several U residues (corresponding to the T residues shown in the figure) is present at the 3′ end of the shRNA. An shRNA such as this, targeted to HPRT, is shown in FIG. 8B. In general, the shRNA can be targeted against any marker by appropriate selection of the sense and antisense sequences. An shRNA targeted to GFP is shown in FIG. 9B. The vector can be introduced into cells expressing the marker, which results in RNAi-mediated inhibition of expression. It will be appreciated that constructs such as those described above that provide templates for transcription of other RNAi-inducing agents such as siRNAs or precursors such as microRNA precursor hairpins may be similarly created and introduced into cells.

In addition to individual vectors that provide templates for transcription of RNAi-inducing agents targeted to a single selectable or detectable marker such as HPRT, destabilized enhanced GFP, etc., the invention provides a nucleic acid comprising a template for transcription of a first RNAi-inducing agent targeted to a first marker and operably linked to a promoter and a template for transcription of a second RNAi-inducing agent targeted to a second marker and operably linked to a promoter. The invention further provides vectors that comprise this nucleic acid. The promoters can be the same or different but should be suitable for expression of RNAi-inducing agents (e.g., PolI or Pol III promoters such as the U6 promoter, H1 promoter, tRNA promoter, appropriate Pol II promoters).

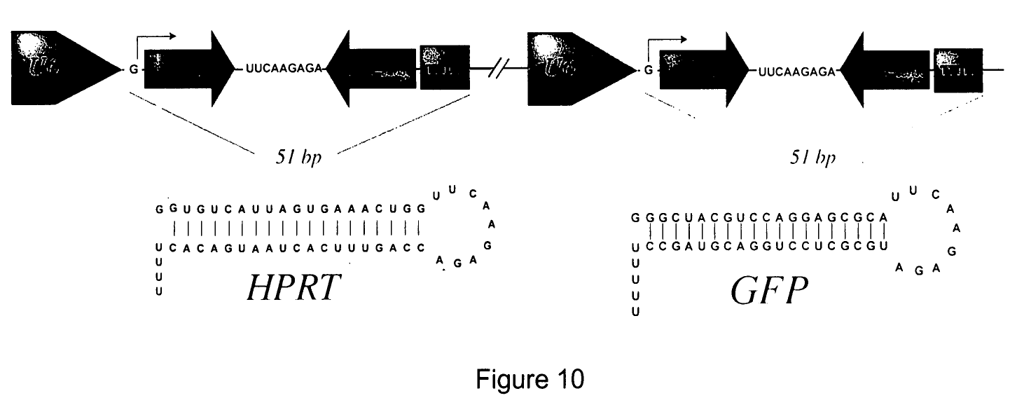

In general the first and second markers will be different although they could be the same. Various combinations are possible. For example, the first marker may be selectable and the second marker detectable, or vice versa, or both markers may be selectable or detectable. FIG. 10 shows an example of a cassette that can be inserted into a vector such as that shown in FIG. 7 in place of the insert shown (i.e., the U6 promoter and downstream shRNA sequences). Two shRNAs that are transcribed from this insert are also depicted. Alternately, a vector such as that of FIG. 7 can be modified to include one or more additional transcription units for transcription of an RNAi-inducing agent. Other methods of creating the vector can also be used.

The invention provides a nucleic acid comprising (i) a template for transcription of a first RNAi-inducing agent targeted to a selectable or detectable marker and operably linked to a first promoter and (ii) a second promoter and a site for insertion of a template for transcription of an RNAi-inducing agent located downstream of the promoter, so that the template will be operably linked to the promoter once inserted. The invention further provides vectors that comprise this nucleic acid. The promoters can be the same or different but should be suitable for expression of RNAi-inducing agents. In certain embodiments of the invention the nucleic acid also comprises a portion that codes for the selectable or detectable marker. In certain embodiments of the invention the marker is encoded by an endogenous gene, e.g., HPRT, TK, or DHFR. In preferred embodiments of the invention the endogenous gene is naturally expressed by the cell. However, in certain embodiments of this and other aspects of the invention, a nucleic acid comprising a coding sequence encoding the marker, operably linked to appropriate regulatory sequences, is introduced into the cell to augment expression of the endogenous gene. The nucleic acid may be stably integrated into the cellular genome.

Such vectors have a number of uses. For example, as mentioned above, it is more difficult to transfect, infect, or otherwise introduce and express nucleic acids in certain cells or cell types than others. In addition, there may be variability in the degree to which certain cells or cell types will silence a target gene even in response to introduction of the same RNAi-inducing agent into the cells. Furthermore, certain genes appear to be more difficult to silence than others, e.g., such genes may only be effectively silenced in a small number of cells out of a population that receives the same RNAi-inducing agent. These factors may make it difficult to use RNAi and to interpret results of attempting to silence a gene using RNAi. For example, if only a small proportion of cells effectively silence a gene, then the phenotype that results from silencing may be obscured by the non-silenced phenotype. Therefore, for this and other reasons there exists a need for methods to identify and/or select cells in which effective silencing is taking place.

The inventors have recognized that by co-delivering to a population of cells a template for transcription of an RNAi-inducing agent targeted to a gene of interest together with a template for transcription of an RNAi-inducing agent targeted to a transcript that encodes a marker such as a selectable or detectable marker that is expressed by the cell and then identifying cells that no longer express the marker, it is possible to identify cells in which RNAi is active, and thereby identify cells in which there is an increased likelihood that the gene of interest is silenced. In preferred embodiments of the invention the templates for transcription of the two RNAi-inducing agents are inserted into a single vector in which they are operably linked to suitable promoters for expression of RNAi-inducing agents. For example, the promoters can be RNA polymerase III promoters such as U6 or H1 or RNA polymerase I promoters.

The selectable or detectable marker can be any selectable or detectable marker that is expressed by the cell and for which it is possible to identify cells that have decreased expression of the marker relative to cells that do not express an RNAi-inducing agent targeted to a transcript that encodes the marker. The selectable marker can be an endogenous gene, e.g., HPRT or TK. If the marker is not an endogenous gene, then either the cells should already express the marker (e.g., because of previous introduction of a nucleic acid that encodes the marker into the cells) or a nucleic acid that encodes the marker, operably linked to a promoter, should be included in the construct that delivers one or both of the RNAi-inducing agents to the cell.

In certain embodiments of the invention a template for transcription of an RNAi-inducing agent targeted to a gene of interest is inserted into a vector such as those described immediately above, e.g., a vector that comprises a template for transcription of an RNAi-inducing agent operably linked to a first promoter and further comprises a second promoter and a site for insertion of a template for transcription of an RNAi-inducing agent downstream of the second promoter so that a nucleic acid inserted into the site will be expressed. Such vectors can be provided in the form of a kit, so that a user can conveniently insert a template for transcription of an RNAi-inducing agent targeted to a gene of choice into the site for insertion of such a template, introduce the resulting construct into a population of cells of choice and identify cells in which RNAi is active by applying appropriate selective conditions or detection methods. The kit may contain any of the components described below in the section discussing kits.

Thus the invention provides a method of identifying a cell in which a gene of interest is silenced by RNAi comprising steps of: (i) introducing into cells of a cell population a nucleic acid comprising (a) a template for transcription of a first RNAi-inducing agent targeted to a selectable or detectable marker and operably linked to a first promoter and (b) a second promoter and a site for insertion of a template for transcription of an RNAi-inducing agent located downstream of the promoter, so that the template will be operably linked to the promoter once inserted into a population of cells, wherein the nucleic acid further comprises a template for transcription of an RNAi-inducing agent targeted to the gene of interest; and (ii) identifying a cell in which RNAi is active by selecting or detecting cells that do not express the selectable or detectable marker, thereby identifying a cell in which the gene of interest is silenced by RNAi. If the marker is a selectable marker the step of identifying generally comprises exposing the cells to selective conditions that select against cells that express the selectable marker. In certain embodiments of the invention the marker is an endogenous gene. In certain embodiments of the invention the marker is selected from the group consisting of HPRT or TK. If the marker is a selectable marker the step of identifying may comprise exposing the cells to a compound that is processed by the selectable marker to yield a toxic compound. For example, if the marker is HPRT, then a suitable vector may contain a template for transcription of an shRNA targeted to HPRT operably linked to a U6 promoter. The vector would further contain a second promoter, e.g., a U6 promoter, upstream of a site into which a user could insert a template for transcription of an RNAi-inducing agent targeted to a gene of interest. The resulting construct is introduced into a population of cells. Cells in which RNAi is active will silence HPRT while cells in which RNAi is not active will express HPRT. Cells in which RNAi is active can be selected by placing them in medium containing 8-AZ or 6-TG. Cells that have silenced HPRT because their RNAi pathway is active will have a significant growth advantage under these selective conditions. It is thereby possible to select cells in which there is an increased likelihood that the gene of interest is silenced.

Kits

The invention provides a variety of kits that can be employed, e.g., to practice the inventive methods described below for identification of genes involved in an RNAi pathway or compounds that modulate RNAi, or for other purposes. In certain embodiments of the invention, the kits contain one or more of the inventive cell lines described above and one or more additional components that are used to practice the methods and/or as controls. Some of these components are described in the following section. In addition to one or more inventive cell lines, certain of the kits include one or more of the following (i) an RNAi-inducing agent that targets an mRNA that encodes the marker; (ii) an RNAi-inducing agent that does not target an mRNA that encodes the marker; (iii) a compound (e.g., a small molecule) that inhibits RNAi; (iv) a compound (e.g., a small molecule) that activates RNAi; (v) a genetic element that inhibits RNAi; (vi) a genetic element that activates RNAi; (vii) an RNAi-inducing agent that targets an mRNA that encodes Dicer; (viii) one or more compounds for addition to tissue culture medium to impose a selective condition on the mammalian cell line included in the kit; (ix) a cell line that comprises a nucleic acid that encodes the same marker as the collection of cell lines but does not comprise a template for transcription of an RNAi-inducing agent that reduces expression of the marker; (x) a vector comprising a U6, H1, or tRNA promoter and a site downstream of the promoter for insertion of a template for transcription of an RNAi-inducing agent; (xi) a transfection reagent; and (xii) instructions for use.

The invention also provides kits containing vectors such as pSHARP, i.e., vectors that contain a promoter for transcription of an RNAi-inducing agent such as an shRNA in a vector backbone containing an antibiotic resistance marker that provides resistance to an antibiotic such as zeocin, hygromycin, G418, or puromycin. The vector also contains a site for insertion of a template for transcription of an RNAi-inducing agent located downstream of the promoter, so that the template will be operably linked to the promoter once inserted. The promoter may be any promoter suitable for expression of an RNAi-inducing agent. In certain embodiments of the invention the promoter is U6, H1, or a tRNA promoter. The kit may contain a plurality of such vectors, each having a different antibiotic resistance marker.

In addition, certain kits contain one or more sets of vectors such as (i) a vector comprising a U6, H1, or tRNA promoter and a site downstream of the promoter for insertion of a template for transcription of an RNAi-inducing agent; and (ii) the vector of (i) into which a template for transcription of an RNAi-inducing agent that is targeted to a selectable marker (e.g., HPRT) or a detectable marker (e.g., destabilized eGFP) is inserted. The selectable or marker may be any of the selectable or detectable markers discussed herein. The vectors may contain the same antibiotic resistance marker. A user can insert a template for transcription of an RNAi-inducing agent targeted to a transcript of choice into the site for insertion in vector (i) and introduce the resulting vector into cells. The companion vector (ii) containing a template for transcription of an RNAi-inducing agent targeted to a selectable or detectable marker may be used as a control to confirm that effective silencing is occurring. Additional kit components such as cell lines of the invention, etc., can also be included.

### V. Methods for Identifying Genes Involved in RNAi Pathways

Genetic Screens Using Chemical or Insertional Mutagenesis

A discussed above, the inventors have recognized that cells and cell lines that express a selectable or detectable marker (e.g., existing cells that express an endogenous gene such as HPRT or cells that are engineered so that they express an appropriate marker) can be modified to render them useful for identification of genes involved in RNAi and/or compounds that modulate RNAi. According to certain aspects of the invention, cells that comprise a nucleic acid that encodes a selectable marker are modified so that they stably express an RNAi-inducing agent that silences expression of the marker. A variety of such cells are described above. Thus when RNAi is active in the cell expression of the marker is inhibited, while if RNAi is inactivated, e.g., as a result of a loss of function mutation in a gene involved in an RNAi pathway, or as a result of exposure to a compound that inhibits RNAi), the marker is expressed. Expression of the marker is thus used as a basis to identify cells having alterations in RNAi pathways.

Thus the invention provides a method for identifying a gene involved in an RNAi pathway comprising steps of: (a) providing a population of mammalian cells, members of which comprise a nucleic acid that encodes a detectable or selectable marker and further comprise one or more templates for transcription of an RNAi-inducing agent that reduces expression of the detectable or selectable marker; (b) mutagenizing the population of cells; and (c) identifying cells that display decreased or increased expression of the detectable or selectable marker relative to the starting population, thereby identifying cells that have a mutation in a gene involved in an RNAi pathway. Mutagenesis can be performed using a variety of methods that are known in the art, e.g., using chemical mutagens such as ethyl methylsulfonate (EMS) or ethyl nitrosourea (ENU), both of which have been titrated to generate defined numbers of mutations in CHO cells (Klungland, A., K. Laake, E. Hoff, and E. Seeberg. 1995. Spectrum of mutations induced by methyl and ethyl methanesulfonate at the hprt locus of normal and tag expressing Chinese hamster fibroblasts. Carcinogenesis 16:1281-1285; Mohn, G. R., and A. A. van Zeeland. 1985. Quantitative comparative mutagenesis in bacteria, mammalian cells, and animal-mediated assays. A convenient way of estimating genotoxic activity in vivo? Mutat Res 150:159-175), by using radiation, etc. Cells having increased or decreased expression of the marker can be identified using various techniques depending upon the particular marker employed. Suitable techniques are discussed above. For example, where the marker is HPRT, cells having decreased expression of the marker can be identified by selecting them in a compound such as 8-AZ that is metabolized to a cytotoxic compound by HPRT. Such cells are candidates for having a gain of function mutation in a gene involved in the RNAi pathway by which the RNAi-inducing agent silences expression of the marker. The gain of function mutation potentiates RNAi, thus decreasing expression of the marker and allowing cell growth under conditions that would otherwise result in cell death. Cells having increased expression of the marker can be identified by selecting them in HAT medium. Such cells are candidates for having a loss of function mutation in a gene involved in the RNAi pathway by which the RNAi-inducing agent silences expression of the marker. The loss of function mutation reduces the efficacy of RNAi, thus increasing expression of the marker and allowing cell growth under conditions that would otherwise result in cell death.

If the marker is a detectable marker, cells having increased or decreased expression of the marker can be identified using a suitable screening method, e.g., using FACS for fluorescent markers such as GFP. Single cell cloning is then performed to generate clonal cell lines having the mutation. In certain embodiments of the invention genetic selection and screening are both performed. For example, RNAi-cells (i.e., cells lacking RNAi or having reduced RNAi efficacy) may be identified either by their growth in HAT (because they express HPRT) or by GFP expression. Using these cells, GFP expression is used to independently verify that disruption of the RNAi pathway was the cause of the growth phenotypes. Selections and screens to identify cells having gain of function mutations in an RNAi pathway gene can be similarly identified. Identified cells may be further maintained in culture to generate a cell line.

Genetic complementation can be used to further identify mammalian RNAi genes. Heterokaryon hybridomas can be generated by polyethylene glycol (PEG) treatment of RNAi-mutant cells and complementation groups defined by the combinations of RNAi-cells that when fused form heterokaryon cells that are RNAi+. This procedure has been used for a variety of purposes, e.g., to define complementation groups in the definition of the low-density lipoprotein receptor pathway in CHO cells (Hobbie, L, et al., J Biol Chem., 269(33):20958-70, 1994). Complementation groups can be defined by the ability of the heterokaryon cells to survive in media containing 8-AZ and their lack of growth when cultured in media containing HAT. In addition, complementation group assignments can be confirmed by reconstitution of GFP silencing phenotype by FACS analysis.

Insertional mutagenesis provides to randomly disrupt mammalian genes offers another approach to the generation of RNA-cells and may be preferred because of the ease with which the mutated genes can be identified. Insertional mutagenesis is generally performed by infecting cells with a retrovirus that integrates into the genome. If the virus integrates into a transcription unit, transcription will frequently be disrupted, resulting in loss of gene expression. Thus in general the strategy involves (i) isolating large populations of cells in which proviruses have integrated extensively in the genome; (ii) selecting cell clones for phenotypes that result when gene function is lost as a result of integration (e.g., an RNAi-phenotype); (iii) identifying and characterizing specific genes disrupted by the integrated provirus (Goff, S. P., Methods Enzymol., 151: 489-502, 1987). Retrovirus infection of pools of mammalian cells followed by shotgun sequencing of the integration sites has been described (29). In addition, improved retroviral vectors such as “gene trap” vectors are available (Chang, W., et al., Virology, 193: 737-747, 1993, and references therein). Such vectors typically include a selectable marker so that selective conditions select for cells in which integration into a transcriptionally active region of the genome has occurred, thus increasing the likelihood that disruption of a functional gene will occur.

A variety of approaches can be used to identify the genes responsible for the RNAi lacking (RNAi minus) phenotypes, depending upon the method of generating RNAi minus cells. If insertional mutagenesis leads to RNAi minus cells, then PCR with universal primers to the insertional cassettes can be used to amplify regions of the disrupted genes. Alternatively, if chemical mutagenesis leads to the generation of RNAi minus cells, genetic complementation of RNAi minus phenotypes can be achieved by infecting RNAi minus mutant cells with retroviruses harboring cDNA libraries of expressed mammalian genes from mouse and/or human sources (commercially available, e.g., from Statagene, Clontech, etc.). Retrovirus infected cells can then be grown in media containing 8-AZ to verify reversal of the RNAi minus phenotype. Only cells with intact RNAi, actively silencing HPRT expression, should grow. The reversed phenotype can be confirmed independently by GFP fluorescence by FACS analysis of cells expressing GFP-hairpin RNA and actively silencing GFP expression. Genes responsible for phenotypic reversion to wild type can be identified by PCR amplification, e.g., using universal primers to retroviral sequences. Thus, the identity of the genes responsible for the silencing deficient phenotype can be discovered. Confirmation of the genes involved in the RNAi pathway can be achieved by ectopic expression of these genes in the RNAi-(RNA negative) cells. Proof of their involvement may be confirmed by their ability to confer an RNAi positive phenotype to the RNAi minus cells. Biochemical complementation studies and other biochemical approaches can also be used.

The invention further provides a method of identifying a gene involved in a miRNA translational repression pathway comprising steps of: (a) providing a population of mammalian cells members of which comprise (i) a nucleic acid that is integrated into the genome of the cell and provides a template for transcription of an mRNA transcript that encodes a detectable or selectable marker, wherein the mRNA transcript comprises one or more binding sites for an endogenous miRNA or an miRNA-like RNA; and (ii) an endogenous miRNA or miRNA-like RNA that is expressed by the cell and represses translation of the mRNA that encodes a detectable or selectable marker; (b) mutagenizing the population of cells; and (c) identifying cells that display decreased or increased expression of the detectable or selectable marker relative to the starting population and do not display an alteration in mRNA transcript level sufficient to account for the increased or decreased expression of the marker, thereby identifying cells that have a mutation in a gene involved in a miRNA translational repression pathway. In certain preferred embodiments of the invention the nucleic acid is a reporter construct that encodes luciferase as a marker. Luciferase expression may conveniently be measured by assaying luciferase activity. The miRNA or miRNA-like RNA partially or completely silences luciferase expression. A mutation that reduces or eliminates function of a gene that is involved in an miRNA translational repression pathway reduces the efficacy of translational represson, which is detectable as an increase in expresson of the marker (e.g., luciferase). A mutation that increases the function of a gene that is involved in an miRNA translational repression pathway increases the efficacy of translational repression, which is detectable as a decrease in expression of the marker (in the event that expression was not fully repressed originally). In order to confirm that the mutation does indeed affect an miRNA pathway, the level of the transcript can be measured (e.g., using a ribonuclease protection assay, RT-PCR, Northern blot, etc.). If the level of the transcript remains approximately the same or any change in transcript levels is insufficient to account for the observed change in expression of the marker, then it can be concluded that the alteration in expression arose as a result of mutation in a gene involved in an miRNA translational repression pathway. If desired, the gene can then be cloned using standard techniques or otherwise identified. In certain embodiments of the invention the cell also expresses an siRNA or siRNA precursor such as an shRNA, targeted to the transcript that encodes the marker. Alternately, the cell may express a different marker and an siRNA or shRNA targeted to that marker. Such cells can be used to selectively identify genes involved in either siRNA or miRNA pathways, or both. In addition to, or instead of, making direct measurement of transcript levels to confirm that the mutation affects an miRNA pathway, a comparison with a control cell that is otherwise identical but in which the mRNA transcript encoding the marker lacks sufficient or appropriate miRNA binding sites to mediate translational repression can be performed. For example, the transcript may contain miRNA binding site(s) for an miRNA that is not present in the cell.

Genetic Element Screens

The invention provides additional methods that may be used to identify genes involved in an RNAi pathway. Certain of these methods make use of libraries, e.g., cDNA libraries, comprising genetic elements (GE). According to the inventive methods a library of genetic elements is introduced into a population of mammalian cells having a functioning RNAi pathway, and cells in which RNAi is inhibited or activated by the element are identified. The identity of the genetic element is determined as described below. In certain embodiments of the invention the genetic element is a genetic suppressor element (GSE) in that it inhibits or suppresses a gene to which it corresponds. The gene suppressed by the genetic suppressor element can then typically be identified and/or cloned using methods well known in the art. Since suppression of the gene results in reduced or activated RNAi, the expression product of the gene is likely to function in RNAi either directly (e.g., as a component of RISC) or indirectly (e.g., by regulating the expression and/or activity of a molecule that functions directly in RNAi).

GSE screens have been used to identify genes involved in a number of cellular pathways such as apoptosis and tumor suppression. Similar approaches, referred to as “death trap”, “technical knockout”, or “MaRX” have also been used for such purposes. GSE screens and related methods for gene identification are described in Gudkov, A. V., et al., Proc. Natl. Acad. Sci., 91: 3744-3748; Hannon, G., Science, 283: 1129, 1998; Deiss, L. P. and Kimchi, A., Science, 252: 117, 1991; Holzmayer, T. A., et al., Nucl. Acids Res., 20: 711, 1992; Gudkov, A. V. and Roninson, I., Methods Mol. Biol., 69: 221, 1997; Kimchi, A., et al., Science, 285: 299a, 1999 and references therein. In general, such screens involve introduction of a cDNA library into cells and identification and recovery of GSEs based on a phenotypic screen or selection. Recovered GSEs can be introduced into new cells and additional rounds of phenotypic selection or screening (e.g., using different selectable or detectable markers) can be employed. GSEs that transduce the phenotype of interest are scored as positives.

In general, genetic suppressor elements are nucleic acids, e.g., cDNAs, that encode protein fragments that act as inhibitors of protein function and/or provide templates for transcription of antisense RNA molecules that bind to a complementary mRNA and inhibit gene expression by a mechanism believed to be distinct from RNAi. Thus in general a genetic suppressor element corresponds to a particular gene (and its expression product(s)), i.e., the gene that encodes the protein fragment (for the first type of GSE) or the gene from which the mRNA complementary to the antisense molecule is transcribed (for the second type of GSE). Expression of the first type of GSE, which typically encodes a short protein fragment, e.g., a single functional domain, may inhibit protein function in any of a variety of ways. For example, the protein fragment may compete for binding to a substrate or downstream target (squelching). It is noted that although a genetic suppressor element generally inhibits the corresponding gene, the result of inhibition may be activation or potentiation of a biological pathway or phenomenon, e.g., a biological pathway or phenomenon that is normally suppressed by an expression product of the gene.

In addition, the isolation of active proteins or protein fragments using a library of cDNAs that encode proteins or protein fragments and/or provide templates for transcription of antisense RNA molecules that bind to a complementary mRNA is encompassed by certain embodiments of the invention, in which case the term “genetic element” rather than “genetic suppressor element” more accurately reflects the activity of the cDNA. Alternately, the protein or protein fragment may act as an alternative substrate for a negative regulator of the protein to which the genetic element corresponds. In this case the genetic element does not function to inhibit a corresponding gene but rather results in enhancement of its function. Thus use of the term “genetic element” or “genetic suppressor element” is not intended to limit the invention to elements that are inhibitory or to limit the range of genes that can be identified using the screens described herein.

One method for identifying cells containing a genetic element that inhibits or activates an RNAi pathway comprises steps of: (a) providing a first population of mammalian cells, members of which comprise a nucleic acid that encodes a first detectable or selectable marker and express an RNAi-inducing agent that reduces expression of the detectable or selectable marker; (b) introducing a library into the population of cells, wherein the library comprises a plurality of genetic elements; and (c) identifying cells that display increased or decreased expression of the detectable or selectable marker relative to the starting population, thereby identifying cells that contain a genetic element that inhibits or activates an RNAi pathway, respectively. The invention further provides a method for identifying cells containing a genetic element that inhibits an RNAi pathway comprises steps of: (a) providing a first population of mammalian cells, members of which comprise a nucleic acid that encodes a first detectable or selectable marker and express an RNAi-inducing agent that reduces expression of the detectable or selectable marker; (b) introducing a library into the population of cells, wherein the library comprises a plurality of genetic elements; and (c) identifying cells that display increased expression of the detectable or selectable marker relative to the starting population, thereby identifying cells that contain a genetic element that inhibits an RNAi pathway.

The invention also provides a method for identifying cells containing a genetic element that activates an RNAi pathway comprises steps of: (a) providing a first population of mammalian cells, members of which comprise a nucleic acid that encodes a first detectable or selectable marker and express an RNAi-inducing agent that reduces expression of the detectable or selectable marker; (b) introducing a library into the population of cells, wherein the library comprises a plurality of genetic elements; and (c) identifying cells that display decreased expression of the detectable or selectable marker relative to the starting population, thereby identifying cells that contain a genetic element that activates an RNAi pathway. In certain embodiments of the inventive methods the GE library comprises sequences from a first species and is introduced into cells from a second species. For example, the GE library may comprise human sequences and may be introduced into CHO cells (hamster). When PCR is performed on genomic DNA isolated from colonies of cells that are identified as positive using a screen or selection, the use of a primer specific for genes of the first species may reduce the likelihood of amplification of an endogenous cellular gene. Alternately, the library may be introduced into cells of the species from which the elements in the library were obtained.

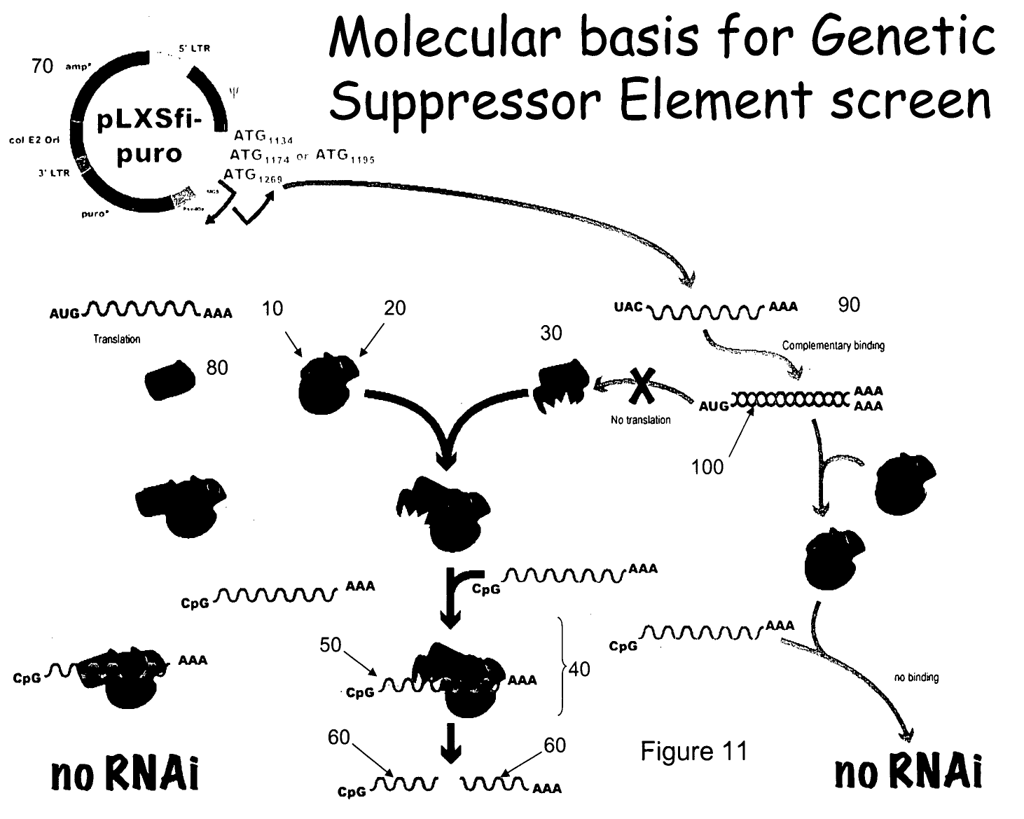

FIG. 11 presents an overview of the molecular basis for a screen to identify a GE that inhibits RNAi. In this screen the GE inhibits a corresponding gene and may therefore be referred to as a GSE. The middle portion of the figure shows a portion of the RNAi pathway, in which various components 10, 20, and 30 involved in RNAi associate with each other to form an active silencing complex 40 (e.g., components of the RNA induced silencing complex (RISC) associate to form RISC), which cleaves a target transcript 50 to generate RNA cleavage products 60. The upper left portion of the figure shows a retroviral vector, pLXSfipuro, 70 having cDNA inserts inserted in all three reading frames and in both forward and reverse orientation with respect to the promoter. Transcription in the forward orientation followed by translation (left side of figure) yields protein fragments, among them protein fragment 80 that binds to components 10 and/or 20, thereby preventing binding of component 30 and preventing formation of an active silencing complex. Thus RNAi is inhibited in cells that receive a GE that encodes protein fragment 80.

The right side of FIG. 11 shows an alternate mechanism by which GEs can inhibit RNAi. Transcription of a cDNA inserted in the reverse orientation yields an antisense RNA molecule 90 complementary to a transcript 100 that encodes component 30. Binding of the antisense molecule to transcript 100 prevents translation. In the absence of component 30 formation of an active silencing complex cannot take place, thus RNAi is prevented.

Recovery and identification of the GSE that encodes protein fragment 80 or the GSE that provides a template for transcription of antisense RNA 90 is used to identify the gene whose expression is inhibited by the GSE. For example, the sequence of the GSE can be used to search a genome sequence, e.g., the human genome sequence, to find an identical or highly homologous sequence that is part of the gene. Databases containing the human genome sequence and other genome sequences are publicly available and known to one of ordinary skill in the art. Alternately, the GSE can be used to probe a cDNA library to isolate a longer cDNA, preferably a full length cDNA, that corresponds to the gene. PCR using a primer corresponding to a sequence in the GSE or in a longer cDNA clone, together with a universal primer such as oligodT or a mixture random primers, can be used to isolate additional sequence if necessary. Similar methods may be used to perform screens using GEs and to determine their identity.

GEs may also encode full length proteins, though in certain embodiments of the invention it is preferred to employ a cDNA library in which the cDNAs are size selected, e.g., selected to be less than approximately 2 kb, less than approximately 1 kb, between 200 and 1000 bp, between 200 and 500 bp, etc. In addition, in certain embodiments of the invention it is desirable to employ a normalized cDNA library. In general, the cDNA fragments are inserted into a vector suitable for introduction into mammalian cells. Preferably the cDNAs are inserted at a site in which they are operably linked to a strong promoter active in mammalian cells, e.g., in all three reading frames.

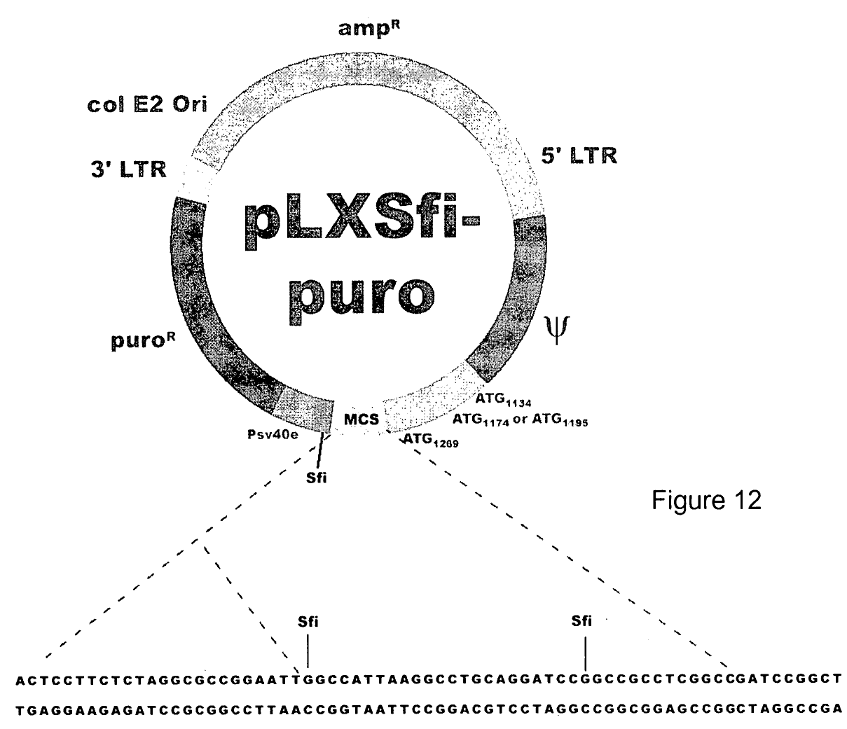

A variety of vectors can be used for construction of a GE library. The vector can be a DNA plasmid that can be introduced into cells using standard transfection methods. The vector may, but need not be, a retroviral vector that can be introduced into cells by transfection or used to produce retroviruses that are used to infect cells, thereby introducing the library. Methods for production of infectious retroviruses are well known in the art and a variety of commercial cDNA libraries employing retroviral vector systems are available, e.g., the ViraPort™ XR plasmid cDNA library from Stratagene. Production of virus generally involves introducing a retroviral vector comprising certain viral long terminal repeats (LTRs) and a packaging signal into cells (packaging cells) that provide necessary viral components such as Gag-Pol and Env proteins in trans. Alternatively, the retroviral vector can be cotransfected together with plasmids that encode these proteins. Infectious virus buds from the cell and is released into the culture medium, from which it can be recovered. FIG. 12 depicts one suitable vector (pLXSfipuro) that can be used for construction of a GE library and for production of infectious retrovirus. In certain embodiments of the invention an episomal vector capable of replicating as an episome in mammalian cells is used, e.g., an EBV-based vector. Such vectors may have a number of advantages, e.g., they are easily rescued from cells and reduce the background of unrelated mutations resulting from random integration into the genome as may occur with retroviruses.

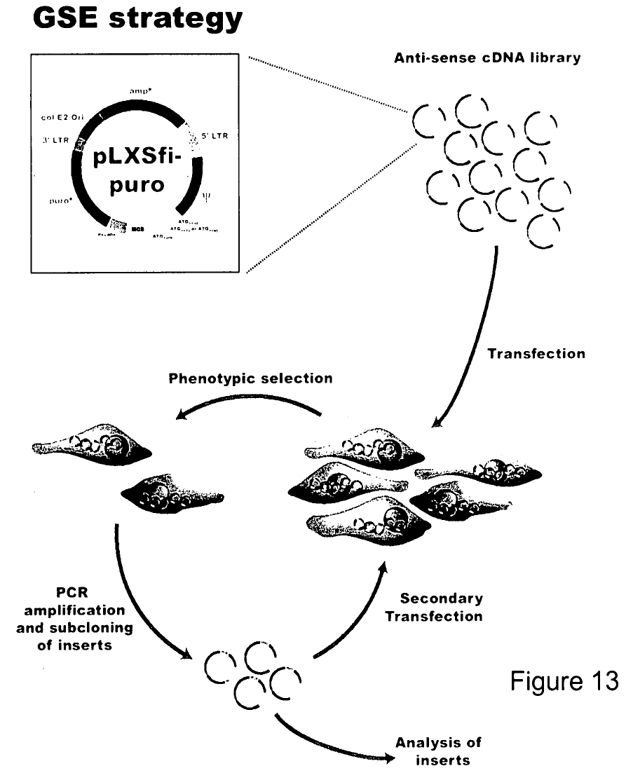

FIGS. 13-18 illustrate a number of variations of the GE screening and selection strategy that can be used in accordance with the invention. FIG. 13 shows a method in which the GE library comprises cDNA sequences inserted in reverse orientation relative to the promoter in a retroviral vector so that transcription in a recipient cell results in production of RNA complementary to mRNA transcripts. The vector harboring the antisense library is then transfected into cells such as those described above, e.g., cells that comprise a nucleic acid that encodes a selectable or detectable marker and that express an RNAi-inducing agent that silences expression of the marker. Transfectants are typically selected using puromycin (not shown) and are subjected to selection or detection. For example, if the marker is HPRT, cells are cultured in HAT medium. Only cells in which HPRT is expressed, e.g., cells in which the RNAi pathway that would otherwise silence HPRT expression is suppressed by the antisense RNA present in the cell should survive the selection. The inserts are then amplified by PCR using vector-specific primers located on either side of the insert. Inserts may then be further analyzed, e.g., by sequencing, to identify the gene that is inhibited by the insert. The sequence can be used to search a database, e.g., the human genome sequence database to identify the corresponding gene. Inserts can also be used to probe a cDNA or genomic library to identify longer cDNA or genomic clones. Inserts can be cloned into the original vector (or any other suitable vector) and transfected into a new population of recipient cells which is then subjected to another round of selection. Performing multiple rounds of selection enriches for inserts that reproducibly disrupt silencing.

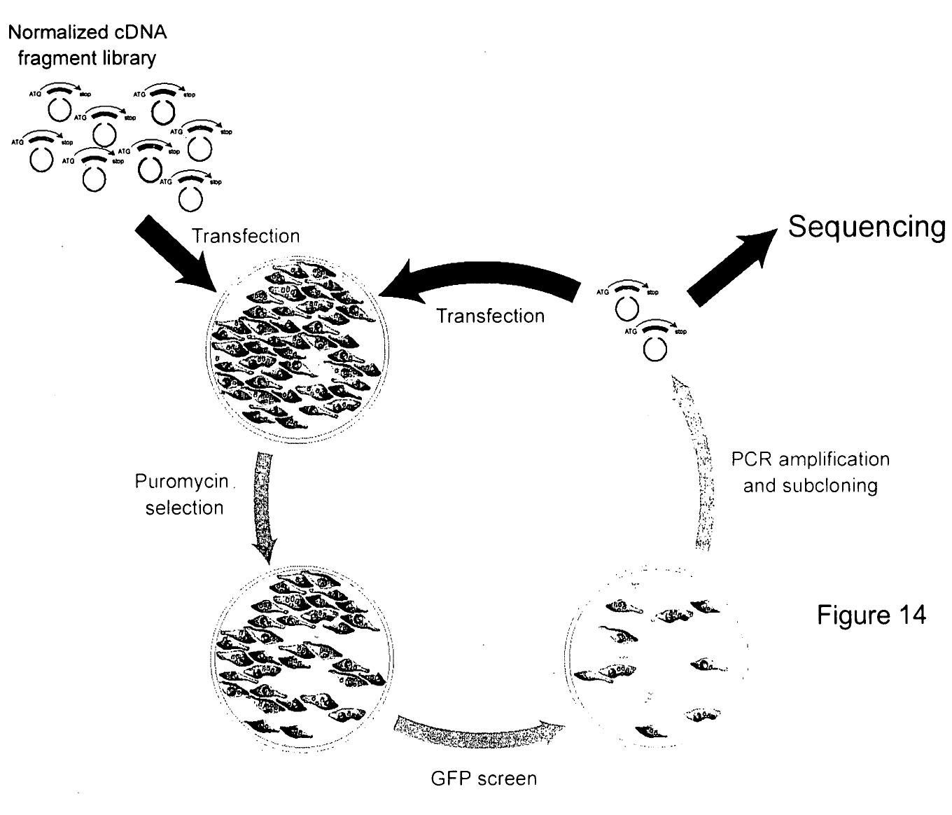

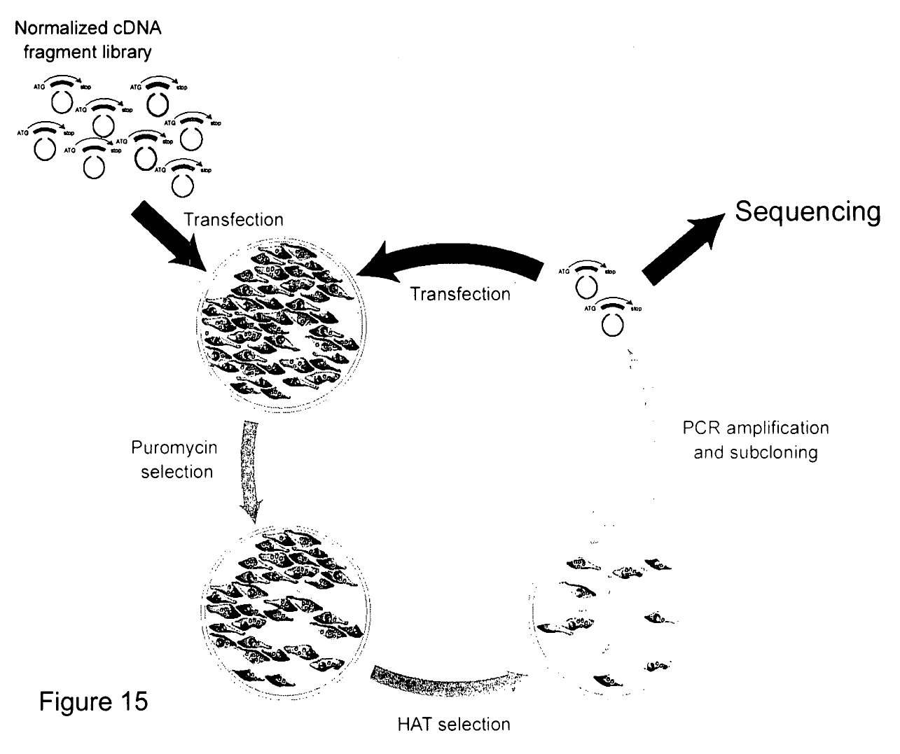

FIG. 14 presents another example of an inventive method for identifying a GE that inhibits a gene involved in an RNAi pathway. A library comprising normalized cDNA fragments inserted into a retroviral vector is transfected into recipient cells such as those described above that contain a nucleic acid encoding GFP and express an RNAi-inducing agent such as an shRNA targeted to GFP that silences GFP expression. Transfectants selected using puromycin are subjected to screening to identify cells that express GFP, e.g., cells in which RNAi is inhibited by the GE. Genomic DNA is isolated from positive colonies and PCR is performed using vector-specific primers. The PCR products are sequenced and/or cloned into the original vector (or another suitable vector). The clones are then introduced into new recipient cells, which are subjected to further rounds of screening to enrich for GEs that reproducibly disrupt RNAi. FIG. 15 shows a similar method except that the cells contain a nucleic acid that encodes HPRT (e.g., the endogenous HPRT gene) and express an RNAi-inducing agent that inhibits HPRT expression, e.g., an shRNA targeted to HPRT. Transfectants are subjected to selection in HAT medium to identify cells in which HPRT is expressed, e.g., cells in which RNAi is disrupted. Additional steps are performed as described for FIG. 14.

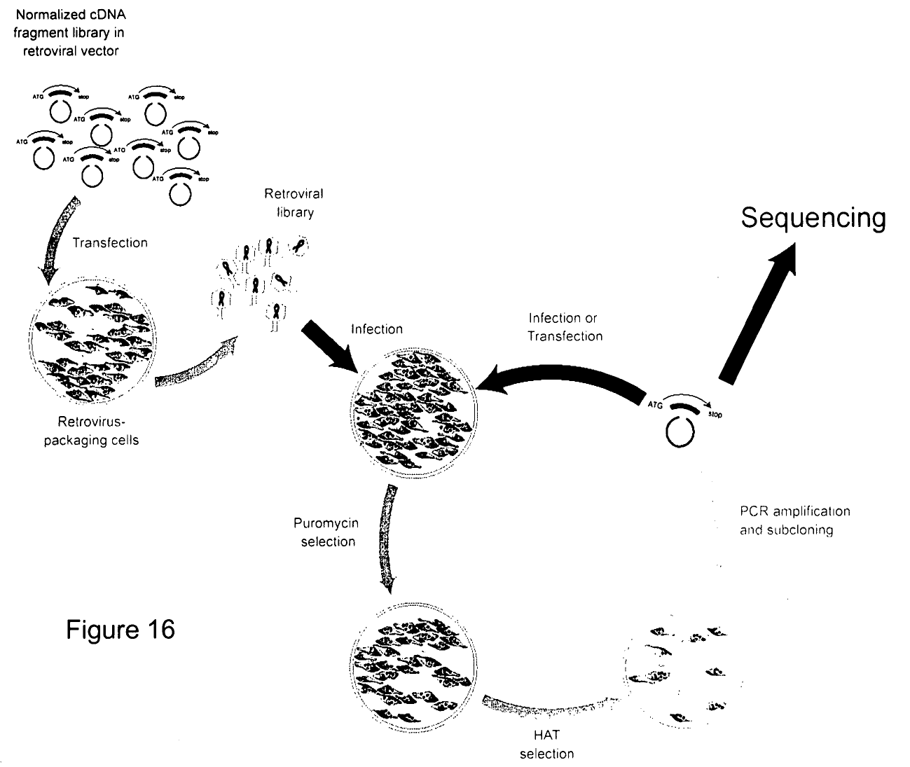

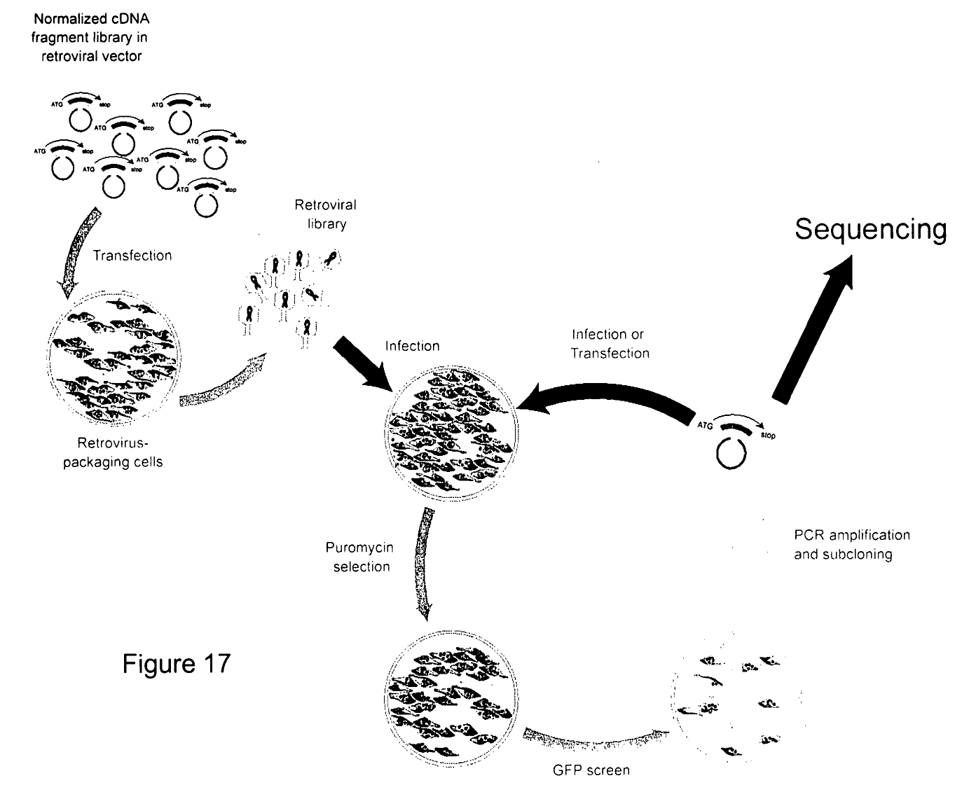

FIGS. 16 and 17 show methods similar to those of FIGS. 14 and 15 except that the retroviral vectors are used to produce infectious retrovirus, e.g., by transfecting them into a packaging cell line that provides additional viral components such as Gag-Pol and Env proteins in trans or by cotranfecting the retroviral vectors together with additional constructs that code for these components. Retroviruses are harvested and used to introduce the library into recipient cells, which are then selected using puromycin and subjected to screening for GFP expression or selection in HAT medium for HPRT expression. Additional steps are performed as described above.

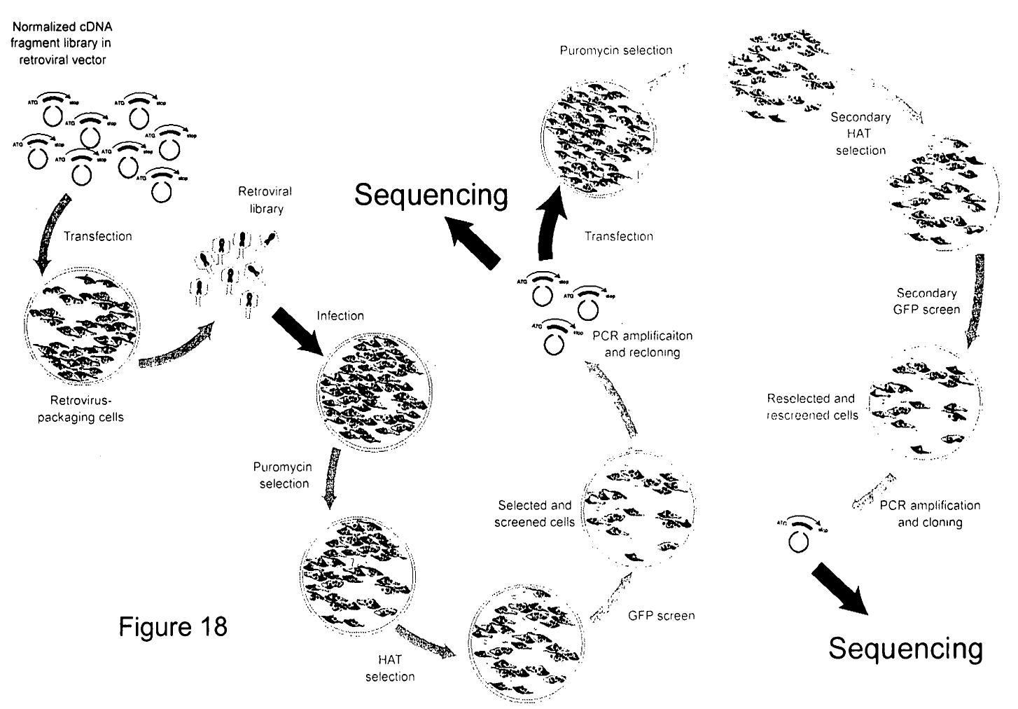

FIG. 18 shows a variation in which the cells contain nucleic acids that encode two markers, in this case a selectable marker (HPRT) and a detectable marker (GFP), and express RNAi-inducing agents targeted to both markers. A vector such as that described above that provides templates for transcription of two RNAi-inducing agents can be used to create the cell line. Retroviruses are produced as described above and used to infect the cells. Infected cells are subjected to a selection for HPRT expression using HAT medium. Selected cells are then screened for GFP expression. Genomic DNA is prepared from cells that pass both the HAT selection and the GFP screen. By performing both a selection and a secondary screen, using two different markers, the likelihood of recovering GEs that actually disrupt RNAi is increased. Different combinations of screens and selections can be used and/or additional rounds of selection or screening using the same or different markers can be performed. PCR amplification is used to recover the GEs, which are then subcloned and/or sequenced as described above.

One of ordinary skill in the art will readily perceive that numerous variations on and extension of the above methods will also be effective. In general, any cells containing nucleic acids encoding appropriate marker(s) and RNAi-inducing agents can be used, though the cells of the invention described above may be preferred. Other methods of recovering the GE may be used. For example, Cre recombinase has been used to recover retroviral inserts from selected cells (Sun, P. et al., Science, 282: 2270, 1998; Li, L. and Cohen, S. N., Cell, 85:319, 1996).

It is also noted that the antisense molecules or protein fragments corresponding to the genetic suppressor elements identified as inhibitors of RNAi can be delivered to cells to inhibit or activate RNAi. These antisense molecules or protein fragments can be delivered exogenously. Alternatively, a vector that provides a template for transcription of an inhibitory antisense molecule or that encodes an inhibitory protein fragment can be introduced into cells and the inhibitory antisense molecule or protein fragment can then be expressed intracellularly to inhibit or activate RNAi in the cell.

It is further noted that rather than screening for cells in which RNAi is inhibited, the above methods can be used to identify cells in which RNAi is activated or potentiated. For example cells containing a nucleic acid that encodes GFP and that express an RNAi-inducing agent that partially silences GFP can be transfected with a GE library and cells in which silencing of GFP is enhanced can be isolated. The genes inhibited by the GEs present in such cells are likely to be negative regulators of RNAi (i.e., they function to reduce RNAi) or inhibitors of RNAi. For example, such a gene may be a kinase that phosphorylates and thereby inhibits a component of the RNAi machinery, or a phosphatase that dephosphorylates and thereby inhibits a component of the RNAi pathway. Selection in medium containing 8-AZ or 6-TG can be used similarly to identify genes that are negative regulators or inhibitors of RNAi.

The invention also provides a method for identifying cells containing a genetic element that inhibits or activates a miRNA translational repression pathway comprising steps of: (a) providing a first population of mammalian cells members of which comprise (i) a nucleic acid that is integrated into the genome of the cell and provides a template for transcription of an mRNA transcript that encodes a detectable or selectable marker, wherein the mRNA transcript comprises one or more binding sites for an endogenous miRNA or an miRNA-like RNA; and (ii) an endogenous miRNA or miRNA-like RNA that is expressed by the cell and represses translation of the mRNA that encodes a detectable or selectable marker; (b) introducing a library into the population of cells, wherein the library comprises a plurality of genetic elements; (c) identifying cells that display increased or decreased expression of the detectable or selectable marker relative to the starting population and do not display an alteration in mRNA transcript level sufficient to account for the increased or decreased expression of the marker, thereby identifying cells that contain a genetic element that inhibits or activates an miRNA translational repression pathway, respectively. In certain preferred embodiments of the invention the nucleic acid is a reporter construct that encodes luciferase as a marker. Luciferase expression may conveniently be measured by assaying luciferase activity. The miRNA or miRNA-like RNA partially or completely silences luciferase expression. A GE that reduces or eliminates function of a gene that is involved in an miRNA translational represson pathway reduces the efficacy of translational represson, which is detectable as an increase in expresson of the marker (e.g., luciferase). A GE that increases the function of a gene that is involved in an miRNA translational repression pathway increases the efficacy of translational repression, which is detectable as a decrease in expression of the marker (in the event that expression was not fully repressed originally). In order to confirm that the GE does indeed affect an miRNA translational repression pathway, the level of the transcript that encodes the marker can be measured (e.g., using a ribonuclease protection assay, RT-PCR, Northern blot, etc.). If the level of the transcript remains approximately the same or any change in transcript levels is insufficient to account for the observed change in expression of the marker, then it can be concluded that the alteration in expression arose as a result of the GE. If desired, the GE can be isolated as described above. In certain embodiments of the invention the cell also expresses an siRNA or siRNA precursor such as an shRNA, targeted to the transcript that encodes the marker. Such cells can be used to selectively identify GEs involved in either an siRNA RNAi pathway or an miRNA translational repression pathway, or both.

In addition to, or instead of, making direct measurement of transcript levels to confirm that the GE affects an miRNA translational repression pathway, a comparison with a control cell that is otherwise identical but in which the mRNA transcript encoding the marker lacks sufficient or appropriate miRNA binding sites to mediate translational repression can be performed. For example, the transcript may contain miRNA binding site(s) for an miRNA that is not present in the cell. If the GE also affects silencing in the control cell line, then it is unlikely that the compound specifically affects only an miRNA pathway and more likely that, for example, it affects transcript levels or some aspect of translation other than miRNA-mediated translational repression in addition to, or instead of affecting miRNA-mediated translational repression.

### VI. Methods for Identifying Compounds that Modulate RNAi

The invention provides a number of methods for identifying chemical compounds, e.g., small molecules, that inhibit or activate RNAi. One such method for identifying a compound that inhibits or activates RNA interference comprises steps of: (a) providing a population of mammalian cells that comprise a nucleic acid that encodes a detectable or selectable marker and express an RNAi-inducing agent that reduces expression of the detectable or selectable marker by RNA interference; (b) contacting the cells with a compound; (c) identifying the compound as an inhibitor of RNAi if cells exhibit enhanced expression of the detectable or selectable marker after being contacted with the compound relative to cells not contacted with the compound or identifying the compound as an activator of RNAi if cells exhibit reduced expression of the detectable or selectable marker after being contacted with the compound relative to cells not contacted with the compound.

For example, the cells may comprise a nucleic acid that encodes a detectable marker such as GFP and express an RNAi-inducing agent such as a shRNA targeted to GFP, so that GFP expression is low or absent. Following exposure to a test compound, cells are sorted by FACS to identify cells that have increased GFP expression, e.g., relative to other cells in the population or relative to control cells that have not been exposed to compound. Increased GFP expression serves as an indication that RNAi is inhibited. Thus the compound is identified as a candidate inhibitor of RNAi. It is generally preferred to use cells that have a sharp GFP peak and display a strong RNAi phenotype (i.e., GFP expression is reduced to a low level). Alternately, cells that comprise a nucleic acid that encodes HPRT and express an RNAi-inducing agent targeted to HPRT that inhibits HPRT expression are exposed to a test compound and cultured in HAT medium. The cells may be pretreated with the compound prior to placing them in HAT medium and/or after placing them in HAT medium. The compound may be present continuously during culturing in HAT medium. Cells that survive in HAT medium are likely to exhibit expression of HPRT, indicating that RNAi is inhibited. The compound is thus identified as a candidate inhibitor of RNAi.

Similar methods can be used to identify compounds that are activators of RNAi. For example, cells that comprise a nucleic acid encoding a detectable marker such as GFP and express an RNAi-inducing agent that inhibits expression of the marker are contacted with a test compound. Following exposure to the test compound, cells are sorted by FACS to identify cells that have decreased GFP expression, e.g., relative to other cells in the population or relative to control cells that have not been exposed to compound. Decreased GFP expression serves as an indication that RNAi is activated. Thus the compound is identified as a candidate activator of RNAi. It is generally preferred to use cells that have a sharp GFP peak and display a relatively weak or intermediate RNAi phenotype (i.e., GFP expression is reduced by RNAi but is not fully inhibited), so that further reduction in GFP expression will result in a detectable decrease in fluorescence. Alternately, cells that comprise a nucleic acid that encodes HPRT and express an RNAi-inducing agent targeted to HPRT that inhibits HPRT expression are exposed to a test compound and cultured in medium containing 8-AZ or 6-TG which, as discussed above, are converted into cytotoxic compounds by HPRT. The cells may be pretreated with the compound prior to placing them in selective medium and/or after placing them in the selective medium. The compound may be present continuously during culturing in selective medium. Cells that survive or have a growth advantage in medium containing 8-AZ or 6-TG are likely to exhibit decreased expression of HPRT, indicating that RNAi is activated. The compound is thus identified as a candidate activator of RNAi.

It will be appreciated that numerous variations on the above methods are possible. For example, different populations of cells contacted with a compound may be subjected to a screening step and a selection step. Cells can be exposed to a range of different compound concentrations and can be pretreated with compound for different amounts of time prior to screening or selection. Different markers can be used. For example, cells can be engineered to express an RNAi-inducing agent targeted to DHFR, so that the de novo purine synthesis pathway is inhibited. Cells are then contacted with a test compound and those that are able to grow in standard tissue culture medium lacking hypoxanthine and thymidine are selected. These cells are likely to have a disabled RNAi pathway. The compound is thus identified as a candidate inhibitor of RNAi. Compounds that cause (i) derepression of the de novo purine synthesis pathway (i.e., derepression of DHFR expression); (ii) derepression of GFP expression; and (iii) derepression of HPRT expression are highly likely to be inhibitors of RNAi since they will have demonstrated effects consistent with inhibition of RNAi-inducing agents that inhibit expression of three independent targets.

The invention also provides a method for identifying a compound that inhibits or activates an miRNA translational repression pathway comprising steps of: (a) providing a population of mammalian cells members of which comprise (i) a nucleic acid that is integrated into the genome of the cell and provides a template for transcription of an mRNA transcript that encodes a detectable or selectable marker, wherein the mRNA transcript comprises one or more binding sites for an endogenous miRNA or an miRNA-like RNA; and (ii) an endogenous miRNA or miRNA-like RNA that is expressed by the cell and represses translation of the mRNA that encodes a detectable or selectable marker; (b) contacting the cells with a compound; and (c) identifying the compound as an inhibitor of a miRNA translational repression pathway if cells exhibit enhanced expression of the detectable or selectable marker after being contacted with the compound relative to cells not contacted with the compound and do not display enhanced mRNA transcript levels sufficient to account for the enhanced expression of the marker, or identifying the compound as an activator of an miRNA translational repression pathway if cells exhibit reduced expression of the detectable or selectable marker after being contacted with the compound relative to cells not contacted with the compound and do not display decreased mRNA transcript levels sufficient to account for the reduced expression of the marker. In certain preferred embodiments of the invention the nucleic acid is a reporter construct that encodes luciferase as a marker. Luciferase expression may conveniently be measured by assaying luciferase activity. The miRNA or miRNA-like RNA partially or completely silences luciferase expression. A compound that reduces or eliminates function of a gene that is involved in an miRNA translational repression pathway reduces the efficacy of translational represson, which is detectable as an increase in expresson of the marker (e.g., luciferase). A compound that increases the function of a gene that is involved in an miRNA translational repression pathway increases the efficacy of translational repression, which is detectable as a decrease in expression of the marker (in the event that expression was not fully repressed originally). In order to confirm that the compound does indeed affect an miRNA translational repression pathway, the level of the transcript that encodes the marker can be measured (e.g., using a ribonuclease protection assay, RT-PCR, Northern blot, etc.). If the level of the transcript remains approximately the same or any change in transcript levels is insufficient to account for the observed change in expression of the marker, then it can be concluded that the alteration in expression arose as a result of exposure to the compound. In certain embodiments of the invention the cell also expresses an siRNA or siRNA precursor such as an shRNA, targeted to the transcript that encodes the marker. Such cells can be used to selectively identify compounds involved in either siRNA RNAi pathways or miRNA translational repression pathways, or both.

In addition to, or instead of, making direct measurement of transcript levels to confirm that the compound affects an miRNA pathway, a comparison with a control cell that is otherwise identical but in which the mRNA transcript encoding the marker lacks sufficient or appropriate miRNA binding sites to mediate translational repression can be performed. For example, the transcript may contain miRNA binding site(s) for an miRNA that is not present in the cell. If the compound also affects silencing in the control cell line, then it is unlikely that the compound specifically affects an miRNA pathway and more likely that, for example, it affects transcript levels or some aspect of translation other than miRNA-mediated translational repression in addition to, or instead of affecting miRNA-mediated translational repression.

In certain preferred embodiments of the invention screening and/or selection are performed using a high throughput approach, using robotics and automation techniques that are well known in the art to test multiple members of a compound library. FIG. 19 represents a schematic overview of a typical screening or selection process in which cells are dispensed into an assay plate whose wells contain test compounds. See Example 7 for further details. Numerous variations are possible. For example, multiple compounds can be placed in each well. The compounds in a well that scores positively can then be individually tested. In general, any compound library can be used. Numerous libraries are available commercially or have been reported in the literature, e.g., from Chembridge (described at the web site having URL iccb.med.harvard.edu/screening/compound_libraries/chembridge.html, or Comgenex. Without limiting the invention, suitable libraries include libraries of synthetic compounds, combinatorial libraries, natural product libraries, etc.

In certain embodiments of the invention an annotated compound library (ACL) is used such as that described in Root, D. E., et al., Chemistry & Biology, 10:881-892, 2003. This library contains 2036 small organic molecules representing a large-scale collection of compounds with diverse, experimentally confirmed biological mechanisms and effects. Considerable information is available about each of the compounds, facilitating the development of hypotheses regarding mechanism of action (see Example 7). Following identification of positives, compounds known to have similar biological activities and/or mechanisms of action, or known to be structurally related, can be tested. In addition, substituents at key positions can be altered. For example, substituents known to be required for a particular mechanism may be removed. If the resulting compound fails to have the same effect in an inventive screen or selection as the parent compound, this would suggest that the known mechanism of the parent compound is relevant to the mechanism by which it affects RNAi. Systematically modifying substituents at key positions will also likely yield compounds with enhanced RNAi-activating or inhibiting activity.

In another aspect of the invention, a compound that acts as an inhibitor or activator of RNAi can be used as reagents to purify the targets of such inhibitor or activator. For example, in accordance with the inventive method, cells are treated with a compound that is an inhibitor or activator of RNAi. The compound is then isolated from the cells using any suitable means, e.g., using an antibody that binds to the compound. The isolation may be performed under mild conditions, e.g., nondenaturing conditions, to enhance the likelihood that any molecule bound to the compound will remain bound during the isolation procedures and will co-purify with the compound. Alternatively, the compound may be modified to incorporate a cross-linkable group such as an SH group, photo-crosslinkable group, etc., prior to addition of the compound to the cells. The cells or cell lysate may then be exposed to conditions appropriate to result in cross-linking prior to isolation of the compound, thereby resulting in isolation of any moieties cross-linked to the compound, e.g., protein or RNA molecules with which the compound associates within the cell. Such protein or RNA molecules are candidates components of the cellular RNAi machinery.

In another embodiment of the invention, in order to facilitate isolation of the compound, the compound is modified to include a moiety (e.g., biotin, Myc tag, hemagglutinin (HA) tag, 6×-His tag, glutathioine-S-transferase (GST), FLAG tag, etc.), that binds to a binding agent (such as streptavidin in the case of biotin, nickel in the case of His, glutathione in the case of GST), or an antibody in the case of Myc, HA, GST, and FLAG) prior to addition of the compound to the cells. The compound and any associated molecules may then be conveniently isolated using the binding agent according to methods that are well known in the art. In order to minimize the likelihood that the moiety will interfere with the ability of the compound to inhibit or activate RNAi, a variety of derivatives may be synthesized and tested in order to identify those derivatives that retain the desired RNAi-inhibiting or RNAi-activating activity.

In certain embodiments of the invention an agent such as an aptamer that specifically binds to the compound is used to isolate the compound. For example, in vitro selection has been used to isolate nucleic acid sequences (aptamers) that bind small molecules with a high degree of affinity and specificity. Aptamers that bind to any of a wide variety of molecules may be generated using standard techniques such as “systematic evolution of ligands by exponential enrichment” (SELEX) that are well known in the art See, e.g., Werstuck, G. and Green, M., Science, 282: 296-298, 1998; Landweber, L, Trends in Ecology and Evolution, 14(9): 353-358, 1999; Herman, T. and Patel, D., Science, 287: 820-825, 2000, and references in the foregoing. See also Clark S L and Remcho V T, Electrophoresis, 23(9):1335-40, 2002, describing use of aptamers for a variety of applications including as affinity reagents.

The target of a compound that inhibits or activates RNAi is a candidate component of an RNAi pathway. Following purification of the candidate component the corresponding gene (i.e., a gene that provides a template for transcription of an RNA component such as a protein-associated small RNA, or a gene that encodes a polypeptide component such as a constituent of the RISC) may be identified using any of a number of approaches that are well known in the art. For example, an RNA component may be reverse transcribed and sequenced or used as a probe, e.g., to isolate genomic sequence complementary to the RNA. A polypeptide component may be sequenced (e.g., using N-terminal microsequencing), and the sequence then used to design degenerate oligonucleotides that are then used as probes to identify a cDNA or genomic sequence that encodes the polypeptide. According to another approach, N-terminal sequence data are used to prepare anti-peptide antibodies that bind to the component, e.g., in order to facilitate purification of larger quantities of the component. Such antibodies may also be used to deplete the component to which they bind from cell extracts, which can then be used to perform biochemical complementation studies.

### VII. Computer-Readable Media and Databases

In order to utilize, process, share, present, display, transmit, receive, and/or store information obtained using any of the inventive methods described herein, it may be desirable to enter such information into a computer system and/or store it on a computer-readable medium such as a hard disk, compact disk, zip disk, floppy disk, flash memory, etc. In general, the information may be stored in computer-readable format (e.g., digital format) on any type of computer-readable medium. The invention thus provides a computer-readable medium containing computer-readable information indicating that a gene, mutation, genetic element, or compound affects an RNAi pathway, wherein the gene, mutation, genetic element, or compound was identified as affecting an RNAi pathway by any of the methods described herein. Further information may be provided, e.g., the extent to which the RNAi pathway is affected, whether an siRNA or miRNA pathway, or both, is affected, etc. Experimental results may be included. The information may be present in a database. Suitable information includes, but is not limited to, (i) the name and/or accession number and/or sequence and/or restriction map of a gene or genetic element (either a wild type or mutant form), the common name, chemical name, and/or structure of a compound; (iii) an indication of the extent to which a mutation, genetic element, or compound affects expression of a transcript, etc. While computer-readable media may be the most convenient format for the processing, storing, sharing, presentation, etc., of information obtained using the methods of the invention, the invention also encompasses any tangible medium, i.e., any physical medium having substance or material existence, generally perceptible to the touch, on or in which such information is present.

### VIII. Pharmaceutical Formulations

As mentioned above, RNAi is widely used in mammalian cells grown in culture and in mammalian organisms, e.g., for functional studies of genes. In addition, animal studies have indicated that RNAi-inducing agents are likely to have therapeutic applications. Thus compounds that inhibit or activate RNAi are useful in mammalian tissue culture systems and in animal studies and also for therapeutic purposes. The invention therefore provides pharmaceutical compositions comprising chemical inhibitors or activators of RNAi identified according to the inventive methods described above.

Inventive compositions (e.g., compounds that activate or inhibit an RNAi pathway) may be formulated for delivery by any available route including, but not limited to parenteral (e.g., intravenous), intradermal, subcutaneous, oral, nasal, bronchial, opthalmic, transdermal (topical), transmucosal, rectal, and vaginal routes. Preferred routes of delivery include parenteral, transmucosal, nasal, bronchial, and oral. Inventive pharmaceutical compositions may also include an RNAi-inducing agent in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Solutions or suspensions used for parenteral (e.g., intravenous), intramuscular, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. Preferred pharmaceutical formulations are stable under the conditions of manufacture and storage and must be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Preferably solutions for injection are free of endotoxin. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the inventive compounds are preferably delivered in the form of an aerosol spray from a pressured container or dispenser which contains a suitable propellant, e.g., a gas such as carbon dioxide, or a nebulizer. Liquid aerosols, dry powders, etc., can be used.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

The compounds or compositions may be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

A therapeutically effective amount of a pharmaceutical composition typically ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The pharmaceutical composition can be administered at various intervals and over different periods of time as required, e.g., multiple times per day, daily, every other day, once a week for between about 1 to 10 weeks, between 2 to 8 weeks, between about 3 to 7 weeks, about 4, 5, or 6 weeks, etc. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject can include a single treatment or, in many cases, can include a series of treatments.

Exemplary doses include milligram or microgram amounts of the inventive compound per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.) For local administration (e.g., intranasal), doses much smaller than these may be used. It is furthermore understood that appropriate doses of a compound depend upon its potency and may optionally be tailored to the particular recipient, for example, through administration of increasing doses until a preselected desired response is achieved. It is understood that the specific dose level for any particular animal subject may depend upon a variety of factors including the activity of the specific compound employed, the age, body weight, general health, gender, and diet of the subject, the time of administration, the route of administration, the rate of excretion, any drug combination, and the degree of expression or activity to be modulated.

As mentioned above, the present invention includes the use of inventive compositions for treatment of nonhuman animals including, but not limited to, horses, swine, and birds. Accordingly, doses and methods of administration may be selected in accordance with known principles of veterinary pharmacology and medicine. Guidance may be found, for example, in Adams, R. (ed.), Veterinary Pharmacology and Therapeutics, 8^{th} edition, Iowa State University Press; ISBN: 0813817439; 2001.

Inventive pharmaceutical compositions can be included in a container, pack, or dispenser together with instructions for administration.

### EXAMPLES

### Example 1

Design and construction of RNAi vectors for identification of RNAi pathway genes and chemical modulators.

Standard molecular biology techniques as described, for example, in Sambrook, et al., referenced above, were used for all cloning steps in this and other examples. As a first step toward generating vectors that mediate stable RNAi when introduced into mammalian cells, the U6 promoter was cloned into a pCDNA3.1-zeocin backbone (Invitrogen) to generate pSHARP-zeocin. The U6 promoter was similarly cloned into vector backbones that contained the hygromycin or puramycin markers, to create a versatile family of pSHARP vectors (pSHARP-zeocin, pSHARP-hygromycin, pSHARP-puramycin), into which templates for transcription of an RNAi-inducing agent of interest can be cloned. The resulting vectors were otherwise identical to pSHARP-zeocin. FIG. 7 shows the pSHARP-zeocin vector with a generic insert for transcription of an shRNA. For purposes of description members of the pSHARP family will be referred to generically as pSHARP without specifically identifying the antiobiotic resistance gene, unless otherwise indicated.

To construct a vector for silencing GFP expression (pSHARP-shGFP), an inverted repeat sequence (stem-loop, sl) with a stem sequence whose antisense strand is perfectly complementary to a portion of the GFP mRNA, followed by a five thymidine repeat sequence to serve as a strong Pol-III termination signal (Gunnery, S., et al., J. Mol. Biol., 286: 745-757, 1999), was cloned into an Eco RI site downstream of the U6 promoter in pSHARP-hygromycin. In order to do so, the vector was digested with Apa I and the restriction site was blunted using T4 DNA polymerase to chew back the 3′ overhang and then filled in (in the presence of excess dNTPs) to generate a blunt end on the vector while maintaining the +1 G residue. DNA oligomers were annealed and cloned into vector that had first been digested with Eco RI. The resulting construct was transformed into E. coli, and colonies were screened by DNA sequencing to ensure that the DNA oligomers had been properly ligated into the vector. The following DNA oligomers were used:

| | |
|---|---|
| GFP1 (forward): | |
| 5′-P-GGCTACGTCCAGGAGCGCACCCTCGAGGGTG | (SEQ ID NO: 1) |
| cgctcctggacgtagcctttttg-3′ | |
| GFP1 (reverse): | |
| 5′-P-AATTCAAAAAGGCTACGTCCAGGGCGACCCT | (SEQ ID NO: 2) |
| CGAGGGTGCGCTCCTGGACGGAGCC-3′ | |

In addition to the stem, the RNA structure that results from transcription from the U6 promoter includes a 9 nucleotide spacer with sequence 5′-UUCAAGAGA-3′ (Brummelkamp, T. R., et al., Science, 296: 550-553, 2002) connecting the nucleotides that form the base pair at one end of the stem. FIG. 9A shows the vector insert containing the U6 promoter, the regions that hybridize to form the stem whose antisense strand is complementary to the GFP mRNA in the transcribed RNA, and the 9 nucleotide spacer

FIG. 9B shows an exemplary predicted RNA structure resulting from transcription of an shRNA targeted to GFP from the U6 promoter. It is noted that the first and last nucleotides in the spacer are complementary and may hybridize to each other rather than forming part of the loop. FIG. 9C shows a schematic outline of a method for inserting a template for transcription of an shRNA targeted to GFP into a vector containing a U6 promoter, in this case pSHARP-zeocin. The shRNA contains a 6 nt loop sequence, which is shaded in the figure.

To construct a vector for silencing HPRT expression (pSHARP-shHPRT), an inverted repeat sequence with a stem sequence whose antisense strand is perfectly complementary to a portion of the human HPRT gene, followed by the five thymidine repeat sequence was cloned into pSHARP as described for the creation of pSHARP-shGFP using DNA oligomers that include the stem sequences separated by a 6 nucleotide spacer.

In addition to the stem sequences, the hairpin structure that results from transcription from the U6 promoter includes a 6 nucleotide spacer with sequence 5′-CUCGAG-3′ (Sui, G., et al., Proc. Natl. Acad. Sci., 99:5515-5520, 2002) connecting the nucleotides that form the base pair at one end of the stem. FIG. 8A shows the vector insert containing the U6 promoter, the stem-loop sequences that hybridize to form the stem in the transcribed RNA, and the 6 nucleotide spacer between these two portions. FIG. 8B shows an exemplary predicted hairpin structure resulting from transcription of an shRNA targeted to HPRT from the U6 promoter. It is noted that the first and sixth and the second and fifth nucleotides in the spacer (loop) are complementary and may hybridize rather than forming part of the loop, although based on steric considerations it is unlikely that such hybridization actually occurs.

A dual vector for silencing both GFP and HPRT expression was created using standard cloning techniques. The vector contains a cassette that provides promoters and templates for transcription of shRNAs targeted to GFP and HPRT (shown in FIG. 10), in the same backbone as that used to create pSHARP.

### Example 2

Production and Testing of Clonal Cell Lines Expressing GFP and an shRNA That Silences GFP Expression

Experimental Procedures

Cell culture and single cell cloning. HeLa cells were grown in Dulbecco Modified Eagle medium (DMEM) plus 10% heat-inactivated fetal calf serum (FCS) containing penicillin and streptomycin at 37° C. with 5% CO₂. Chinese hamster ovary (CHOk1) cells were grown in F-12 medium plus 10% heat-inactivated FCS containing penicillin and streptomycin at 37° C. with 5% CO₂.

To generate stable cell lines expressing GFP, HeLa and CHOk1 cells were transfected with pdlEGFP-N1 (Clontech) using standard techniques. This vector encodes an enhanced version of the GFP protein with the PEST domain of ornithine decarboxylase at the carboxy terminus, resulting in a fusion protein with enhanced fluorescence compared with the original GFP gene and a shortened half-life of approximately 1 hour, Transfectants were selected with 500 μg/ml G418 and single cell cloned to generate stable GFP-expressing cell lines, HeLa-GFP and CHO-k1-GFP, that exhibited high fluorescence with a sharp peak. Single cell cloning was performed by subjecting transfectants to single cell sorting using a fluorescence activated cell sorter (FACS) according to standard techniques. Cells that displayed high levels of fluorescence were retained and were dispensed into 96 well trays at approximately 1 cell per tray. Wells containing colonies were identified and the colonies were expanded. Clones displaying a sharp and strong fluorescence peak were identified by FACS. HeLa-GFP and CHO-k1-GFP cells were transfected with pSHARP-shGFP or pSHARP-shHPRT and selected with hygromycin and zeocin respectively.

To obtain clonal CHOk1 cell lines expressing GFP and an RNA hairpin targeted to GFP, parental CHO-k1-GFP cells expressing GFP were transfected with pSHARP-shGFP, selected using the appropriate antibiotic for 2-3 weeks and subjected to single cell sorting using a fluorescence activated cell sorter (FACS) according to standard techniques. Cells that displayed low levels of fluorescence were retained and were dispensed into 96 well trays at approximately 1 cell per tray. Wells containing colonies were identified and the colonies were expanded.

Flow cytometry and microscopy. Flow cytometry was performed using FACScalibur and Cellquest software.

Results

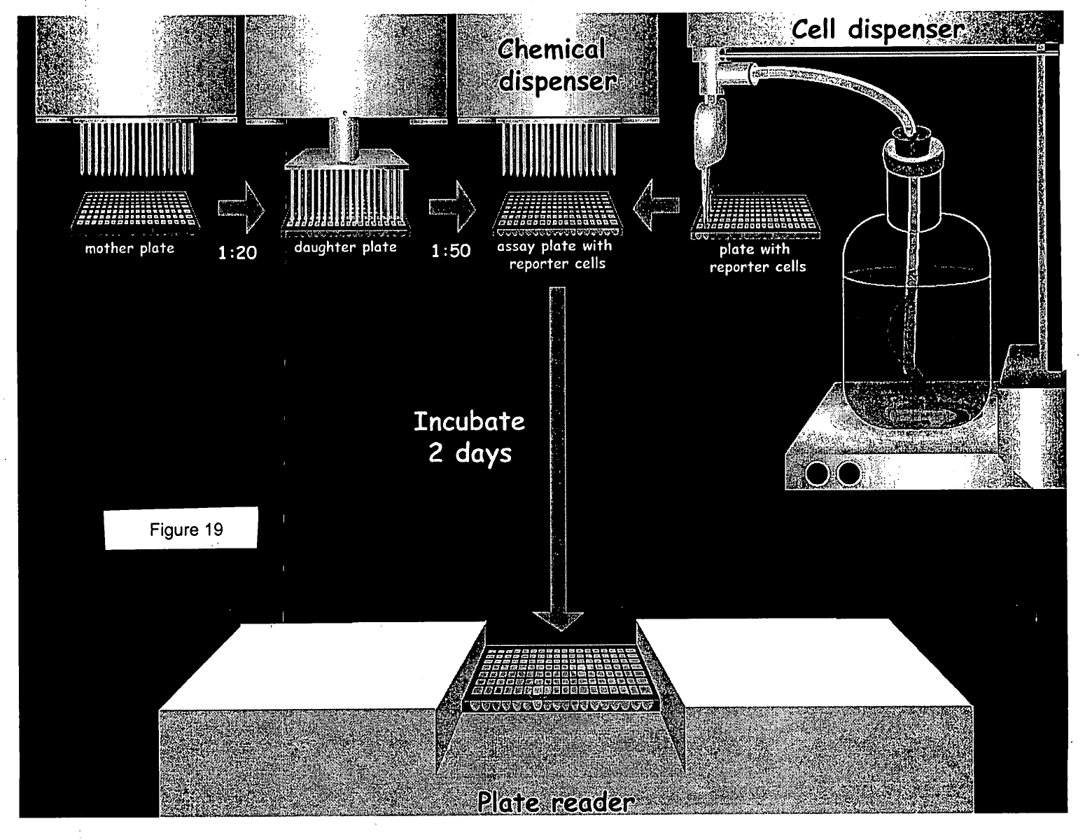

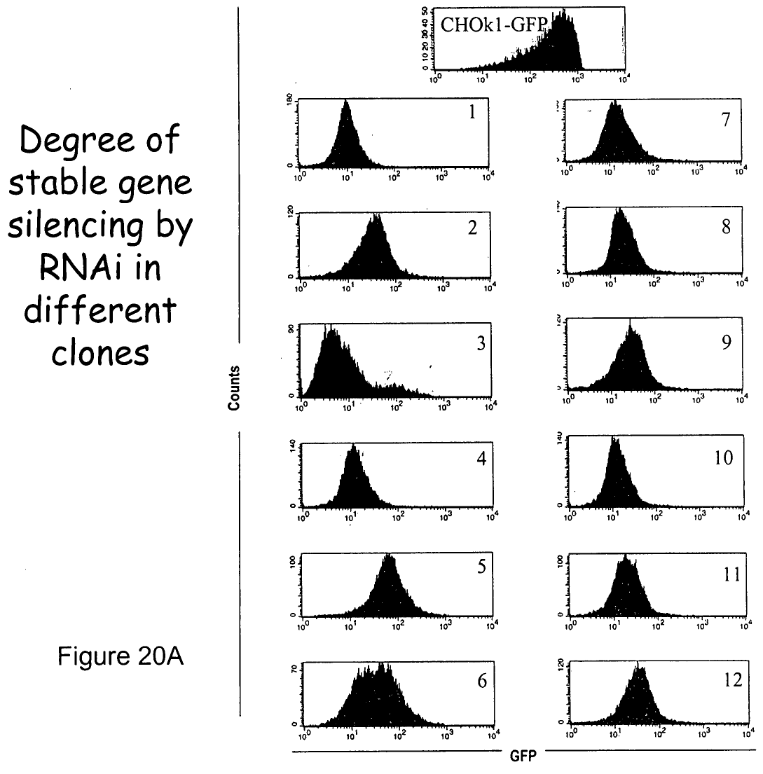

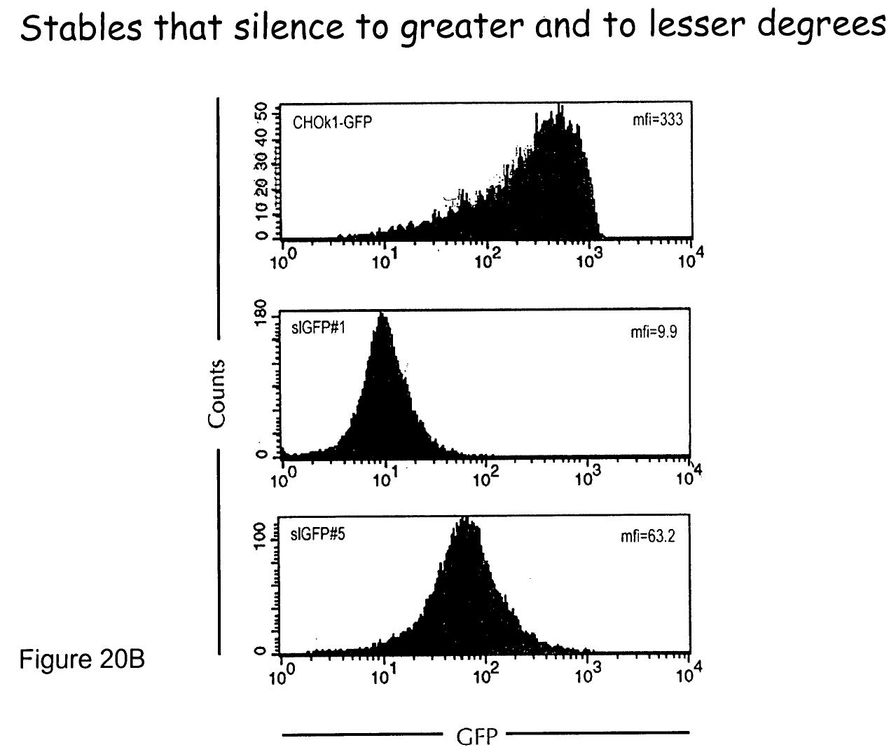

FIG. 20A shows flow cytometry results illustrating mean GFP fluorescence intensity in a parental cell line, CHOk1-GFP, that expresses GFP and in 12 clonal CHOk1-GFP-shGFP cell lines derived from the parental cell line and expressing a shRNA targeted to the GFP protein, in which GFP expression is silenced to differing degrees. The single cell cloning strategy revealed significant heterogeneity among different clonal populations. For example cell line 1 exhibits a single narrow and symmetric peak without a significant tail at either end, whereas cell line 7 displays a broader peak. Cell line 3 exhibits a large peak with low mean fluorescence intensity and a considerably smaller peak with a higher mean fluorescence intensity, suggesting that this clone contains two populations of cells. Cell line 5 exhibits a single relatively sharp peak with a significantly higher mean fluorescence intensity than cell line 1, suggesting that silencing by RNAi in this cell line is less strong than in cell line 5. FIG. 20B provides a quantitative indication of the difference in mean fluorescence intensity in cell lines 1 and 5 and the overall magnitude of silencing relative to the parental cell line. The mean fluorescence intensity of the parental cell line was 333 while the mean fluorescence intensity of cell line 1 was 9.9, indicating an 33 fold decrease in GFP expression as measured by fluorescence intensity. The mean fluorescence intensity of cell line 5 was 63.2, indicating an ˜5.3 fold decrease in GFP expression as measured by fluorescence intensity. Thus the strength of the RNAi silencing effect varies by a factor of approximately 6 between these two cell lines.

While not wishing to be bound by any theory, it is likely that the finding that clonal cell lines derived from the same parental cell line exhibit variability in RNAi has a number of implications with respect to performing genetic screens and selections for RNAi pathway mutants and/or screens or selections for identification of chemical modulators of RNAi pathway components. For example, in order to minimize the number of false positives it may be preferable to utilize a cell line that exhibits a single sharp peak of GFP fluorescence. In order to identify gain of function mutations or chemical activators of RNAi, it may be preferable to utilize a cell line that exhibits a weak RNAi phenotype relative to other cell lines so that it will be easier to detect an increase in the strength of the RNAi phenotype. Conversely, in order to identify loss of function mutations or chemical inhibitors of RNAi, it may be preferable to utilize a cell line that exhibits a strong RNAi phenotype relative to other cell lines so that it will be easier to detect a decrease in the strength of the RNAi phenotype.

### Example 3

Reduction in stable gene silencing by RNAi-mediated inhibition of Dicer.

Experimental Procedures

Flow cytometry and microscopy. Flow cytometry was performed using FACScalibur and Cellquest software. Microscopy and image acquisition were performed using an Axioplan 2 microscope (Zeiss) and Axiovision Viewer 3 software (Zeiss).

Vector construction. To construct a vector (pRLL-shDCR) for silencing Dicer expression, an inverted repeat sequence with a stem sequence whose antisense strand is perfectly complementary to a portion of the human Dicer gene, followed by a five thymidine repeat sequence, was cloned 5′ of the central polypurine tract (cPPT) from pRLL-cPPT-hPGKEsin (Dull, T., et al., J. Virol., 72: 8463-8471, 1998) using the following oligomers:

| | |
|---|---|
| DCR-1 (sense): | |
| 5′-AATTCCCTCAACCAGCCACTGCTGGATTCAAGA | (SEQ ID NO: 3) |
| GATCCAGCAGTGGCTGGTTGATTTTTCTCGAG-3′ | |
| DCR-2 (antisense): | |
| 5′-GATCCTCGAGAAAAATCAACCAGCCACTGCTGG | (SEQ ID NO: 4) |
| ATCTCTTGAATCCAGCAGTGGCTGGTTGAGGG-3′ | |

A cPPT was inserted 5′ of the human phosphoglycerate kinase (hPGK) promoter in the pRRL-hPGKsin vector. The puromycin resistance gene was cloned 3′ of the hPGK promoter.

Virus production and infection. Lentivirus (Lenti-shDCR) for silencing Dicer expression was produced by cotransfection of 293T cells with pRLL-shDCR, pHCMVG (Burns, J. C., et al., Proc. Natl. Acad. Sci., 90: 8033-8037, 1993) and pCMVΔR8.20vpr (An, D. S., et al., J. Virol., 73: 7671-7677, 1999). Transfections were carried out using Fugene 6 (Roche). Virus was harvested at 48 and 72 h posttransfection and infections were carried out in the presence of 10 mg/ml polybrene and 10 mM HEPES. Following transduction, cells were selected with 1 μg/ml puromycin.

Results

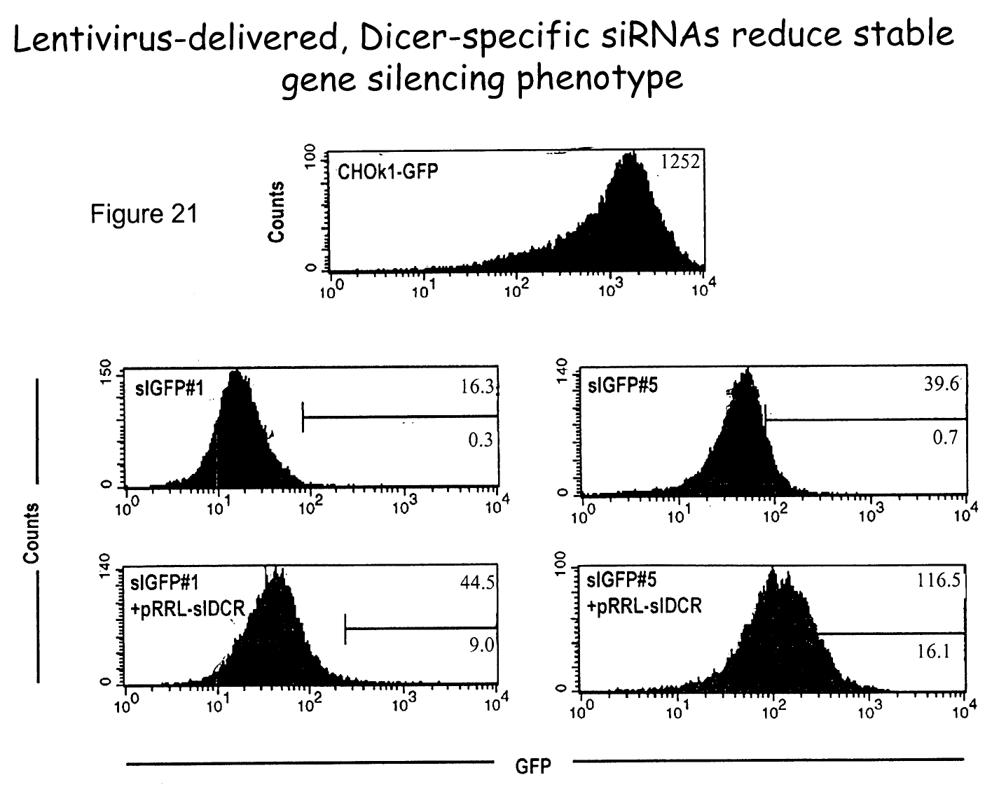

FIG. 21A shows FACS analysis of a CHOk1 cell line expressing GFP (CHOk1-GFP) and exhibiting a high fluorescence intensity. FIGS. 21B and 21C show silencing of GFP in two clonal cell lines (CHOk1-GFP-shGFP#1 and CHOk1-GFP-shGFP#5) derived from the parental CHOk1-GFP cell line and stably expressing an shRNA targeted to GFP. As shown in FIGS. 21A and 21B, expression of the shRNA greatly decreased GFP expression as demonstrated by the diminished mean fluorescence intensity (9.9 and 39.6 in clones 1 and 5 respectively) versus the fluorescence intensity in the parental cell line (1252). (Fluorescence intensity is expressed in arbitrary units).

In order to determine whether inhibiting the RNAi pathway that leads to silencing of GFP by the shGFP shRNA would result in a detectable decrease in silencing, expression of the Dicer enzyme, which is needed for the processing of shRNA to generate the active siRNA species, was inhibited by infection with the Lenti-shDCR lentivirus, which provides a template for transcription of an shRNA targeted to Dicer. FIGS. 21D and 21E illustrate the effects of inhibiting Dicer expression on the ability of the shGFP hairpin to silence GFP expression. As shown in these figures, inhibition of Dicer resulted in an increase in mean fluorescence intensity from 16.3 to 44.5 in cell line 1 and from 39.6 to 116.5 in cell line 5, indicating that inhibition of silencing (e.g., via a mutation in an RNAi pathway gene or via a chemical inhibitor) can result in a detectable increase in GFP expression.

It will be appreciated that since inhibition of Dicer was achieved by delivery of an shRNA targeted to Dicer, effective Dicer inhibition requires that the RNAi pathway retain some level of activity. In other words, while not wishing to be bound by any theory, it is likely that the strategy of inhibiting RNAi by delivering an RNAi-inducing agent targeted to an RNAi pathway component results in only a partial inhibition of silencing, i.e., an equilibrium is established in which the extent to which an RNAi-inducing agent targeted to an RNAi pathway component can inhibit the RNAi pathway is limited because the efficacy of the agent will decrease with increasing inhibition of the RNAi pathway (the inhibitory agent will no longer silence as the RNAi pathway is inhibited). This suggests that inhibiting the RNAi pathway by means other than via RNAi itself (e.g., by causing a loss-of-function mutation in an RNAi pathway gene, by overexpressing a dominant inhibitor of an RNAi pathway component, by inhibiting expression of an RNAi pathway component by an antisense strategy that is independent of the RNAi pathway, or by exposing cells to a chemical inhibitor of an RNAi pathway component), an even greater increase in GFP expression will be achieved, thereby facilitating detection of RNAi pathway mutants and inhibitors (see Examples below).

In conclusion, this example shows that inhibition of a gene involved in RNAi pathways results in a detectable reduction in RNA-mediated gene silencing, thus demonstrating the feasibility of (i) isolating mutants with loss of function mutations in genes involved in RNAi in mammalian cells; (ii) isolating dominantly acting repressors of RNAi; or (iii) isolating chemical inhibitors of RNAi using a screen based on an increase in GFP fluorescence in a cell line in which GFP expression is inhibited by RNAi. Conversely, mutants with gain of function mutations in genes involved in RNAi and genetic or chemical activators or RNAi can be identified by screening for cells with decreased GFP expression in a cell line in which GFP expression is not inhibited or is only partially inhibited despite the presence of an RNAi-inducing agent targeted to GFP within the cell.

### Example 4

siRNA-Mediated Silencing of HPRT Expression Allows Cell Growth Under Selective Conditions

Experimental Procedures

siRNA preparation. siRNAs with the following sense and antisense sequences were used (where the presence of a phosphate at the 5′ end of the RNA is indicated with a P):

| | |
|---|---|
| HPRT (sense) | |
| 5′-P.GUGUCAUUAGUGAAACUGGAA-3′ | (SEQ ID NO: 5) |
| HPRT (antisense) | |
| 5′-P.CCAGUUUCACUAAUGACACAA-3′ | (SEQ ID NO: 6) |

All siRNAs were synthesized by Dharmacon Research (Lafayette, Colo.) using 2′ACE protection chemistry. The siRNA strands were deprotected according to the manufacturer's instructions, mixed in equimolar ratios and annealed by heating to 95° C. and slowly reducing the temperature by 1° C. every 30 s until 35° C. and 1° C. every min until 5° C.

siRNA transfection. HeLa cells were trypsinized and plated in 6 cm wells at 1×10⁵ cells per well for 12-16 h before transfection. Cationic lipid complexes, prepared by incubating 100 pmol of indicated RNA with 3 ul oligofectamine (Gibco-Invitrogen, Rockville, Md.) in 100 ul DMEM (Gibco-Invitrogen) for 20 min, were added to the wells in a final volume of 1 ml. Cells were transfected overnight, washed and resuspended in fresh medium.

RT-PCR. mRNA was isolated from cells using standard techniques. RT-PCR amplification of β-actin and HPRT mRNA was performed using standard techniques using primers specific for β-actin and HPRT respectively. Amplification products were run on an agarose gel.

Selection. For 8-azaguanine (8-AZ) selection, HeLa cells were grown in DMEM containing 8-AZ from Sigma at ˜3.3×10⁻⁶ M for selection.

Results

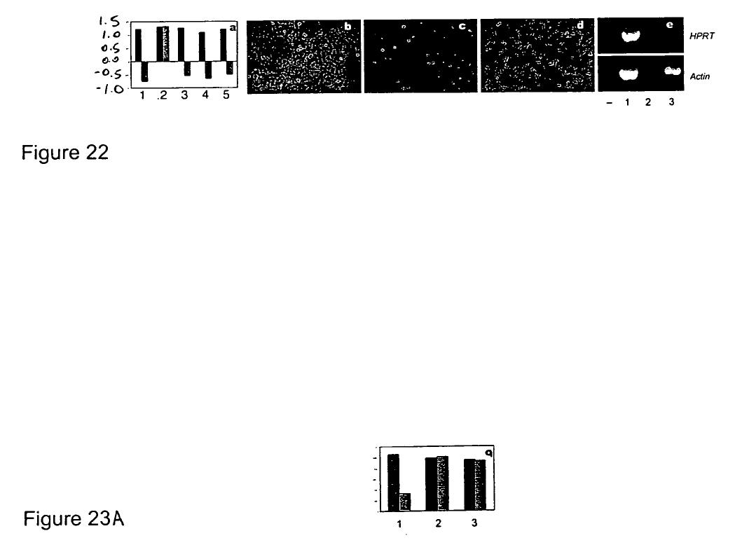

This experiment allowed us to determine that silencing of HPRT expression by RNAi is sufficient to allow cell growth in selective media containing a compound such as 8-AZ, which is metabolized to a toxic compound by the HPRT enzyme. FIG. 22A shows a growth rate comparison between HeLa cells grown in DMEM (black bars) or in DMEM containing 8-AZ (red bars). HeLa cells expressing wild type levels of HPRT were either mock transfected (1), transfected with siRNA targeted to HPRT (2), or transfected with either the sense strand (3) or the antisense strand (4) of this siRNA or with a nonspecific control (5). As indicated in the figure, cells with wild type HPRT expression die in medium containing 8-AZ while transfection of siRNA targeted to HPRT allowed growth of such cells at wild type levels. Transfection of either sense or antisense strands of the HPRT-specific siRNA failed to allow growth. FIGS. 22B and 22C are photomicrographs of HeLa cells grown in DMEM (22B) or DMEM+8-AZ (22C) showing that failure of the cells expressing HPRT to grow in 8-AZ can readily be detected microscopically. FIG. 22D is a photomicrograph of HeLa cells transfected with siRNA targeted to HPRT and grown in medium containing 8-AZ, showing robust growth of the cells. The cells were transfected with the siRNA twice, with the second transfection taking place two days after the first. 8-AZ was added the day after the second transfection. Photos were taken on the third or fourth day after addition of 8-AZ.

Reverse transcription (RT)-PCR of HPRT mRNA was performed to confirm that silencing in this system is a post-transcriptional effect. FIG. 22E shows RT-PCR using primers for amplification of HPRT or β-actin control from mRNA isolated from HeLa cells grwon in DMEM (1) or in DMEM containining 8-AZ (2) or from HeLa cells transfected with siRNA targeted to HPRT and grown in 8-AZ (3). The (−) denotes the no reverse transcriptase control.

These data demonstrate that HPRT silencing requires the siRNA duplex, is sequence-specific, is not an “antisense” effect, and permits growth of HeLa cells in DMEM containing 8-AZ that is microscopically indistinguishable from growth in the absence of 8-AZ selection. The data further indicate that the ability of cells in which HPRT expression is silenced by RNAi to grow in selective media can be used to identify mutants in RNAi pathway genes and/or genetic or chemical inhibitors of RNAi pathways.

### Example 5

Effects of Differential HPRT Expression on Cellular Growth Rates Under Selection for or Against HPRT Expression

Experimental Procedures

CHO cell lines. CHOk1 is a Chinese hamster ovary cell line expressing wild type levels of HPRT. 5A9 is a CHO cell line expressing a low level of HPRT. A563 is a CHO cell line with undetectable HPRT expression. These cell lines were obtained from L. A. Chasin, Columbia University and have been well characterized previously.

Selection. For 6-thioguanine (6-TG) selection, CHO cells were grown in 6-well multiwell plates in F-12 medium or in F-12 containing 6-thioguanine (6-TG) (at 1× from a 50×stock solution, Sigma catalog no. A4660). HAT selection was performed in F-12 containing 1×HAT (from a 50×stock solution, Sigma catalog no. H-0262). Multiple replicate wells were used for each selection condition to allow multiple measurements and determination of cell number at various time points.

Quantitation of cell number. Cell number at each time point was determined by trypsinizing cells in individual wells and resuspending in a volume of 1 ml. Cells were counted using a Coulter counter. Growth rate was computed by calculating the best fit line (in the least squares sense) of log (cell number) as a function of time and then using the following equations:

Slope=[(log cell number at t₁)−(log cell number at t₀)]/(t₁−t₀)

Doublings=[(log cell number at t₁)−(log cell number at t₀)]/log 2

Doublings/time=Slope/log 2

Results

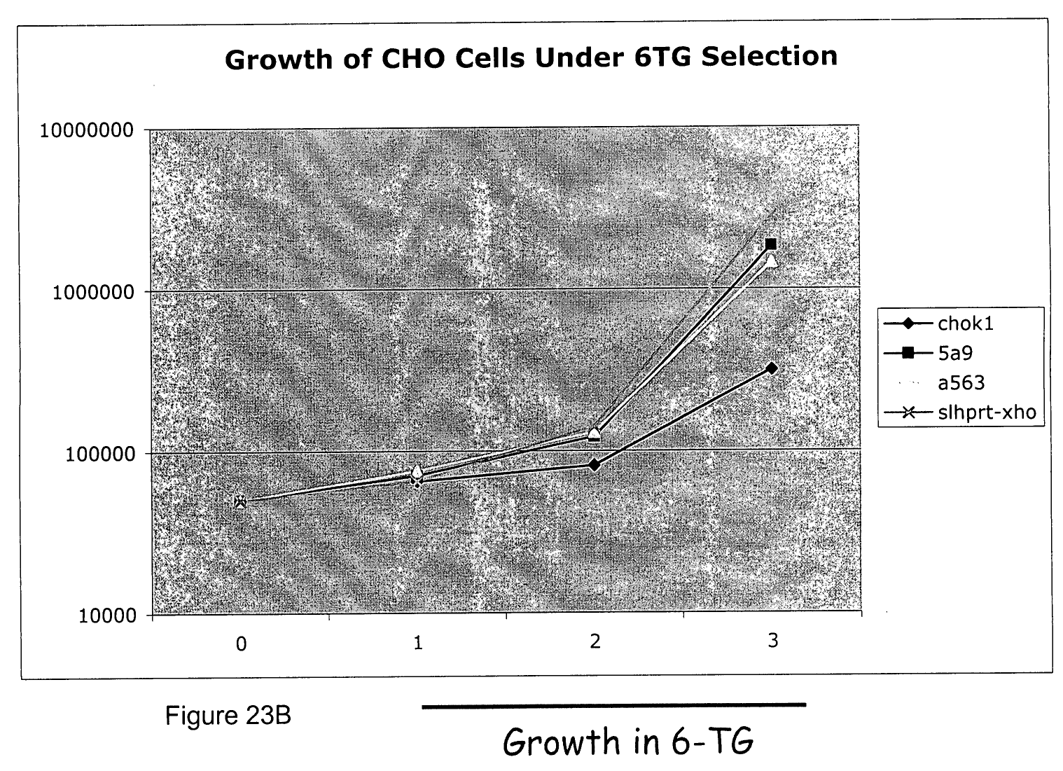

To determine the effects of differential HPRT expression on cellular growth rates (i.e., cellular division rate) under conditions that select against cells that express HPRT, CHO cell lines expressing varying amounts of HPRT were grown in either F-12 medium or F-12 medium containing the compound 6-thioguanine (6-TG) which, like 8-AZ, is metabolized to a toxic compound by HPRT. FIG. 23A shows growth rates of three CHO cell lines with wild type level (CHOk1), low level (5A9), or undetectable (A563) HPRT expression in F-12 medium (black bars) or F-12 medium containing 6-TG (red bars), expressed as doublings per hour. The presence of 6-TG caused a significant reduction in growth rate in cells expressing wild type HPRT levels, while a reduction in HPRT levels allowed cells to grow at the same rate in the presence of 6-TG as in its absence. FIG. 23B shows the growth of the three cell lines under 6-TG selection as a function of time.

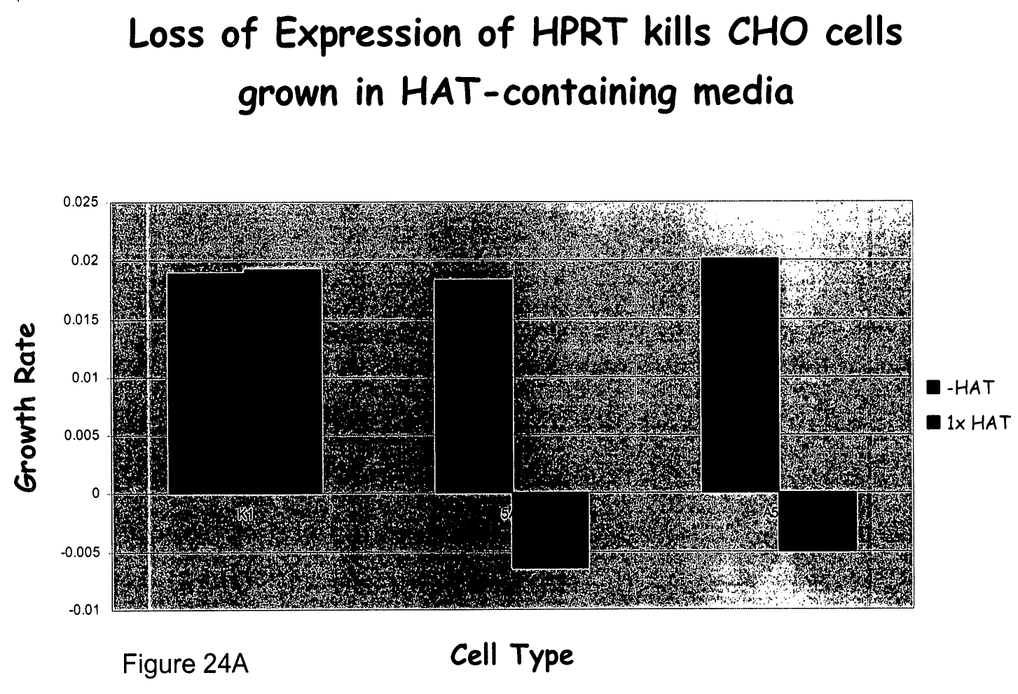

To determine the effects of differential HPRT expression on cellular growth rates under conditions that select for cells that express HPRT, CHO cell lines expressing varying amounts of HPRT were cultured in either F-12 medium or F-12 medium containing the compounds hypoxanthine, aminopterin, and thymidine (HAT). As discussed above, aminopterin inhibits dihydrofolate reductase (DHFR), a key enzyme in the pathway for de novo synthesis of purines, which are required for DNA synthesis and thus for cell proliferation. Mammalian cells can survive in the absence of DHFR activity by utilizing a purine salvage pathway in which hypoxanthine is converted to inosine monophosphate (IMP) by HPRT. FIG. 24A shows growth rates of the same three CHO cell lines with varying levels of HPRT expression in F-12 (black bars) or in F-12 containing HAT (red bars). Cells with wild type HPRT expression were able to proliferate in medium containing HAT at a rate that was indistinguishable from that of cells in normal medium, while cells with reduced or absent HPRT expression failed to divide and declined in number. FIG. 24B shows the growth of the three CHO cell lines under HAT selection as a function of time.

### Example 6

Expression of shRNA Targeted to HPRT Results in Reversal of Growth Phenotypes under Selective Conditions

Experimental Procedures

Cell culture, transfection, selection, and quantitation. CHOk1 cells were cultured in F-12 medium or in F-12 medium plus HAT, at the concentration described above, for selection. To obtain a cell line that stably expressed a shRNA targeted to HPRT, CHOk1 cells were transfected with the plasmid pSHARP-shHPRT, described in Example 1, using standard techniques, and selected in medium containing 6-TG as described above. Cells were counted using a Coulter counter.

Results

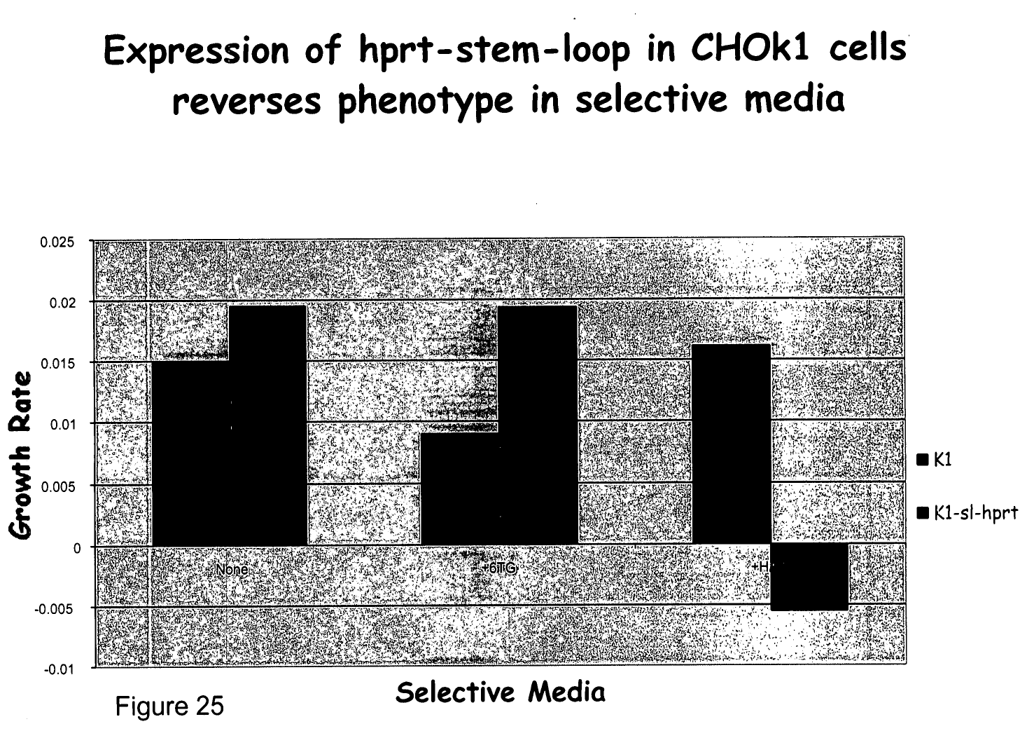

FIG. 25 shows growth rates of wild type CHOk1 cells (black bars) or wild type CHOk1 cells that stably express the shRNA targeted to HPRT. Wild type cells and cells expressing the HPRT shRNA grow in F-12 medium (1). However, CHOk1 cells that stably silence HPRT are able to grow at a significantly higher rate in medium containing 6-TG than cells expressing wild type levels of HPRT. CHOk1 cells that stably silence HPRT die when cultured in medium containing HAT, whereas cells with wild type HPRT expression are able to live in this medium. Growth rates are in terms of doublings per hour. Growth rates over time for these cells are also shown on FIGS. 23B and 24B (shHPRT-cho).

These data demonstrate that enhanced growth in the presence of a compound such as 6-TG that is toxic when a gene whose product confers sensitivity to the compound is expressed can be used to select for cells that have an intact RNAi pathway or to select for cells that have an enhanced RNAi pathway. The data further suggest that cells that have mutations in an RNAi pathway component, or in which RNAi is inhibited by either genetic or chemical means, can be selected under conditions in which cell viability and/or proliferation require expression of a particular gene (e.g., HPRT), whose expression can be silenced by an RNAi-inducing agent.

### Example 7

Identification of Compounds That Inhibit or Activate RNAi Pathways

Experimental Procedures

For screens based on GFP fluorescence, CHOk1-GFP-shGFP#1 cells stably expressing a shRNA targeted to GFP were seeded in F-12 medium in black, clear-bottom tissue culture-treated 384-well plates (Costar #3712, VWR#29444-078). Cells were treated with compounds from an annotated compound library (ACL), at concentrations ranging from 0-135 μg/ml. The ACL is described in Root, D. E., et al., Chemistry & Biology, 10:881-892 and is discussed above.

To identify cells that expressed GFP, cells were maintained in culture at 37° C. with 5% CO₂ for 2 days after which total fluorescence intensity of each well was measured using a 384 well plate reader spectrophotometer. Each compound plate was tested in triplicate. For each plate a “trimmed mean” was calculated by discarding approximately the top 5% and bottom 5% of values and calculating the average. We also calculated a trimmed mean locally for each well. That is, for each well we calculated the average of that well and the surrounding 8 wells. We discarded the highest and lowest values and calculated the average of the remaining 7 wells. The absolute value of each well was divided by the trimmed mean locally as well as by the trimmed mean for the plate. Then each well was averaged over the three replicates. Finally we set a rank order for each of the wells. Positives were the wells that recovered approximately 30% of the wildtype flourescence (the flourescence that was detected with cells that did not contain the hairpin targeted to GFP added to the the cells). A total of approximately 30-40 wells were identified as positives based on these criteria.

The compounds were retested by determining whether they were able to allow CHOk1-shHPRT cells to grow in HAT medium. To do so, CHOk1-shHPRT cells stably expressing a shRNA targeted to HPRT were seeded in F-12 medium (at concentrations described above) in black, clear-bottom tissue culture-treated 384-well plates (Costar #3712, VWR#29444-078). Compounds were added to the wells at concentrations that had scored positive in the GFP-based screen and HAT was then added at concentrations described above. Cells were then maintained in culture for 3 days. Cells were then washed 6 times with phosphate-buffered saline (PBS) and incubated with alamar blue (U.S. Pat. No. 5,501,959; Ahmed, S. A., et al. Immunol Meth 170:211-224, 1994), a nontoxic compound that is converted from blue to pink or red in the cytoplasm, according to the directions of the manufacturer (BioSource International). Total fluorescence intensity was measured on the Packard Fusion plate reader and percent inhibition of cell viability was calculated by subtracting instrument background and dividing by the average signal from untreated control cells. Other methods of determining cell viability, e.g., the BD™ oxygen biosensor system, MTT assay, lactate dehydrogenase assay, calcein-based assay (Root, et al.), etc., could also have been used.

Results

To identify compounds that inhibit RNAi pathways, CHOk1-GFP-shGFP cells, which stably express a shRNA targeted to GFP that silences GFP expression were dispensed into individual wells and exposed to various concentrations of compounds from an annotated compound library (ACL). Wells that displayed increased fluorescence in the presence of the compound, suggesting an inhibition of RNAi, were identified as follows:

Relative fluorescence intensity of each well was measured on a Packard Fusion plate reader with a 485 nm excitation filter (20 nm bandpass) and a 530 nm emission filter (25 nm bandpass). The fluorescence from each test-compound treated well was normalized to the average of fluorescence values from: (1) all of the wells on the plate, excluding the top two and bottom two rows and first and last columns on the plate and excluding from remaining wells those with the highest 20% and lowest 20% of fluorescence values. (2) the 8 wells surrounding the test compound being evaluated, excluding those with the highest two and lowest two fluorescence values. Three replicates of each plate were tested. The 3 values for each compound were averaged. Compounds showing a 6% or greater increase in fluorescence were applied to non-GFP expressing HeLa cells to make sure that the observed fluorescence increase was related to increase in GFP expression and not other factors such as intrinsic fluorescence of the test compound. A total of approximately 30-40 compounds scored positive in this GFP-based screen.

The compounds were retested to determine whether they would also allow growth in HAT medium of CHOk1-shHPRT cells, which stably silence HAT expression. Similar methods are used to perform an initial selection to identify compounds that inhibit RNAi. For each drug treatment, an AlamarBlue fluorescence assay was used to assess the % rescue of drug treated cells under HAT selection versus cells treated with HAT alone (set at 0% rescue). This % rescue was normalized to the cell viability in the absence of HAT selection (100% rescue). For each 384-well plate, two control groups were included:

(1) 16 wells were untreated, (2) 16 wells were treated with HAT but no additional test compound. 320 wells of the remaining wells were treated with HAT plus a test compound. An Alamar Blue assay was performed according to the manufacturer's directions. Relative fluorescence intensity of each well was measured on a Packard Fusion plate reader with a 530 nm excitation filter (25 nm bandpass) and a 590 nm emission filter. For the control groups, the two highest and two lowest measurements were dropped and the remaining 12 readings were averaged to obtain:

P=trimmed mean fluorecence of untreated cells

N=trimmed mean fluorecence of cells treated with HAT alone

The % rescue of cells from HAT selection (R) was measured by:

R=(D-N)/(P-N)

Three replicates of each plate were tested. The 3% rescue values R were averaged to obtain R(ave). For each treatment for which the % rescue was above ˜6-7%, the cells were observed by microscope to visually confirm increased viability. This served to confirm that the apparent rescue by fluorescence measurement was not simply due to background fluorescence from the test compound. In all cases the cells had normal cell boundaries, no cytoplasmic inclusion bodies and no visible signs of apoptotic death.

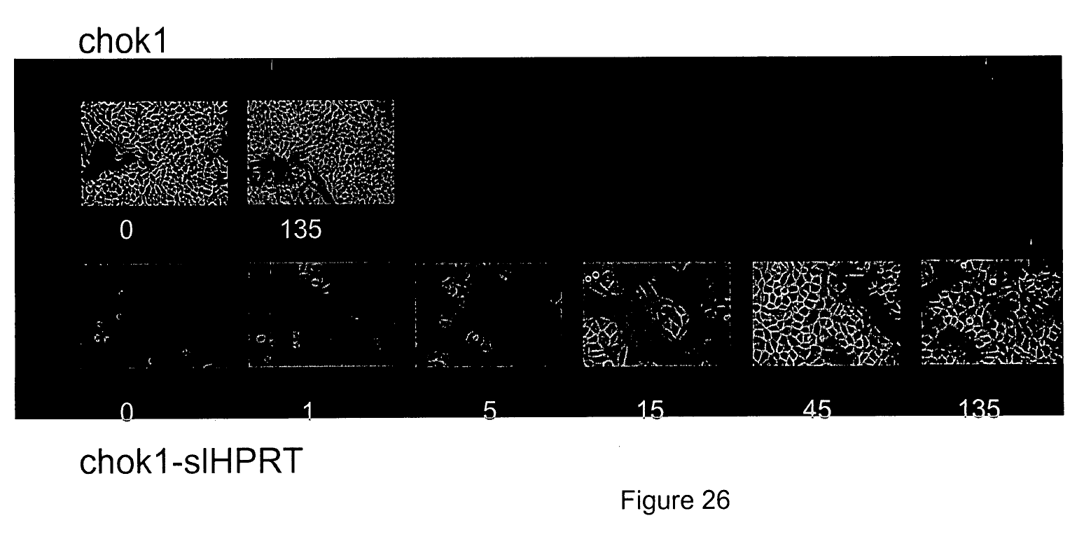

FIG. 26 shows a representative example of results with one of the identified putative RNAi inhibitors, 5′-AMPS. The lower panels show growth of CHOk1-shHPRT cells in F-12 medium plus HAT in the presence of increasing concentrations of 5′-AMPS. The images illustrate that increasing concentrations of the compound allow cells to grow in the presence of HAT, suggesting that the compound inhibits silencing of HPRT by the shRNA. The upper panels show growth of wild type CHOk1 cells in F-12 medium in the absence (left) or presence (right) of the compound, demonstrating that is is nontoxic even at the highest concentration tested. Numbers represent concentration of compound in the medium in μg/ml. Photos were taken after 3 days of growth in the presence of varying concentrations of compound.

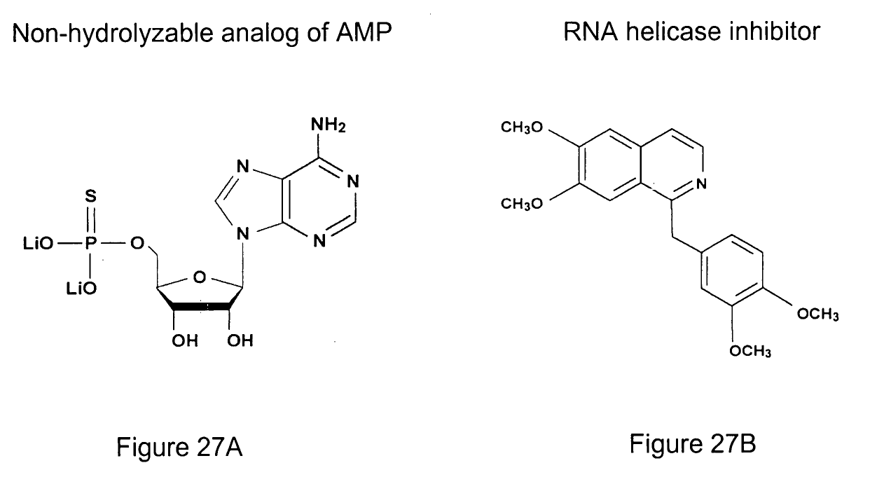

Since the compounds in the ACL have all been assigned biological mechanism discriptors, it was possible to divide the compounds that scored positive in the preliminary GFP-based based screen described above into a number of different classes. In particular, one or more compounds classified in the following functional categories (See Root, et al.), were identified using the GFP-based screen: (i) topoisomerase inhibitors; (ii) phosphodiesterase inhibitors; (iii) antibiotics; (iv) monoamine oxidase inhibitors; (v) RNA helicase inhibitor (see FIG. 27B); (vi) non-hydrolyzable analogs of purines (see FIG. 27A); (vii) antimalarial drug.

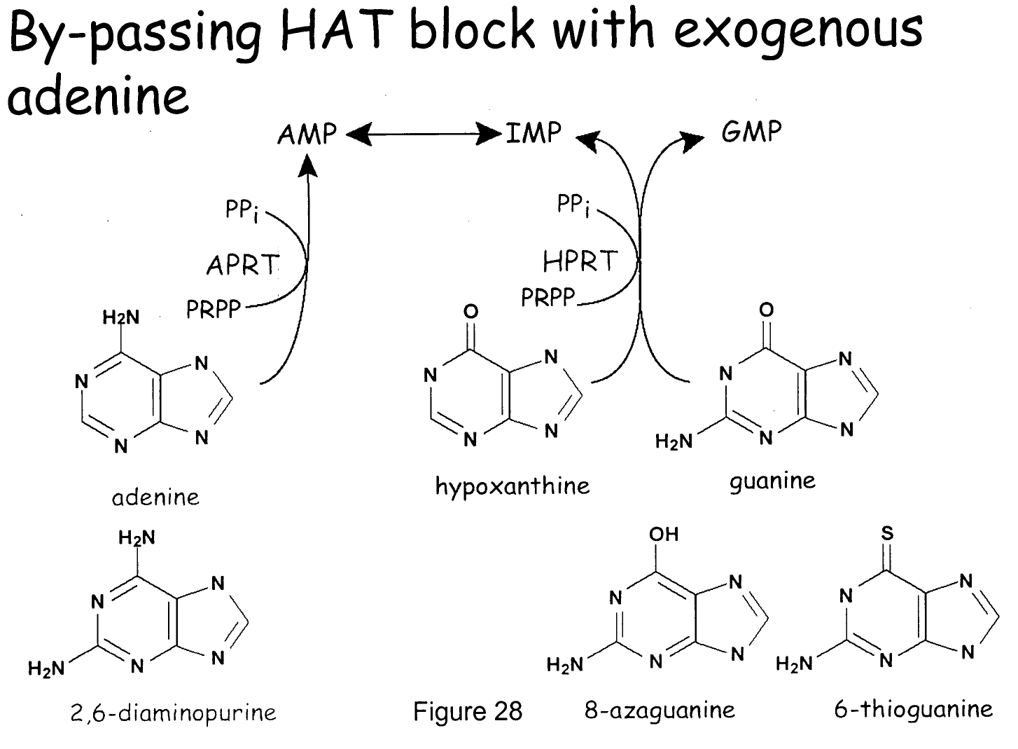

Given the functional/mechanistic information associated with each compound, it is possible to explore and test hypotheses regarding mechanisms by which they might act to inhibit RNAi. The availability of two different systems, based on two independent markers GFP and HPRT facilitates distinguishing among alternative hypotheses and rapidly eliminating false positives or compound classes (e.g., fluorescent compounds) that may be expected to systematically result in erroneous identification of a compound as an inhibitor of RNAi when in fact the compound acts by a different mechanism). For example, adenosine 5′-O-thio monophosphate (5′AMPS), shown in FIG. 27A, one of the compounds that scored positively in the preliminary GFP-based screen and on retest using the HPRT-based screen (i.e., CHOk1-shHPRT cells were able to grow in HAT medium in the presence of the compound), is a non-hydrolyzable analog of ATP. As mentioned above, at least one activity of RISC is known to be ATP-dependent. While not wishing to be bound by any theory, the inventors suggest that 5′AMPS may interfere with this ATP-dependent activity, possibly by binding to an ATPase present in RISC. Alternatively, it was observed that a number of analogs of adenosine were isolated in the HPRT-based selection for inhibitors of RNAi, i.e., these compounds allowed growth of cells in HAT medium, suggesting that they inhibited RNAi-mediated silencing of HPRT. However, it is also possible that these compounds, rather than inhibiting RNAi, allowed a bypass of the HPRT pathway according to the alternative pathway shown in FIG. 28, in which adenine phosphoribosyltransferase (APRT) converts adenine to AMP, which can then be converted into IMP. Six of the top 10 hits are adenosine or adenosine-derived analogs: methyaminopurine 9-ribofuranoside, N6-methyladenosine, S-adenosyl-L-methionine; N6-cyclopentyladenosine; adenosine 3′,5′-cyclic monophosphate; 5-Ethyl-2′-deoxyuridine; 2-Chloro-2-deoxyadenosine.

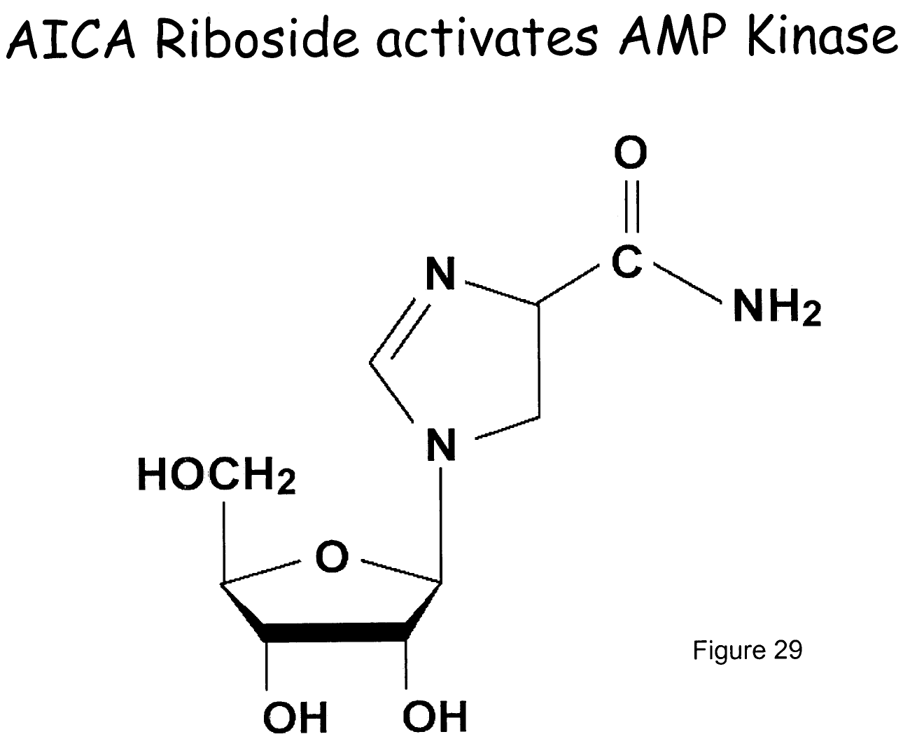

5′-Aminoimidazole-4-carboxamide riboside (AICA riboside), shown in FIG. 29, is known to activate AMP-activated protein kinase (AMPK), a molecule that is known to either activate or inhibit a variety of cellular processes and metabolic pathways (Kemp B E, et al., Trends Biochem Sci., 24(1):22-5, 1999). The finding that 5′AMPS inhibits RNAi suggests the possibility that the RNAi pathway is downstream of AMPK, e.g., that AMPK activity is needed for full activity of the RNAi pathway. This hypothesis may readily be tested by, for example, inhibiting or knocking out expression of AMPK (e.g., using an antisense RNA, using cells obtained from a mouse in which AMPK expression is knocked out as described in Viollet B, et al., Biochem Soc Trans., 31(Pt 1):216-9, 2003) and then determining whether cells in which AMPK expression is reduced or eliminated are able to support RNAi.

If AMP kinase positively regulates RNAi and if 5′AMPS inhibit AMP kinase OR if AMP kinase negatively regulates RNAi and 5′AMPS might activates AMP kinase then the effect of 5′AMPS can be explained without invoking the by-pass of HAT block. To test this, cells are treated with AICA riboside which activates AMP kinase, to determine whether that results in the same finding as 5′AMPS. In summary, initial selections and screens identified a number of compounds that inhibit RNAi. Use of the ACL permitted the generation of hypotheses regarding the possible mechanism of action of these compounds and methods for testing these hypotheses. Further work is being performed to confirm or exclude the candidates that initially tested positive in the screens described above.

### Example 8

A Genetic Approach to Identify RNAi Pathway Mutants

Experimental Procedures

Genetic suppressor element (GSE) library. To construct a genetic suppressor element library, total RNA was extracted from HeLa cells using standard techniques, poly(A)+ purified using an oligo-dT cellulose column, and fragmented by boiling for 5 min to reduce size. Double-stranded cDNA was synthesized, and Sfi I polylinkers were added. The resulting fragments were size fractionated, amplified, normalized, and inserted without regard to orientation into the multiple cloning site of the retroviral vector pLXSfi (FIG. 12) so that both sense and antisense orientations are represented in the library essentially as described (Gudkov, A. I., et al. Proc. Natl. Acad. Sci., 91: 3744-3748, 1994). The vector is a modification of pLXSN available from BD Biosciences (Clontech). The modified vector has Sfi sites in the multiple cloning site (rare cutters) and the cDNA fragments have Sfi linkers on both ends. The inserted fragments range in size from approximately 200 nucleotides to approximately 1 kB so that encoded protein fragments are more likely to contain only single functional domains. The library is either transfected into recipient cells, e.g., cells that express an RNAi-inducing agent that inhibits expression of a selectable or detectable marker, or are used to produce infectious virus as described below, which is then used to infect recipient cells.

For the experiments described below, a commercial GSE library, the ViraPort® XR plasmid cDNA library (Stratagene) was used. The ViraPort library contains human cDNAs directionally inserted into the retroviral vector pCFB. To produce infectious virus, the vector was packaged in 293T cells according to the directions of the manufacturer. Virus-containing supernatants harvested from the cells were used to infect CHO-shHPRT cells, which were then subjected to selection in HAT medium (HAT concentration as described above). More than 300 colonies able to grow in HAT medium were isolated and pooled. DNA was extracted from the pooled colonies and PCR was performed using an upstream primer to sequences in the 5′ end of the ViraPort library backbone and a downstream primer corresponding to the sequence of the gene encoding Dicer, the only factor known to be required for RNAi in mammalian cells. The PCR products were run on an agarose gel and bands were detected, indicating the presence of Dicer sequences in the pool. Conversely, when a downstream primer (to sequences in the 3′ end of the ViraPort library backbone) and a different primer to Dicer were used for PCR, bands of different lengths and of lower intensities were obtained, consistent with cDNA libraries with a preponderance of sequences from the 3′ portion of genes. Finally, using the two Dicer sequences for PCR a different band appeared relative to the other two. While not conclusive, these findings strongly suggest that at least one of the colonies selected using the GSE selection approach harbored a GSE that inhibited Dicer. It is noted that the ViraPort library comprises human cDNA sequences and the primers used corresponded to human Dicer, while the cellular DNA was from hamster (CHO cells), thus reducing the likelihood that the PCR product in fact represented amplification of endogenous CHO cell Dicer.

Individual cDNA inserts are recovered from the DNA using vector-specific primers and are subcloned for sequencing and for functional validation. Additional rounds of screening and/or selection using these subcloned cDNAs (GSEs) may be performed prior to, concurrently with, or following the sequencing. The cDNAs may be used as probes to obtain longer cDNA or genomic clones, either by probing the ViraPort library itself or by probing another cDNA or genomic library.

### Example 9

Creation and Testing of a Stable Cell Line to Identify Genes and Chemical Agents That Modulate miRNA Translational Repression Pathways

A reporter construct suitable for introduction into mammalian cells to create cell lines that can be used for identification of genes involved in miRNA translational repression pathways and/or chemical modulators of such pathways was created. Briefly, the coding sequence of firefly luciferase was restriction digested out of pGL3 (Promega) and inserted into pcDNA 3.1 (Invitrogen). This pcDNA vector drives expression from a CMV promoter and includes a BGH polyadenylation signal, although neither the identity of the promoter nor the polyA signal are critical to the ultimate assay. Six miR-21 binding sites were then inserted into the 3′ UTR region, resulting in a construct schematically depicted in FIG. 31A. The miR-21 binding sites were designed to have a bulge in the central region of the miRNA:mRNA interaction in order to mimic most known miRNA:mRNA interactions (Bartel, supra). FIG. 31B illustrates the interaction between miR-21 and a target binding site. The binding sites are separated by 4 nucleotides (CCGG), although experimental evidence suggests that the distance between the binding sites does not appear to be an important determinant of activity (Doench, J. and Sharp, P. A., Genes and Dev., 18(5):504-11, 2004). miR-21 was chosen because it is known to be an abundantly expressed miRNA in HeLa cells, i.e., at about 10,000 copies per cell.

This plasmid was linearized and transfected into HeLa cells, and G418 selection applied. Surviving cells were single-cell sorted, and individual colonies grown up. Cell culture, transfection, and single cell cloning were performed essentially as described above.

Colonies were screened for luciferase activity using standard methods to identify cell lines that stably expressed the reporter. It is noted that this screen identified cell lines in which the endogenous miR-21 only partially repressed translation of the luciferase mRNA. Such cell lines allow for identification of genes and chemical agents that act to either enhance or inhibit miRNA-mediated silencing. In parallel, a control cell line was made. This cell line differs in that the 3′ UTR in the reporter transcript has 6 binding sites that do not hybridize to any known miRNAs and responds to an exogenous siRNA targeting CXCR4 by silencing expression of the reporter via an miRNA-like interaction with the transcript (Doench, J., et al., Genes and Dev., 15;17(4):438-42, 2003). In other words, the CXCR4 binding sites are not perfectly complementary to the CXCR4 siRNA, but would form a bulge similar to the miR-21 bulge, such that the CXCR4 siRNA would cause translational repression rather than cleavage and is thus acting as an miRNA-like RNA. An additional control cell line containing a reporter transcript that contains binding sites for a CXCR4 siRNA that are perfectly or largely complementary to the siRNA antisense strand, such that siRNA would act via transcript cleavage rather than translational repression, is also constructed.

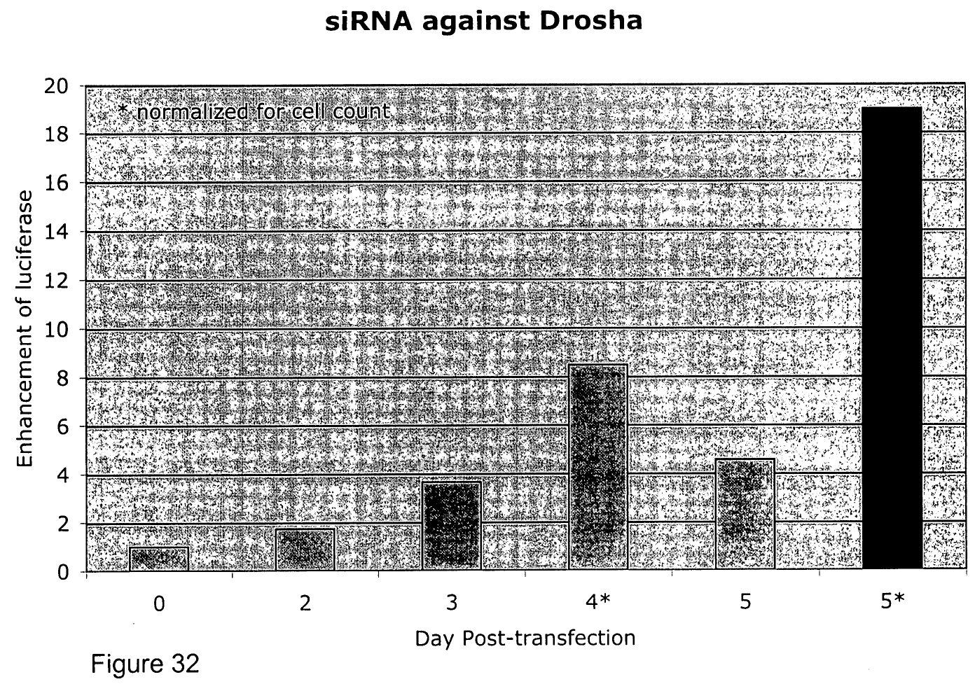

In order to demonstrate that the reporter was being silenced by miR-21, a representative cell line containing the reporter, referred to as miR-21-UTR was tested by transfecting the cell with an siRNA targeted to mRNA encoding Drosha, an RNAse III-like enzyme critical for processing miRNA precursors. Silencing of Drosha would be expected to inhibit miRNA biogenesis, thereby preventing miR-21-mediated translational repression. In the absence of translational repression, luciferase activity should increase. FIG. 32 is a time course in which luciferase activity is measured at various times following transfection of the siRNA targeted to Drosha into the miR-21-UTR cell line. As shown in the figure, by day 3, there is ˜4 fold increase in luciferase activity in the cell line as compared to the effect of transfection of a control siRNA (targeting GFP). By day 4, the Drosha knockdown caused a noticeable decrease in the growth rate of these cells, and thus the luciferase activity is normalized to the number of cells, as indicated by the *. By day 5, this effect starts to wane, but if one transfects the cells on both day 0 and day 3, by day 5, normalized to cell count (i.e. the day 5 bar with the star) one sees a ˜20 fold increase in luciferase activity. Transfecting cells with siRNAs more than once is a common procedure. The siRNA targeted to Drosha has been previously described (Lee, Y., et al., Nature, 425(6956):415-9, 2003). In these experiments, the siRNA were transfected with Oligofectamine (Invitrogen).

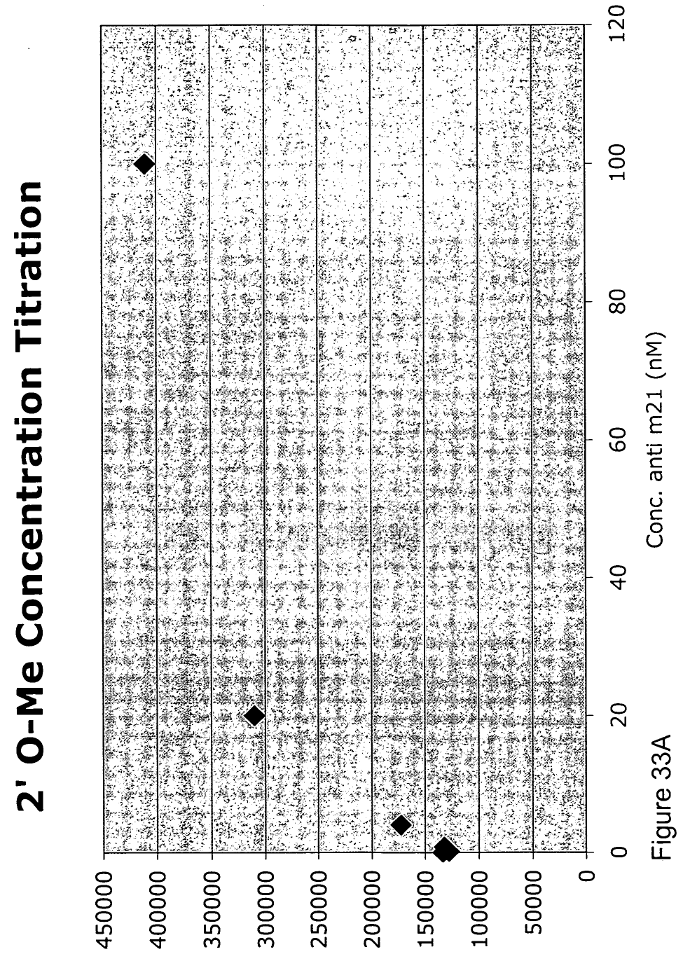

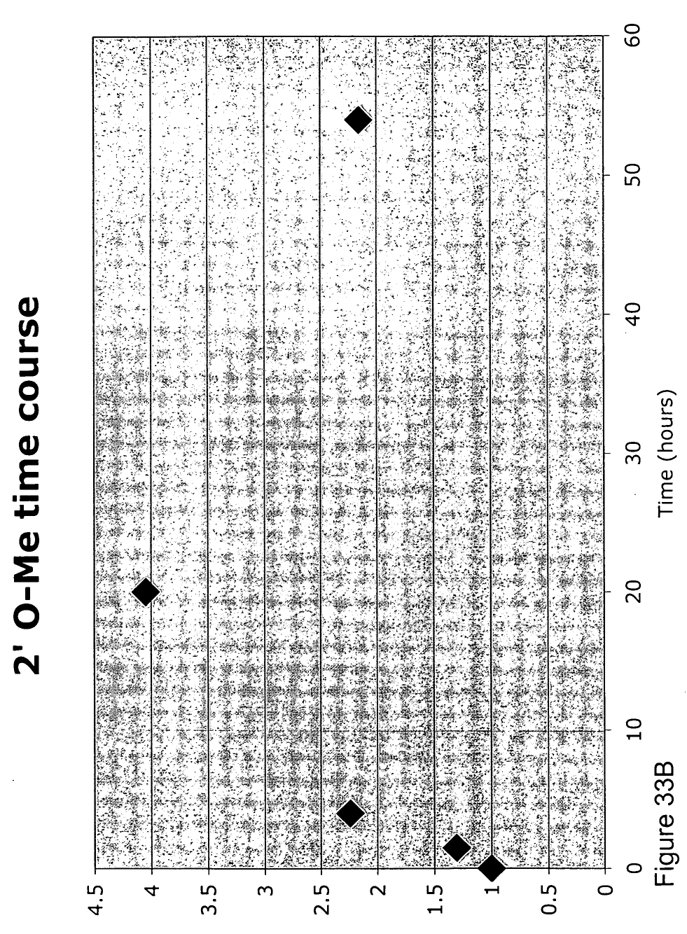

The experiment described above showed that the microRNA pathway was involved in silencing the luciferase activity in this cell line. To show that miR-21 is specifically involved in the silencing a series of experiments using 2′-O-Methyl modified RNA was performed. RNA molecules with a methyl group attached to the 2′ hydroxyl (i.e., 2′O-Me RNA) have been shown to bind to and thus inactivate siRNAs and miRNAs both in vitro and in vivo (Hutvagner G., et al., PLoS Biol. 2004 April; 2(4):E98. Epub 2004 Feb. 24). Thus, a RNA molecule with perfect complementarity to miR-21, with 2′O-Me modifications at each nucleotide, was synthesized (IDT) and transfected into the cells with Oligofectamine. Various concentrations of modified RNA oligonucleotide were transfected, and luciferase activity was assayed at 19 hours post-transfection. In FIG. 33A it can be seen that this 2′-O-Me RNA caused a decline in miR-21 mediated silencing of luciferase in a dose-dependent manner. Control experiments, in which an oligonucleotide of a different sequence was transfected, or in which the anti-miR-21-2′-O-Me, i.e., an RNA that is perfectly complementary to miR-21, was transfected into the control cell line described above (i.e, the control cell line that responds to an exogenous siRNA targeting CXCR4 by silencing expression of the reporter via an miRNA-like interaction with the transcript) showed no increase in luciferase acvitiy. FIG. 33B shows results of a similar experiment but performing a time course rather than a concentration titration. 100 nM oligo was used in this experiment. These results demonstrate that it is possible to detect genes and/or chemical agents that reduce miRNA translational repression pathways in the cell lines described above by detecting an increase in luciferase expression. Genes and/or chemical agents that increase miRNA translational repression pathways would be identified by detecting a decrease in luciferase expression. It will be appreciated that a wide variety of detectable markers could be used in a similar manner. The invention also encompasses the use of selectable markers for these purposes.

Additional cell lines that express an shRNA targeted to the luciferase mRNA in addition to the miR-21 miRNA were also created by transfecting a construct providing a template for transcription of the shRNA into the cells and selecting single cell clones. The sequence of the shRNA was

CTTACGCTGAGTACTTCGAAACTCGAGTTTCGAAGTACTCAGCGTAAGTTTTTTG (SEQ ID NO: 7), of which CTCGAG represents sequence of the Xho I loop.

Those skilled in the art will recognize, or be able to ascertain using no more than routine experimentation, many equivalents to the specific embodiments of the invention described herein. The scope of the present invention is not intended to be limited to the above Description, including specific embodiments described in the Examples, but rather is as set forth in the claims.

1. A mammalian cell comprising:
(i) a nucleic acid that encodes a selectable marker; and
(ii) one or more nucleic acid templates for transcription of an RNAi-inducing agent integrated into the genome of the cell, wherein the RNAi-inducing agent reduces expression of the marker and is not naturally found in the cell.

2. The cell of claim 1, wherein the cell is a human cell.

3. The cell of claim 1, wherein the cell is a HeLa cell.

4. The cell of claim 1, wherein the cell is a non-human cell.

5. The cell of claim 1, wherein the cell is hypodiploid.

6. The cell of claim 1, wherein the cell is a CHO cell.

7. The cell of claim 1, wherein the selectable marker is an expression product of an endogenous gene.

8. The cell of claim 1, wherein the nucleic acid that encodes a selectable marker is operably linked to an inducible promoter.

9. The cell of claim 1, wherein the nucleic acid that encodes a selectable marker is operably linked to a constitutive promoter.

10. The cell of claim 1, wherein the cell further comprises:
(i) a nucleic acid that encodes a detectable marker; and
(ii) one or more templates for transcription of an RNAi-inducing agent that reduces expression of the detectable marker.

11. The cell of claim 10, wherein a template for transcription of the RNAi-inducing agent that reduces expression of the detectable marker is integrated into the genome of the cell.

12. The cell of claim 1, 10, or 11, wherein the RNAi-inducing agent is a short hairpin RNA (shRNA).

13. The cell of claim 1, 10, or 11, wherein the RNAi-inducing agent is an siRNA or precursor thereof.

14. The cell of claim 1, 10, or 11, wherein the RNAi inducing agent is a precursor of a microRNA (miRNA).

15. The cell of claim 1, 10, or 11, wherein the cell further comprises:
(i) a nucleic acid that encodes a second selectable marker; and
(ii) one or more templates for transcription of an RNAi-inducing agent that reduces expression of the second selectable marker.

16. The cell of claim 10 or 15, wherein the detectable marker produces a fluorescent, luminescent, or colorimetric-signal.

17. The cell of claim 16, wherein the detectable marker is selected from the group consisting of: green fluorescent protein, an enhanced green fluorescent protein, blue fluorescent protein, yellow fluorescent protein, cyan fluorescent protein.

18. The cell of claim 16, wherein the detectable marker comprises a region that targets the marker for intracellular proteolysis.

19. The cell of claim 18, wherein the region is a PEST domain.

20. The cell of claim 18, wherein the detectable marker is a fusion protein having the region that targets the marker for increased intracellular proteolysis located at its N-terminus or C-terminus.

21. The cell of claim 16, wherein the detectable marker has a half-life of approximately 2 hours or less.

22. The cell of claim 16, wherein the detectable marker has a half-life of approximately 1 hour or less.

23. The cell of claim 16, wherein the detectable marker is enhanced GFP having increased expression level, increased fluorescence intensity, decreased half-life, or a combination of any of the foregoing relative to wild type GFP.

24. The cell of claim 23, wherein the enhanced GFP has a decreased half-life relative to wild type GFP.

25. The cell of claim 1, wherein the nucleic acid that encodes a selectable marker is integrated into the genome of the cell.

26. The cell of claim 1, wherein expression of the selectable marker confers a growth advantage under a first set of selective conditions and confers a growth disadvantage under a second set of selective conditions.

27. The cell of claim 1, wherein the selectable marker is selected from the group consisting of: proteins that catalyze a step in a nucleotide synthesis or salvage pathway, DNA repair proteins, DNA synthesis proteins, RNA synthesis proteins, proteins that expel a cytotoxic compound out of a cell, and proteins that alter the permeability of a cell to a cytotoxic compound.

28. The cell of claim 1, wherein the selectable marker is selected from the group consisting of: HPRT, TK, DHFR, and MDR family members.

29. The cell of claim 1, wherein the selectable marker is HPRT.

30. The cell of claim 1, wherein the selectable marker confers resistance to an antibiotic.

31. The cell of claim 1, wherein the selectable marker confers resistance to a chemotherapeutic agent.

32. The cell of claim 31, wherein the chemotherapeutic agent is selected from the group consisting of: methotrexate, vinblastine, and anthracyclines.

33. The cell of claim 1, further comprising:
(i) a nucleic acid that encodes a second marker, wherein the marker is a selectable or detectable marker;
(ii) one or more nucleic acid templates for transcription of an RNAi-inducing agent, wherein the RNAi-inducing agent reduces expression of the second marker, and wherein the templates for transcription of RNAi-inducing agents that reduce expression of the markers are integrated into the genome of the cell as a single unit.

34. A cell comprising:
(i) a nucleic acid that encodes a detectable marker, wherein the detectable marker has a half-life of approximately 2 hours or less; and
(ii) a template for transcription of an RNAi-inducing agent that reduces expression of the detectable marker integrated into the genome of the cell, wherein the RNAi-inducing agent reduces expression of the marker and is not naturally found in the cell.

35. The cell of claim 34, wherein the detectable marker has a half-life of approximately 1 hour or less.

36. The cell of claim 34, wherein the marker is enhanced GFP having increased expression level, increased fluorescence intensity, decreased half-life, or a combination of any of the foregoing relative to wild type GFP.

37. The cell of claim 36, wherein the enhanced GFP has a decreased half-life relative to wild type GFP.

38. A mammalian cell comprising:
a nucleic acid that is integrated into the genome of the cell and provides a template for transcription of an mRNA transcript that encodes a detectable or selectable marker, wherein the mRNA transcript comprises one or more binding sites for an endogenous miRNA or an miRNA-like RNA; and
an endogenous miRNA or miRNA-like RNA that is expressed by the cell and represses translation of the mRNA that encodes a detectable or selectable marker.

39. The mammalian cell of claim 38, wherein the cell expresses an siRNA or shRNA that is targeted to the mRNA transcript.

40. A cell line comprising a plurality of cells as set forth in claim 1, 2, 5, 10, 15, 26, 33, 34, or 39, wherein the cells are descended from a single cell.

41. A collection of cell lines wherein cells of each cell line comprise:
(i) a nucleic acid that encodes a marker, wherein the nucleic acid in cells of each cell line encodes the same marker; and
(ii) a template for transcription of an RNAi-inducing agent that reduces expression of the marker, wherein the RNAi-inducing agent reduces expression of the marker to different extents in each of the cell lines.

42. The collection of cell lines of claim 41, wherein each of the cell lines is derived from a single cell.

43. The collection of cell lines of claim 41, wherein the RNAi-inducing agent in each of the cell lines is the same.

44. The collection of cell lines of claim 41, wherein the cell lines comprise cells of the same cell type.

45. The collection of cell lines of claim 41, wherein the cell lines are human cell lines.

46. The collection of cell lines of claim 41, wherein the cell lines are non-human cell lines.

47. The collection of cell lines of claim 41, wherein the marker is a detectable marker.

48. The collection of cell lines of claim 41, wherein the detectable marker has a half-life of approximately an hour or less.

49. The collection of cell lines of claim 41, wherein the marker is enhanced GFP having increased expression level, increased fluorescence intensity, decreased half-life, or a combination of any of the foregoing relative to wild type GFP.

50. The collection of cell lines of claim 49, wherein the enhanced GFP has a decreased half-life relative to wild type GFP.

51. The collection of cell lines of claim 41, further comprising a cell line that comprises a nucleic acid that encodes the marker but does not comprise a template for transcription of an RNAi-inducing agent that reduces expression of the marker.

52. A kit comprising the collection of cell lines of claim 41 and one or more items selected from the group consisting of:
(i) an RNAi-inducing agent that targets an mRNA that encodes the marker;
(ii) an RNAi-inducing agent that does not target an mRNA that encodes the marker;
(iii) a compound that inhibits RNAi;
(iv) a compound that activates RNAi;
(v) a genetic element that inhibits RNAi;
(vi) a genetic element that activates RNAi;
(vii) an RNAi-inducing agent that targets an mRNA that encodes Dicer;
(viii) a cell line comprising a plurality of the mammalian cell of claim 1;
(ix) one or more compounds for addition to tissue culture medium to impose a selective condition on the mammalian cell line of (viii);
(x) a cell line that comprises a nucleic acid that encodes the same marker as the collection of cell lines but does not comprise a template for transcription of an RNAi-inducing agent that reduces expression of the marker;
(xi) a vector comprising a U6, H1, or tRNA promoter and a site downstream of the promoter for insertion of a template for transcription of an RNAi-inducing agent;
(xii) a transfection reagent; and
(xiii) instructions for use.

53. The kit of claim 52, wherein the marker of the collection of cell lines is a detectable marker.

54. A kit comprising a cell line comprising a plurality of cells as set forth in claim 1 and one or more items selected from the group consisting of:
(i) an RNAi-inducing agent that targets an mRNA that encodes the selectable marker;
(ii) an RNAi-inducing agent that does not target an mRNA that encodes the selectable marker;
(iii) a cell line that comprises a nucleic acid that encodes the same marker as the cell line comprising a plurality of cells as set forth in claim 1 but does not comprise a template for transcription of an RNAi-inducing agent that reduces expression of the marker;
(iv) one or more of the cell lines of the collection of cell lines of claim 41;
(v) an RNAi-inducing agent that targets an mRNA that encodes the marker of the cell line of (iv);
(vi) a compound that inhibits RNAi;
(vii) a compound that activates RNAi;
(viii) a genetic element that inhibits RNAi;
(ix) a genetic element that activates RNAi;
(x) an RNAi-inducing agent that targets an mRNA that encodes Dicer;
(xi) one or more compounds for addition to tissue culture medium to impose a selective condition on the cell line comprising a plurality of the cell of claim 1;
(xii) a cell line that comprises a nucleic acid that encodes the same marker as the collection of cell lines but does not comprise a template for transcription of an RNAi-inducing agent that reduces expression of the marker;
(xiii) a vector comprising a U6, H1, or tRNA promoter and a site downstream of the promoter for insertion of a template for transcription of an RNAi-inducing agent; and
(xiv) instructions for use.

55. The kit of claim 54, wherein the marker of the collection of cell lines is a detectable marker.

56. A cell comprising:
(i) a nucleic acid that encodes a marker, wherein expression of the marker increases or decreases sensitivity of the cell to a compound or environmental condition so that growth of the cell is inhibited or enhanced, respectively, in the presence of the compound or environmental condition relative to growth in its absence; and
(ii) one or more templates for transcription of an RNAi-inducing agent that reduces expression of the marker so that growth of the cell in the presence of the compound or environmental condition is increased or decreased relative to growth in its absence.

57. The cell of claim 56, wherein expression of the marker increases sensitivity of the cell to a compound or environmental condition so that growth of the cell is inhibited in the presence of the compound or environmental condition relative to growth in its absence, and wherein the RNAi-inducing agent reduces expression of the marker so that growth of the cell in the presence of the compound or environmental condition is increased relative to growth in its absence.

58. The cell of claim 56, wherein expression of the marker decreases sensitivity of the cell to a compound or environmental condition so that growth of the cell is enhanced in the presence of the compound or environmental condition relative to growth in its absence, and wherein the RNAi-inducing agent reduces expression of the marker so that growth of the cell in the presence of the compound or environmental condition is decreased relative to growth in its absence.

59. The cell of claim 56, wherein the marker is selected from the group consisting of: HPRT, TK, DHFR, and MDR family members.

60. A nucleic acid comprising:
(i) a template for transcription of a first RNAi-inducing agent targeted to an mRNA that encodes a first marker, wherein the template is operably linked to a promoter active in a mammalian cell; and
(ii) a template for transcription of a second RNAi-inducing agent targeted to an mRNA that encodes a second marker, wherein the template is operably linked to a promoter active in a mammalian cell.

61. The nucleic acid of claim 60, wherein transcription from the promoters is driven by RNA polymerase I or RNA polymerase III.

62. The nucleic acid of claim 61, wherein the promoter is selected from the group consisting of: the U6 promoter, the H1 promoter, and tRNA promoters.

63. The nucleic acid of claim 60, wherein transcription from at least one of the promoters is driven by RNA polymerase II.

64. The nucleic acid of claim 60, wherein one or both of the RNAi-inducing agents are shRNAs.

65. The nucleic acid of claim 60, wherein one or both of the RNAi-inducing agents are siRNAs or precursors thereof.

66. The nucleic acid of claim 60, wherein one or both of the RNAi-inducing agents are precursors of miRNAs.

67. The nucleic acid of claim 60, wherein one or both of the markers is a selectable marker.

68. The nucleic acid of claim 60, wherein one or both of the markers is a detectable marker.

69. The nucleic acid of claim 60, wherein one of the markers is a detectable marker and the other marker is a selectable marker.

70. A vector comprising the nucleic acid of claim 60.

71. A mammalian cell comprising the nucleic acid of claim 60.

72. A cell line comprising a plurality of cells as set forth in claim 71.

73. A nucleic acid comprising (i) a template for transcription of a first RNAi-inducing agent targeted to a selectable or detectable marker and operably linked to a first promoter and (ii) a second promoter and a site for insertion of a template for transcription of an RNAi-inducing agent located downstream of the promoter, so that the template will be operably linked to the promoter once inserted.

74. The nucleic acid of claim 73, further comprising a region that encodes the selectable or detectable marker.

75. The nucleic acid of claim 73, wherein the marker is a selectable marker selected from the group consisting of HPRT, TK, or an MDR family member.

76. A vector comprising the nucleic acid of claim 73.

77. A mammalian cell comprising the nucleic acid of claim 73.

78. A cell line comprising a plurality of cells as set forth in claim 77.

79. A method of identifying a cell in which a gene of interest is silenced by RNAi comprising steps of:
(i) introducing the nucleic acid of claim 73 into a population of cells, wherein the nucleic acid further comprises a template for transcription of an RNAi-inducing agent targeted to the gene of interest; and
(ii) identifying a cell in which RNAi is active by selecting or detecting cells that do not express the selectable or detectable marker, thereby identifying a cell in which the gene of interest is silenced by RNAi.

80. The method of claim 79, wherein the marker is a selectable marker and the step of identifying comprises exposing the cells to selective conditions that select against cells that express the selectable marker.

81. The method of claim 79, wherein the marker is an endogenous gene.

82. The method of claim 79, wherein the marker is selected from the group consisting of HPRT, TK, or an MDR family member.

83. The method of claim 79, wherein the step of identifying comprises exposing the cells to a compound that is processed by the selectable marker to yield a toxic compound.

84. The method of claim 79, wherein the marker is a detectable marker and the step of identifying comprises detecting a cell that does not express the marker.

85. A method of identifying a gene involved in an RNAi pathway comprising steps of:
(a) providing a population of mammalian cells members of which comprise a nucleic acid that encodes a detectable or selectable marker and further comprise one or more templates for transcription of an RNAi-inducing agent that reduces expression of the detectable or selectable marker;
(b) mutagenizing the population of cells; and
(c) identifying cells that display decreased or increased expression of the detectable or selectable marker relative to the starting population, thereby identifying cells that have a mutation in a gene involved in an RNAi pathway.

86. The method of claim 85, wherein the identifying step comprises identifying cells that display decreased expression of the marker relative to the starting population, thereby identifying cells that have a gain of function mutation in a gene involved in an RNAi pathway.

87. The method of claim 85, wherein the identifying step comprises identifying cells that display increased expression of the marker relative to the starting population, thereby identifying cells that have a loss of function mutation in a gene involved in an RNAi pathway.

88. The method of claim 85, wherein the marker is a selectable marker.

89. The method of claim 88, wherein the marker is selected from the group consisting of: HPRT, TK, DHFR, and MDR family proteins.

90. The method of claim 85, wherein the marker is a detectable marker.

91. The method of claim 90, wherein the detectable marker comprises a domain that increases intracellular proteolysis of the marker.

92. The method of claim 85, wherein the cell comprises a nucleic acid that encodes a detectable marker and a nucleic acid that encodes a selectable marker.

93. The method of claim 85, wherein the step of mutagenizing is performed by exposing the cells to a chemical mutagen or to radiation.

94. The method of claim 85, wherein the step of mutagenizing comprises insertional mutagenesis.

95. The method of claim 94, wherein insertional mutagenesis is performed by infecting the cells with a retrovirus.

96. The method of claim 95, further comprising the step of recovering the virus.

97. The method of claim 85, further comprising the step of:
performing a secondary screen or selection wherein the secondary screen or selection assesses the ability of an RNAi-inducing agent targeted to a second marker to inhibit expression of the second marker in the cell.

98. The method of claim 97, wherein the second marker is a detectable marker.

99. The method of claim 97, wherein the second marker is a selectable marker.

100. The method of claim 85, further comprising the step of:
cloning the gene having the mutation.

101. A method of identifying a gene involved in a miRNA translational repression pathway comprising steps of:
(a) providing a population of mammalian cells members of which comprise (i) a nucleic acid that is integrated into the genome of the cell and provides a template for transcription of an mRNA transcript that encodes a detectable or selectable marker, wherein the mRNA transcript comprises one or more binding sites for an endogenous miRNA or an miRNA-like RNA; and (ii) an endogenous miRNA or miRNA-like RNA that is expressed by the cell and represses translation of the mRNA that encodes a detectable or selectable marker;
(b) mutagenizing the population of cells; and
(c) identifying cells that display decreased or increased expression of the detectable or selectable marker relative to the starting population and do not display an alteration in mRNA transcript level sufficient to account for the increased or decreased expression of the marker, thereby identifying cells that have a mutation in a gene involved in an miRNA translational repression pathway.

102. The method of claim 101, further comprising the step of:
cloning the gene.

103. A method for identifying cells containing a genetic element that inhibits or activates an RNAi pathway comprising steps of:
(a) providing a first population of mammalian cells members of which comprise a nucleic acid that encodes a first detectable or selectable marker and express an RNAi-inducing agent that reduces expression of the detectable or selectable marker;
(b) introducing a library into the population of cells, wherein the library comprises a plurality of genetic elements;
(c) identifying cells that display increased or decreased expression of the detectable or selectable marker relative to the starting population, thereby identifying cells that contain a genetic element that inhibits or activates an RNAi pathway, respectively.

104. The method of claim 103, wherein the identifying step comprises identifying cells that display increased expression of the detectable marker relative to the starting population, thereby identifying cells that contain a genetic element that inhibits an RNAi pathway.

105. The method of claim 103, wherein the identifying step comprises identifying cells that display decreased expression of the detectable marker relative to the starting population, thereby identifying cells that contain a genetic element that activates an RNAi pathway.

106. The method of claim 103, wherein the genetic element is a genetic suppressor element.

107. The method of claim 103, wherein the marker is a selectable marker.

108. The method of claim 103, wherein the marker is a detectable marker.

109. The method of claim 103, further comprising the step of identifying the genetic element.

110. The method of claim 109, wherein the step of identifying the genetic element comprises PCR amplification.

111. The method of claim 109, wherein the step of identifying the genetic element comprises sequencing.

112. The method of claim 103 or 109, further comprising the step of identifying the gene suppressed by the genetic element, thereby identifying a gene involved in an RNAi pathway.

113. The method of claim 103, wherein the step of introducing the library comprises retroviral infection.

114. The method of claim 103, wherein the step of introducing the library comprises DNA transfection.

115. The method of claim 103, wherein the library is a cDNA library.

116. The method of claim 115, wherein the cDNAs are size-selected.

117. The method of claim 115, wherein the cDNAs are normalized.

118. The method of claim 115, wherein some or all of the cDNAs encode protein fragments.

119. The method of claim 115, wherein some or all of the cDNAs are operably linked in reverse orientiation to a promoter so that transcription results in synthesis of antisense RNA molecules.

120. The method of claim 103, wherein the templates for transcription are inserted into a vector comprising an episomal element that replicates within mammalian cells.

121. The method of claim 103, wherein the templates for transcription are inserted into a retroviral vector.

122. The method of claim 103, further comprising the step of:
performing a secondary screen or selection.

123. The method of claim 122, wherein the secondary screen or selection assesses the ability of an RNAi-inducing agent targeted to a second marker to inhibit expression of the second marker in the cell.

124. The method of claim 122, wherein the second marker is a detectable marker.

125. The method of claim 122, wherein the second marker is a selectable marker.

126. The method of claim 122, wherein the secondary screen or selection comprises:
(i) introducing the genetic element into a second population of mammalian cells members of which comprise a nucleic acid that encodes a second detectable or selectable marker and express an RNAi-inducing agent that reduces expression of the second detectable or selectable marker; and
(ii) assessing expression of the second detectable or selectable marker.

127. The method of claim 126, wherein the second marker is a detectable marker and the step of assessing expression of the marker comprises detecting the marker.

128. The method of claim 126, wherein the second marker is a selectable marker and the step of assessing expression of the marker comprises detecting cell growth under selective conditions.

129. A genetic element identified according to the method of claim 109.

130. A protein fragment encoded by a genetic element identified according to the method of claim 109.

131. An antisense molecule complementary to a genetic element identified according to the method of claim 109.

132. A method of inhibiting or activating RNAi in a cell comprising:
contacting the cell with a genetic element identified according to the method of claim 109.

133. A method for identifying cells containing a genetic element that inhibits or activates a miRNA translational repression pathway comprising steps of:
(a) providing a first population of mammalian cells members of which comprise (i) a nucleic acid that is integrated into the genome of the cell and provides a template for transcription of an mRNA transcript that encodes a detectable or selectable marker, wherein the mRNA transcript comprises one or more binding sites for an endogenous miRNA or an miRNA-like RNA; and (ii) an endogenous miRNA or miRNA-like RNA that is expressed by the cell and represses translation of the mRNA that encodes a detectable or selectable marker;
(b) introducing a library into the population of cells, wherein the library comprises a plurality of genetic elements;
(c) identifying cells that display increased or decreased expression of the detectable or selectable marker relative to the starting population and do not display an alteration in mRNA transcript level sufficient to account for the increased or decreased expression of the marker, thereby identifying cells that contain a genetic element that inhibits or activates an miRNA translational repression pathway, respectively.

134. The method of claim 133, further comprising the step of identifying the genetic suppressor element.

135. A method for identifying a compound that inhibits or activates RNA interference comprising steps of:
(a) providing a population of mammalian cells members of which comprise a nucleic acid that encodes a detectable or selectable marker and express an RNAi-inducing agent that reduces expression of the detectable or selectable marker by RNA interference;
(b) contacting the cells with a compound; and
(c) identifying the compound as an inhibitor of RNAi if cells exhibit enhanced expression of the detectable or selectable marker after being contacted with the compound relative to cells not contacted with the compound or identifying the compound as an activator of RNAi if cells exhibit reduced expression of the detectable or selectable marker after being contacted with the compound relative to cells not contacted with the compound.

136. The method of claim 135, wherein the marker is a detectable marker.

137. The method of claim 135, wherein the marker is a selectable marker, and wherein enhanced expression of the selectable marker confers a growth advantage on cells expressing the marker under a selective condition, and wherein the step of identifying comprises exposing the contacted cells to the selective condition and isolating cells that survive or proliferate under the selective condition.

138. The method of claim 135, wherein the compound is a member of a compound library, and wherein the method comprises contacting a plurality of portions of the population with individual members of the compound libary and identifying one or more compounds as an inhibitor or activator of RNAi.

139. The method of claim 138, wherein the library is an annotated compound library.

140. The method of claim 138, wherein the library is a library of small molecules.

141. The method of claim 138, wherein the library is a natural product library.

142. The method of claim 138, wherein the library is a combinatorial library.

143. The method of claim 135, further comprising the step of: performing a secondary screen or selection to retest the compound.

144. The method of claim 143, wherein the secondary screen or selection comprises:
(i) contacting a second population of mammalian cells members of which comprise a nucleic acid that encodes a second detectable or selectable marker and express an RNAi-inducing agent that reduces expression of the second detectable or selectable marker with the compound; and
(ii) assessing expression of the second detectable or selectable marker.

145. The method of claim 143, wherein the second marker is a detectable marker and the step of assessing expression of the marker comprises detecting the marker.

146. The method of claim 143, wherein the second marker is a selectable marker and the step of assessing expression of the marker comprises detecting cell growth under selective conditions.

147. The method of claim 135, further comprising the steps of:
(i) modifying the compound; and
(ii) testing the ability of the modified compound to activate or inhibit RNAi.

148. A compound identified according to the method of claim 135.

149. The compound of claim 148, further comprising an RNAi-inducing agent.

150. A method of inhibiting or activating RNAi in a cell comprising:
contacting the cell with the compound of claim 148.

151. A method of treating a subject comprising:
administering a therapeutic RNAi-inducing entity to the subject; and
administering a compound that activates an RNAi pathway to the subject.

152. The method of claim 152, wherein the compound is identified according to the method of claim 135.

153. A method for identifying a compound that inhibits or activates an miRNA translational repression pathway comprising steps of:
(a) providing a population of mammalian cells members of which comprise (i) a nucleic acid that is integrated into the genome of the cell and provides a template for transcription of an mRNA transcript that encodes a detectable or selectable marker, wherein the mRNA transcript comprises one or more binding sites for an endogenous miRNA or an miRNA-like RNA; and (ii) an endogenous miRNA or miRNA-like RNA that is expressed by the cell and represses translation of the mRNA that encodes a detectable or selectable marker;
(b) contacting the cells with a compound; and
(c) identifying the compound as an inhibitor of a miRNA translational repression pathway if cells exhibit enhanced expression of the detectable or selectable marker after being contacted with the compound relative to cells not contacted with the compound and do not display enhanced mRNA transcript levels sufficient to account for the enhanced expression of the marker, or identifying the compound as an activator of an miRNA translational repression pathway if cells exhibit reduced expression of the detectable or selectable marker after being contacted with the compound relative to cells not contacted with the compound and do not display decreased mRNA transcript levels sufficient to account for the reduced expression of the marker.

154. A computer-readable medium containing computer-readable information indicating that a gene, mutation, genetic element, or compound affects an RNAi pathway, wherein the gene, mutation, genetic element, or compound was identified as affecting an RNAi pathway by the method of claim 85, 101, 103, 133, 135, or 153.


SEQUENCE LISTING (29 sequences)

SEQ ID NO: 1 - DNA, 53 bp, Artificial
  Aequoria victoria (green fluorescent protein)
ggctacgtccaggagcgcaccctcgagggtgcgctcctggacgtagccttttt

SEQ ID NO: 2 - DNA, 56 bp, Artificial
  Aequoria victoria (green fluorescent protein)
aattcaaaaaggctacgtccagggcgaccctcgagggtgcgctcctggacggagcc

SEQ ID NO: 3 - DNA, 65 bp, Artificial
  Aequoria victoria (green fluorescent protein)
aattccctcaaccagccactgctggattcaagagatccagcagtggctggttgatttttctcgag

SEQ ID NO: 4 - DNA, 65 bp, Artificial
  Aequoria victoria (green fluorescent protein)
gatcctcgagaaaaatcaaccagccactgctggatctcttgaatccagcagtggctggttgaggg

SEQ ID NO: 5 - DNA, 21 bp, Artificial
  Aequoria victoria (green fluorescent protein)
gugucauuagugaaacuggaa

SEQ ID NO: 6 - DNA, 21 bp, Artificial
  Aequoria victoria (green fluorescent protein)
ccaguuucacuaaugacacaa

SEQ ID NO: 7 - DNA, 55 bp, Artificial
  Aequoria victoria (green fluorescent protein)
cttacgctgagtacttcgaaactcgagtttcgaagtactcagcgtaagttttttg

SEQ ID NO: 8 - DNA, 50 bp, Artificial
  Mouse or human
ggugucauuagugaaacuggcucgagccaguuucacuaaugacaccuuuu

SEQ ID NO: 9 - DNA, 53 bp, Artificial
  Mouse or human
gggcuacguccaggagcgcauucaagagaugcgcuccuggacguagccuuuuu

SEQ ID NO: 10 - DNA, 45 bp, Artificial
  Mouse or human
gggccctctagactcgagcggccgccactgtgctggatatctgca

SEQ ID NO: 11 - DNA, 56 bp, Artificial
  Mouse or human
gaattccaccacactggactagtggatccgagctcggtaccaagcttaagtttaaa

SEQ ID NO: 12 - DNA, 53 bp, Artificial
  Mouse or human
ggctacgtccaggagcgcattcaagagatgcgctcctggacgtagcctttttg

SEQ ID NO: 13 - DNA, 57 bp, Artificial
  Mouse or human
aattcaaaaaggctacgtccaggagcgcatctcttgaatacgctcctggacgtagcc

SEQ ID NO: 14 - DNA, 53 bp, Artificial; identical to SEQ ID NO: 12
  Mouse or human

SEQ ID NO: 15 - DNA, 57 bp, Artificial
  Mouse or human
ccgatgcaggtcctcgcgtaagttctctacgcgaggacctgcatcggaaaaacttaa

SEQ ID NO: 16 - DNA, 53 bp, Artificial; identical to SEQ ID NO: 12
  Mouse or human

SEQ ID NO: 17 - DNA, 57 bp, Artificial; identical to SEQ ID NO: 15
  Mouse or human

SEQ ID NO: 18 - DNA, 53 bp, Artificial; identical to SEQ ID NO: 12
  Mouse or human

SEQ ID NO: 19 - DNA, 57 bp, Artificial
  Mouse or human
aattcaaaaaggctacgtccaggagcgcatctcttgaatgcgctcctggacgtagcc

SEQ ID NO: 20 - DNA, 59 bp, Artificial
  Mouse or human
gggctacgtccaggagcgcattcaagagatgcgctcctggacgtagcctttttgaattc

SEQ ID NO: 21 - DNA, 59 bp, Artificial
  Mouse or human
gaattcaaaaaggctacgtccaggagcgcatctcttgaatgcgctcctggacgtagccc

SEQ ID NO: 22 - DNA, 53 bp, Artificial; identical to SEQ ID NO: 9
  Mouse or human

SEQ ID NO: 23 - DNA, 52 bp, Artificial
  Mouse or human
ggugucauuagugaaacugguucaagagaccaguuucacuaaugacacuuuu

SEQ ID NO: 24 - DNA, 69 bp, Artificial
  Mouse or human
actccttctctaggcgccggaattggccattaaggcctgcaggatccggccgcctcggccgatccggct

SEQ ID NO: 25 - DNA, 69 bp, Artificial
  Mouse or human
tgaggaagagatccgcggccttaaccggtaattccggacgtcctaggccggcggagccggctaggccga

SEQ ID NO: 26 - DNA, 72 bp, Artificial
  Mouse or human
ugucggguagcuuaucagacugauguugacuguugaaucucauggcaacaccagucgaugggcugucugaca

SEQ ID NO: 27 - DNA, 71 bp, Artificial
  Mouse or human
gcgacuguaaacauccucgacuggaagcugugaagccacagaugggcuuucagucggauguuugcagcugc

SEQ ID NO: 28 - DNA, 21 bp, Artificial
  Mouse or human
ucaacaucagaagauaagcua

SEQ ID NO: 29 - DNA, 22 bp, Artificial
  Mouse or human
aguuguagucagacuauucgau